(19) United States
(12) Patent Application Publication
(10) Pub. No.: US 2026/0201068 A1
(43) Pub. Date: Jul. 16, 2026

(54) CHIMERIC ANTIGEN RECEPTOR

(71) Applicants: Université de Genève, Genève (CH); Les Hôpitaux universitaires de Genève, Genève (CH)

(72) Inventors: Denis MIGLIORINI, Genève (CH); Valérie DUTOIT, Genève (CH); Pierre-Yves DIETRICH, Genève (CH); Darel MARTINEZ BEDOYA, Genève (CH)

(73) Assignees: Université de Genève, Genève (CH); Les Hôpitaux universitaires de Genève, Genève (CH)

(21) Appl. No.: 19/136,826

(22) PCT Filed: Dec. 8, 2023
(86) PCT No.: PCT/EP2023/084961

§ 371 Date: Jun. 6, 2025

(30) Foreign Application Priority Data
Dec. 8, 2022 (EP) 22212246.7

(51) Int. Cl. C07K 16/40; A61K 40/11; A61K 40/31; A61K 40/42; A61P 35/00; C07K 16/18; C07K 16/30; C12N 5/0783

(52) U.S. Cl. CPC C07K 16/40; A61K 40/11; A61K 40/31; A61K 40/42; A61K 40/4252; A61K 40/4261; A61P 35/00; C07K 16/18; C07K 16/3053; C12N 5/0636; C12Y 301/03048; C07K 2317/35; C07K 2317/53; C07K 2317/569; C07K 2317/622; C07K 2319/03; C12N 2510/00

(57) ABSTRACT

The disclosure relates to a chimeric antigen receptors (CARs) specific for one or more glioma-associated antigens and an immune effector cell or a population of immune effector cells expressing one or more CARs specific for one or more glioma-associated antigens. The disclosure also relates to a method of making the immune cell or population, a method of treating cancer using the immune cell or population.

### FIELD OF THE INVENTION

The disclosure relates to immune effector cells expressing one or more chimeric antigen receptors and their use in the treatment of glioma.

### BACKGROUND OF INVENTION

Immunotherapeutic approaches that induce tumour-specific immune responses are being considered across multiple malignancies. For instance, therapeutic T cells may be engineered to direct their cytotoxic effects towards a particular antigen of interest. In this way, T cells responsible for killing tumour cells may be engineered to be specific for a tumour antigen. The specificity of a T cell may be directed by endogenous or recombinant T cell receptors or by chimeric antigen receptors (CARs).

CARs are synthetic receptors comprising an extracellular domain, often derived from an antibody single-chain variable fragment (scFv), and intracellular signalling and costimulatory domains derived from T cells. Genetic insertion of CARs into immune cells allows redirecting them to a desired antigen. Anti-CD19 CAR T cells led to a paradigm change in cancer therapy, based on their response rates in adult patients with recurrent/refractory diffuse large B cell lymphoma (DLBCL) or paediatric refractory B cell acute lymphoblastic leukaemia (B-ALL). Two CAR T cell products specific for the B-cell marker CD19, Kymriah (Novartis) and Yescarta (Kite Pharma), became the first therapeutic products registered by the FDA comprising a genetic engineering element for the treatment of B-ALL and DLBCL.

Glioblastoma (GBM, grade IV astrocytoma) is the most frequent and aggressive primary malignant tumour originating in the brain. Despite treatments involving a combination of surgery, chemotherapy and radiotherapy, the overall survival of GBM patients is 14.6 to 16 months post-treatment. Several phase I/II studies using multipeptide vaccines or neoadjuvant immune checkpoint blockers have been tested, with limited success thus far. At present, CAR T cell approaches have been investigated for GBM targeting EGFRvIII, IL-13Ra2 and Her2 (Brown et al., New England Journal of Medicine 375.26 (2016): 2561-2569; Ahmed et al., JAMA oncology 3.8 (2017): 1094-1101; O'Rourke et al., Science translational medicine 9.399 (2017): eaaa0984).

It is an object of the invention to develop further glioma antigen-specific CARs and improved immune effector cells expressing CARs, useful for treating glioma.

### SUMMARY OF INVENTION

The invention provides an immune effector cell or a population of immune effector cells expressing one or more chimeric antigen receptors (CARs) specific for two or more glioma-associated antigens, wherein one or more of the glioma-associated antigens are selected from PTPRZ1, BCAN, CSPG4 and TNC.

The invention further provides an immune effector cell or a population of immune effector cells expressing a chimeric antigen receptor (CAR) specific for PTPRZ1. The invention also provides an immune effector cell or a population of immune effector cells expressing a chimeric antigen receptor (CAR) specific for BCAN.

The invention also provides a method of making an immune effector cell or a population of immune effector cells of the invention, comprising transforming said cell or said population of cells with one or more nucleic acids encoding one or more CARs specific for one or more glioma-associated antigens.

The invention further provides a method of treating cancer in a subject, comprising administering to the subject an effective amount of an immune effector cell or a population of immune effector cells of the invention. The invention also provides the immune effector cell or the population of immune effector cells of the invention for use in a method of treating cancer in a subject.

The invention additionally provides a CAR specific for PTPRZ1. The invention also provides a CAR specific for BCAN. The invention also provides a CAR specific for a glioma-associated antigen selected from PTPRZ1, BCAN, CSPG4 and TNC, comprising a polypeptide having (a) the heavy chain complementarity determining regions (HCDR1, HCDR2 and HCDR3) and the light chain complementarity determining regions (LCDR1, LCDR2 and LCDR3) from an amino acid sequence selected from any one of SEQ ID NOs: 21-40, or (b) the complementary determining regions (CDR1, CDR2 and CDR3) from an amino acid sequence selected from any one of SEQ ID NOs: 83-93.

The invention also provides a multivalent CAR comprising (a) an extracellular domain specific for two or more glioma-associated antigens, wherein one or more of the glioma-associated antigens are selected from PTPRZ1, BCAN, CSPG4 and/or TNC; and (b) and an intracellular signalling domain.

The invention also provides a nucleic acid encoding a CAR or multivalent CAR of the invention. A vector comprising one or more of said nucleic acids is also provided by the invention.

The invention also provides an antigen binding molecule specific for one or more glioma-associated antigens, which comprises a polypeptide having the complementary determining regions (CDR1, CDR2 and CDR3) from an amino acid sequence selected from any one of SEQ ID NOs: 83-93.

### BRIEF DESCRIPTION OF FIGURES

FIG. 1: Generation of a PTPRZ1-overexpressing GBM cell line. A) GBM cell line Ge518 was transfected with a 3rd generation lentivirus to introduce the extracellular domains 1 and 2 of human PTPRZ1. Antigen expression in Ge518 wt (light blue) and Ge518 PTPRZ1-KI (dark blue) was measured by flow cytometry. B) Ge518_PTPRZ1-KI staining by flow cytometry using six different anti-PTPRZ1 scFv.

FIG. 2: Killing activity of anti-PTPRZ1_BBz RNA CAR T cells against Ge518_PTPRZ1-KI cells. A) CAR expression of six different anti-PTPRZ1 BBz and control anti-IL13Ra2_BBz RNA CAR T cells. NTD: non-transduced T cells. B) Killing activity, measured by flow cytometry as percentage of specific lysis, of the anti-PTPRZ1 BBz RNA CAR T cells against Ge518_PTPRZ1-KI tumor cells. E:T ratio=effector to target ratio.

FIG. 3: Comparison of the killing capacity of BBz vs 28z variants of anti-PTPRZ1 RNA CAR T cells. A) Killing activity, measured by flow cytometry as percentage of specific lysis, of anti-PTPRZ1 RNA CAR T cells containing scFvs 473 and 476 and 28z or BBz, against Ge518_PTPRZ1-KI tumor cells. E:T ratio=effector to target ratio. B) Comparison of the killing activity of the anti-PTPRZ1 scFv 471 28z and BBz RNA CAR T cells against Ge518_PTPRZ1-KI tumor cells, at two different E:T ratios.

FIG. 4: Difference in killing activity of three different anti-PTPRZ1_28z RNA CAR T cells. Measure of the killing capacity of anti-PTPRZ1 28z RNA CAR T cells containing the 470, 471 and 476 scFvs against Ge518_PTPRZ1-KI tumor cells at three different E:T ratios. E:T ratio=effector to target ratio.

FIG. 5: Killing activity of anti-CSPG4_BBz RNA CAR T cells. A) Cell surface expression of six different anti-CSPG4 BBz CAR molecules and control anti-IL13Ra2_BBz RNA CAR. NTD: non-transduced T cells. B) Killing activity, measured by flow cytometry as percentage of specific lysis, of the anti-CSPG4_BBz RNA CAR T cells against A375 melanoma cells. E:T ratio=effector to target ratio. C) Killing activity of the anti-CSPG4_BBz RNA CAR T cells against Ge518 GBM cells. D) IFN-γ secretion in the supernatant of anti-CSPG4_BBz RNA CAR T cells incubated with Ge518 GBM cells.

FIG. 6: Generation of Ge518 variants KOs for IL13Ra2, Her2 and CSPG4 antigens. A) IL13Ra2, Her2 and CSPG4 antigen expression was eliminated in Ge518 cells using a CRISPR-Cas9 system. Antigen expression loss was verified by flow cytometry. B) Killing of the wt Ge518 cell line as well as the Ge518 KOs cell lines cognate RNA BBz CAR T cells was assessed by flow cytometry at an E:T ratio of 3:1.

FIG. 7: Cytotoxic activity of a combination of three anti-GBM RNA CAR T cells against a heterogeneous mix of tumor cells. A) Diagram of mechanism for better killing and less risk of antigen loss escape of a mix of different anti-GBM CAR T cells in comparison to mono-specific CAR T cells. B) Killing capacity of different mixes of anti-CSPG4_BBz, anti-Her2_BBz and anti-IL13Ra2_BBz CAR T cells against a heterogeneous mix of the three different Ge518 KO cells (IL13Ra2-KO, Her2-KO and CSPG4-KO) and the Ge518 wt cells. E:T ratio=effector: target ratio.

FIG. 8: Triple RNA CAR-T cell generation, and in vitro killing assay. (A) CAR expression on RNA-CAR T cells expressing anti-PTPRZ1 (471_28z), anti-CSPG4 (301_28z) and anti-BCAN (295_28z). (B) In vitro killing of Ge518, Ge518_BCANv2TM-KI and Ge518_PTPRZ1-KI cell lines by the Triple CAR or individual monovalent CAR-T cells. E:T ratio: 3:1 and 1:1. Mock EP cells were used as negative control. (C) In vitro killing of a MIX of Ge518, Ge518 BCANv2TM-KI and Ge518_PTPRZ1-KI cell lines (2:2:1 proportion) by the Triple CAR or individual monovalent CAR-T cells. ET ratio: 3:1 and 1:1. Mock EP cells were used as negative control. (D) In vitro inhibition of tumor growth was measured by Incucyte of Ge518, Ge518_BCANv2TM-KI and Ge518_PTPRZ1-KI cell lines by the Triple CAR or individual monovalent CAR-T cells. E:T ratio: 3:1. Mock EP cells were used as negative control. (E) In vitro inhibition of tumor growth was measured by Incucyte a MIX of Ge518, Ge518_BCANv2TM-KI and Ge518_PTPRZ1-KI cell lines (2:2:1 proportion) by the Triple CAR or individual monovalent CAR-T cells. E:T ratio: 3:1. Mock EP cells were used as negative control.

FIG. 9: 471_28z CAR-T cells did not show bystander killing capacity against non-tumoral human macrophages. (A) CD14+ monocytes were purified from human blood at day 0 and were differentiated to macrophages by a 6 days culture with M-CSF. At day 6, Ge518_PTPRZ1-KI cells (FarRed stained) were added to the macrophages culture, followed by the addition of anti-PTPRZ1 RNA 471_28z CAR-T cells or Mock EP control cells, at 3:1 E:T ratio. After 72h, cells were collected and cell dead of tumor and macrophages was evaluated by FACS. (B) CAR expression of anti-PTPRZ1 RNA 471 28z CAR-T cells. (C) Gating strategy to differentiate tumor Ge518_PTPRZ1-KI cells (CD14-FarRed+) and human macrophages (CD14+FarRed-) from wells treated with Mock EP (left) or 471 28z CAR-T cells (right). (D) In vitro evaluation of killing of Ge518_PTPRZ1-KI or human macrophages by anti-PTPRZ1 RNA 471_28z CAR-T cells.

FIG. 10: 471_28z CAR-T cells show a bystander killing capacity through soluble mediators. (A) At day 0, Ge518_PTPRZ1-KO cells were seeded at the bottom of a transwell plate, meanwhile, Ge518_PTPRZ1-KI cells were seeded on the top of the transwell. On Day 1, anti-PTPRZ1 RNA 471_28z CAR-T cells or Mock EP control cells were added on the top of the transwell at 5:1 E:T ratio. After 72h, cells on the bottom of the well were collected and cell dead was evaluated by FACS. (B) Evaluation of indirect, soluble mediator-dependent, killing of Ge518_PTPRZ1-KO cells (stained with Yellow dye) by anti-PTPRZ1 RNA 471 28z CAR-T cells. (C) Confirmation of absence of Ge518_PTPRZ1-KI cells (stained with FarRed) or T cells (CD3+) on the bottom part of wells.

FIG. 11: Correlation analysis between different antigens. (A) and (B) correlation between antigens. Lower diagonal: scatter plot with regression line. Middle diagonal: density plot. Upper diagonal: Pearson correlation coefficient with significance: ***<0.001, **<0.01, *<0.05. The test statistic is based on Pearson's product moment correlation coefficient. (A) Expression data from TCGA, bulk RNA-seq (primary GBM). (B) Expression data from CGGA (recurrent GBM). (C) Heatmap of expression data from TCGA, bulk RNA-seq (primary GBM), showing Pearson correlation coefficient between antigens.

FIG. 12: anti-PTPRZ1 VHH isolation, RNA CAR-T cell generation, and in vitro killing assay. (A) ELISA recognition of extracellular domains of PTPRZ1 by different anti-PTPRZ1 VHHs. (B) Recognition of Ge518_PTPRZ1-KI and Ge738 cell lines by anti-PTPRZ1 VHH conjugated to human IgG1 Fc domain (each VHH-Fc at 5 μg/mL). (C) CAR expression on RNA-CAR T cells expressing different anti-PTPRZ1 VHH, RB832 and RB833, with a short (_28z) or long (_IgG1H_28z) hinge followed by CD28 and CD35 domains. (D) In vitro killing of Ge518_PTPRZ1-KI and Ge738 cell lines by the four different VHH anti-PTPRZ1 RNA CAR-T cells as measured by flow cytometry. E:T ratio: 3:1. The anti-PTPRZ1 RNA CAR T cell based on scFv RRB471 was used as positive control meanwhile Mock EP cells were used as negative control. (E) In vitro inhibition of tumor growth was measured by Incucyte of Ge518_PTPRZ1-KI and Ge738 cell lines by the four different VHH anti-PTPRZ1 RNA CAR-T cells. ET ratio: 3:1. The anti-PTPRZ1 RNA CAR T cell based on scFv RRB471 was used as positive control meanwhile Mock EP cells were used as negative control.

FIG. 13: anti-CSPG4 VHH isolation, RNA CAR-T cell generation, and in vitro killing assay. (A) ELISA recognition of extracellular domains of CSPG4 by different anti-CSPG4 VHHs. (B) Recognition of A375 and Ge738 cell lines by anti-CSPG4 VHH conjugated to human IgG1 Fc domain (each VHH-Fc at 10 μg/mL). (C) CAR expression on RNA-CAR T cells expressing anti-CSPG4 VHH RB830 with a short (28z) or long (IgG1H_28z) hinge followed by CD28 and CD35 domains. (D) In vitro killing of A375, Ge518 and Ge738 cell lines by the two VHH anti-CSPG4 RB830 RNA CAR-T cells as measured by flow cytometry. E:T ratio: 3:1. The anti-CSPG4 RNA CAR T cell based on scFv HRB301 was used as positive control meanwhile Mock EP cells were used as negative control. (E) In vitro inhibition of tumor growth was measured by Incucyte of A375, Ge518 and Ge738 cell lines by the two VHH anti-CSPG4 RB830 RNA CAR-T cells. E:T ratio: 5:1. The anti-CSPG4 RNA CAR T cell based on scFv HRB301 was used as positive control meanwhile Mock EP cells were used as negative control.

FIG. 14: anti-Tenascin C VHH isolation, RNA CAR-T cell generation, and in vitro killing assay. (A) ELISA recognition of extracellular domains of Tenascin C by different anti-Tenascin C VHHs. (B) Recognition of Ge518 and Ge738 cell lines by anti-Tenascin C VHH conjugated to human IgG1 Fc domain (each VHH-Fc at 10 μg/mL), intracellular staining. (C) CAR expression on RNA-CAR T cells expressing different anti-Tenascin C VHH, RB835 and RB836, with a short (28z) or long (IgG1H_28z) hinge followed by CD28 and CD35 domains. (D) In vitro killing of Ge518 and Ge738 cell lines by the two different VHH anti-Tenascin C RNA CAR-T cells as measured by flow cytometry. E:T ratio: 3:1. The anti-Tenascin C RNA CAR T cell based on scFv R6N was used as positive control meanwhile Mock EP cells were used as negative control. (E) In vitro inhibition of tumor growth was measured by Incucyte of Ge518 and Ge738 cell lines by the two different VHH anti-Tenascin C RNA CAR-T cells. ET ratio: 5:1. The anti-Tenascin C RNA CAR T cell based on scFv R6N was used as positive control meanwhile Mock EP cells were used as negative control.

FIG. 15: anti-BCAN VHH isolation, RNA CAR-T cell generation, and in vitro killing assay. (A) ELISA recognition of BCAN by different anti-BCAN VHHs. (B) CAR expression on RNA-CAR T cells expressing different anti-BCAN VHH, RB826, RB827, RB828 and RB829, with a short (28z) or long (IgG1H_28z) hinge followed by CD28 and CD35 domains. (C) In vitro killing of Ge518_BCANv2TM-KI cell line by the eight different VHH anti-BCAN RNA CAR-T cells as measured by flow cytometry. E:T ratio: 3:1. The anti-BCAN RNA CAR T cell based on scFv HRB295 was used as positive control meanwhile Mock EP cells were used as negative control. (D) In vitro inhibition of tumor growth was measured by Incucyte of Ge518_BCANv2TM-KI cell line by the eight different VHH anti-BCAN RNA CAR-T cells. E:T ratio: 5:1. The anti-BCAN RNA CAR T cell based on scFv HRB295 was used as positive control meanwhile Mock EP cells were used as negative control.

FIG. 16: Bi-specific RNA CAR-T cells based in the combination of anti-PTPRZ1 VHH RB832 with a second CAR against CSPG4, BCAN or Tenascin C. The bar graph in the left column shows the gain in killing capacity of the bi-specific RNA CAR-T cells compared to the killing of monovalent anti-PTPRZ1 CAR against a cell line with low expression of PTPRZ1. The bar graph in the right column shows the gain in killing capacity of the bi-specific RNA CAR-T cells compared to the killing of the second monovalent CAR against a cell line with high expression of PTPRZ1. (A) Bi-specific CAR-T cells anti-PTPRZ1 (VHH RB832) plus anti-CSPG4 (VHH RB830). (B) Bi-specific CAR-T cells anti-PTPRZ1 (VHH RB832) plus anti-BCAN (scFv HRB295). (C) Bi-specific CAR-T cells anti-PTPRZ1 (VHH RB832) plus anti-Tenascin C (VHH RB835).

Description of Sequence Listing

SEQ ID NO: 1-anti-PTPRZ1 scFv “RRB469” nucleotide sequence
ATGGCCCAGGTGCAACTGGTGGAATCTGGGGGAGGCGTGGTTCAGCCTGGGAG
GTCCCTGCGGCTCTCCTGTGCAGCCTCAGGATTTACCTTCAGTAGCTACGCCAT
GCACTGGGTCCGCCAGGCTCCAGGCAAAGGGTTGGAATGGGTTGCAGTTATAT
CATATGATGGAAGTAATAAATACTACGCAGACTCCGTGAAGGGCCGATTCACC
ATTTCACGTGACAATTCCAAGAACACGCTTTATCTGCAAATGAACAGCTTGAG
AGCTGAAGATACGGCTGTGTATTACTGCGCGAGGGGTAGTGGATACAGCTATG
GTCCGGGTTATGATGCATTTGATATTTGGGGCCAGGGAACCCTTGTCACAGTCT
CAAGCGGTGGAGGCGGTTCAGGCGGAGGTGGCAGCGGCGGGGGCGGATCTAC
AAATTTTATGCTGACTCAGCCTCATTCTGTATCGGAGTCTCCAGGGAAGACAGT
AACCATCTCCTGCACACGCAGCAGTGGCAGCATCGCCAGCAACTATGTGCAGT
GGTACCAGCAGAGACCAGGCAGTTCACCCACTACTGTGATTTATGAGGATAAC
CAAAGACCCTCTGGGGTCCCTGATCGGTTTTCTGGCTCCATCGACAGCAGTTCC
AATTCGGCCTCCCTCACCATCTCTGGACTAAAAACTGAGGACGAGGCTGACTA
CTACTGTCAGTCCTGGGACCCCGTGTTCGGGGTGTTCGGCGGAGGGACAAAGC
TGACCGTCTTAGGGGCGGCC
SEQ ID NO: 2-anti-PTPRZ1 scFv “RRB470” nucleotide sequence
ATGGCACAAGTGCAGTTAGTTCAGTCTGGGGCTGAAGTGAAGAAGCCTGGGTC
CTCGGTGAAGGTCTCCTGCAAGGCTAGTGGAGGCACCTTCAGCAGCTATGCTA
TCAGCTGGGTGCGACAGGCCCCTGGACAAGGCCTTGAATGGATGGGAGGGATC
ATTCCGATCTTTGGTACAGCAAACTACGCACAGAAGTTCCAAGGCAGAGTCAC
TATTACCGCGGACGAATCCACAAGCACAGCATACATGGAGCTGAGCAGCCTGA
GGTCTGAAGATACGGCCGTGTATTACTGTGCGAGAGAGGGGGGGGCCGTGGG
GTACTACTACGGTATGGACGTCTGGGGCCAGGGAACACTTGTGACAGTCTCCA
GCGGTGGAGGCGGTTCAGGCGGAGGCGGCTCAGGCGGTGGCGGATCTACTCA
GAGTGCCTTGACTCAGCCAGCCTCCGTGTCTGGGTCACCCGGACAGTCGATAA
CCATCTCCTGCACTGGAACCAGCAGTGACGTTGGTGGTTATAATTATGTATCCT
GGTACCAACAGCACCCAGGCAAAGCCCCCAAACTCATGATTTATGAGGTCAGT
AATCGGCCCTCAGGGGTTTCTAATCGTTTCTCTGGCTCCAAATCTGGCAACACG
GCCTCCCTGACTATATCAGGGCTCCAGGCTGAGGACGAGGCTGATTATTACTG
TAGTTCATATGATAGGAGCAACCGCAGTATGGTGTTTGGCGGAGGGACCAAAC
TGACCGTACTAGGGGCAGCC
SEQ ID NO: 3-anti-PTPRZ1 scFv “RRB471” nucleotide sequence
ATGGCCGAAGTGCAGCTGGTGGAGTCTGGGGGAGGCTTGGTACAGCCTGGGG
GGTCCCTGCGTCTCTCCTGTGCAGCCTCTGGATTTACCTTCAGTAGCTATAGCA
TGAACTGGGTCAGGCAGGCTCCAGGGAAGGGGCTTGAGTGGGTTTCATACATT
AGTAGCAGTAGTAGCACAATATACTACGCAGACTCTGTGAAGGGCCGATTCAC
AATCTCCAGGGATAATGCCAAGAACTCACTGTATTTACAAATGAATAGCCTTA
GAGCCGAAGACACGGCTGTGTATTACTGTGCGAGACCAGGCTACGGTGACTTT
CCCGGTGCTTTTGATATCTGGGGCCAGGGAACCCTGGTCACAGTGTCGAGCGG
TGGAGGCGGCTCAGGCGGAGGTGGCAGCGGCGGTGGCGGATCTACGCAGTCT
GCATTGACTCAGCCTGCCTCCGTGTCTGGATCACCTGGACAAAGCATTACCATC
TCCTGCACTGGAACCAGCAGTGACGTTGGTGGTTATAACTATGTCTCCTGGTAC
CAACAGCACCCGGGCAAAGCCCCCAAACTCATGATTTACGAAGTAAGTAATCG
GCCCTCAGGGGTTTCTAATCGCTTCTCTGGCTCCAAATCCGGCAACACTGCCTC
CCTGACAATCAGTGGGCTCCAGGCTGAGGACGAGGCTGATTATTACTGCAGCT
CATATGATTGGGCCACCTACGGGTCGGTGTTCGGCGGAGGGACCAAGCTGACT
GTCCTAGGGGCGGCA
SEQ ID NO: 4-anti-PTPRZ1 scFv “RRB473” nucleotide sequence
ATGGCCCAAGTGCAGCTGGTGGAGTCTGGGGGAGGCGTTGTCCAGCCTGGGAG
GTCACTGAGACTCTCCTGTGCAGCCTCTGGGTTTACATTCAGTAGCTATGCTAT
GCACTGGGTACGCCAAGCTCCTGGCAAAGGGCTGGAGTGGGTGGCAGTTATAT
CATATGATGGTAGTAACAAATACTACGCAGACAGTGTGAAGGGCCGATTCACC
ATCTCCAGAGACAATTCCAAGAACACTCTTTATCTGCAAATGAACAGCCTGCG
GGCTGAAGACACAGCTGTGTACTACTGTGCGCGGGACCAGGATGACTCCAGTG
ATGCTTTTGATATCTGGGGCCAGGGAACCCTGGTCACAGTCTCGAGCGGTGGA
GGCGGTAGCGGCGGAGGTGGCAGCGGCGGTGGCGGATCGACGCAGAGCGTTT
TGACGCAACCGCCCTCAGTGTCTGCAGCCCCAGGACAGAAAGTCACCATATCC
TGCTCTGGAAGCAGCTCCAACATTGGGAACAATTATGTATCCTGGTACCAGCA
GTTGCCAGGGACAGCCCCCAAACTCCTCATTTACGACAATAATAAGCGTCCCT
CAGGGATTCCTGACCGCTTTTCTGGCAGTAAGTCTGGCACTTCAGCCACTCTGG
GCATCACCGGACTTCAGACTGGGGACGAAGCCGATTATTACTGCGGAACATAT
GATTACATCGCGACCAGGGCCGTGTTCGGTGGCGGGACCAAGTTAACTGTGCT
AGGGGCAGCC
SEQ ID NO: 5-anti-PTPRZ1 scFv “RRB474” nucleotide sequence
ATGGCCCAGGTGCAGCTTGTTCAGTCTGGGGCTGAGGTGAAGAAGCCAGGGTC
CTCGGTGAAGGTTTCCTGCAAGGCTTCAGGAGGCACCTTCAGCAGCTATGCTA
TCAGTTGGGTGCGGCAAGCACCTGGCCAAGGGCTTGAGTGGATGGGAGGGAT
AATCCCTATCTTTGGTACAGCAAACTACGCACAAAAGTTCCAGGGCCGCGTCA
CGATTACCGCCGACGAATCCACCAGCACAGCCTACATGGAACTGTCCAGCCTG
AGAAGTGAAGACACTGCCGTGTATTACTGTGCGCGTGGGACGTATTACGATTT
TTGGAGTGGTTATTATGATGCTTTTGATATCTGGGGCCAGGGAACCCTGGTTAC
AGTCTCTAGCGGTGGAGGCGGTTCAGGCGGAGGTGGCAGCGGCGGTGGCGGC
TCTACTCAGTCTGTGTTGACACAGCCGCCCTCAGTGTCTGCAGCCCCAGGACA
GAAGGTCACCATCTCCTGTTCTGGTAGCAGCTCCAACATTGGGAATAATTACGT
ATCCTGGTACCAGCAGTTGCCAGGAACAGCCCCCAAACTCTTAATATATGACA
ATAACAAAAGGCCCTCAGGGATTCCTGACCGATTCAGTGGCTCCAAATCTGGC
ACTTCAGCTACCCTGGGCATTACCGGACTCCAGACTGGGGACGAGGCCGATTA
TTACTGCGGAACATGGGATAGTTCGTACTGGCAACCCGTATTCGGCGGAGGGA
CTAAACTGACCGTCCTAGGGGCGGCC
SEQ ID NO: 6-anti-PTPRZ1 scFv “RRB476” nucleotide sequence
ATGGCCCAGGTGCAACTGGTGGAATCTGGGGGAGGCGTGGTCCAGCCTGGGA
GGTCGTTGCGGCTCTCCTGTGCAGCCTCTGGATTCACCTTTAGTAGCTATGCTA
TGCACTGGGTCCGCCAAGCTCCAGGCAAGGGGCTGGAATGGGTGGCAGTTATA
TCATACGATGGAAGTAATAAATACTACGCAGACTCCGTGAAGGGCCGCTTCAC
CATCTCCAGAGACAATTCCAAGAACACACTGTATCTGCAAATGAACAGCCTGA
GGGCTGAGGACACTGCTGTGTATTACTGTGCGAGAGACCAGGATGACTCCAGT
GATGCTTTTGATATCTGGGGGCAGGGAACCCTGGTAACAGTCAGTAGCGGTGG
AGGCGGTTCAGGCGGCGGTGGCAGCGGCGGTGGCGGTAGCACGCAGTCTGTGT
TGACGCAGCCGCCCTCAGTGTCTGCAGCCCCAGGACAAAAGGTCACTATCTCC
TGCTCTGGAAGCAGCTCCAACATTGGGAACAACTATGTATCCTGGTACCAGCA
GTTACCTGGTACAGCCCCCAAACTCCTCATTTATGACAATAATAAGCGACCCTC
AGGGATTCCTGACCGTTTTAGTGGCAGCAAATCTGGCACTTCAGCCACCCTTGG
CATCACCGGACTCCAGACTGGGGACGAGGCCGATTATTACTGCGGAACATGGG
ATTACAAAGTTTCGCGGCTTGTCTTCGGCGGAGGGACCAAGCTGACAGTTCTA
GGGGCGGCC
SEQ ID NO: 7-anti-CSPG4 scFv “HRB298” nucleotide sequence
ATGGCCCAGGTGCAGCTGGTGCAGTCTGGAGCTGAGGTGAAGAAGCCTGGGG
CCTCAGTTAAAGTTTCCTGTAAGGCTAGTGGTTACACCTTTACCAGCTATGGTA
TCAGTTGGGTGAGGCAGGCCCCTGGACAAGGGCTTGAATGGATGGGATGGATC
AGCGCTTACAATGGTAACACAAACTATGCACAGAAGCTCCAGGGCAGAGTCAC
CATGACCACAGACACATCCACTAGCACAGCATACATGGAGCTGAGGAGTTTGA
GATCTGACGACACGGCCGTTTATTACTGTGCGCGGCGAGATTACTATGATGGT
AGTGGATTTGACTACTGGGGCCAAGGAACTCTGGTCACAGTCTCGAGCGGTGG
AGGCGGTTCCGGCGGAGGGGGCAGCGGCGGTGGCGGATCAACTCAGTCTGTGT
TGACGCAGCCGCCCTCAGTGAGTGCAGCCCCAGGCCAAAAAGTCACCATCTCC
TGCTCTGGAAGCAGCTCCAACATTGGGAATAATTACGTATCCTGGTACCAGCA
GCTCCCAGGCACAGCTCCCAAATTACTTATTTATGATAATAACAAGCGTCCCTC
AGGGATTCCTGACCGGTTCTCTGGCTCCAAATCTGGCACGTCAGCCACCCTGG
GCATAACTGGGCTCCAAACTGGGGACGAAGCCGATTATTACTGCGGAACTTAT
GATGGCGAAGGGCGCCACGAGGTGTTCGGCGGAGGGACCAAGCTGACCGTAC
TAGGGGCGGCA
SEQ ID NO: 8-anti-CSPG4 scFv “HRB299” nucleotide sequence
ATGGCCCAGGTGCAACTGGTGGAATCGGGCGGAGGCGTGGTCCAGCCTGGGA
GGTCCCTGAGACTTTCCTGTGCAGCCTCTGGATTTACCTTCAGTAGCTATGCTA
TGCACTGGGTGCGCCAAGCTCCAGGCAAGGGGCTGGAATGGGTTGCAGTTATA
TCATATGATGGAAGTAATAAATACTACGCAGACTCCGTGAAGGGCCGTTTTAC
CATCTCCCGGGACAATTCCAAAAACACTCTGTATCTGCAAATGAACAGCCTTA
GAGCTGAAGACACTGCCGTGTACTACTGCGCGCGCGATCCGTGGGGTGGTTGG
TTAGGGAGCGATGCTTTTGACATTTGGGGCCAAGGAACCTTGGTCACAGTCTC
GAGCGGTGGAGGCGGTTCAGGCGGAGGTGGCAGCGGCGGTGGCGGCAGCACG
CAGTCTGTGCTGACTCAGCCACCCTCAGCGTCTGGGACACCCGGGCAGAGGGT
CACCATTTCTTGTTCTGGAAGCAGCAGCAACATTGGAAGTAACACTGTAAACT
GGTACCAGCAGTTGCCAGGAACGGCCCCCAAACTCCTCATCTATAGTAATAAT
CAGCGGCCTTCAGGGGTACCTGACCGATTCTCCGGCTCCAAGTCTGGCACCTC
AGCCTCCCTCGCCATCAGTGGGCTCCAGTCTGAGGATGAGGCTGATTATTACTG
CGCAGCATACGATGGGGACGGGGGGGAGGACGTGTTCGGCGGAGGTACAAAG
CTGACAGTTCTAGGGGCCGCC
SEQ ID NO: 9-anti-CSPG4 scFv “HRB300” nucleotide sequence
ATGGCCGAGGTGCAGCTGTTGGAATCTGGGGGAGGCTTGGTACAGCCGGGGG
GGTCCCTGCGTCTCTCCTGTGCAGCCTCTGGATTCACCTTTAGCAGCTATGCTA
TGAGCTGGGTGCGCCAGGCTCCAGGGAAGGGGCTGGAATGGGTGTCAGCTATT
AGTGGTAGTGGTGGCAGCACATACTACGCAGATTCCGTGAAAGGCCGGTTCAC
AATCTCCAGAGATAATAGTAAGAACACACTGTACCTTCAAATGAACAGCTTAC
GCGCCGAGGACACGGCGGTGTATTACTGTGCAAGACGATATAGCAGTGGCTGG
TCATACTACTTTGACTACTGGGGCCAGGGAACCCTGGTCACAGTCTCGAGCGG
TGGAGGCGGTTCAGGCGGAGGTGGCAGCGGCGGTGGCGGATCTACGCAGTCT
GCCCTGACTCAACCTGCCTCCGTGTCTGGGTCTCCTGGACAGTCGATCACCATA
TCCTGCACTGGAACATCCAGTGACGTTGGAGGTTATAACTATGTTTCCTGGTAC
CAACAGCACCCAGGCAAAGCCCCCAAACTCATGATTTATGAAGTAAGTAATCG
GCCCTCAGGGGTTTCTAATAGGTTCTCAGGCTCCAAGAGTGGCAACACTGCCT
CCCTGACCATCTCTGGGCTCCAGGCTGAGGACGAGGCTGATTACTACTGCAGC
TCATATGATACTTTTGAGAGGATTAGCGTGTTCGGCGGAGGGACCAAGCTTAC
CGTCCTAGGGGCGGCA
SEQ ID NO: 10-anti-CSPG4 scFv “HRB301” nucleotide sequence
ATGGCACAAGTGCAACTGGTGCAGTCTGGAGCTGAAGTGAAGAAACCGGGGG
CCTCAGTTAAGGTCTCCTGCAAAGCTTCTGGTTACACCTTTACTAGCTATGGTA
TCAGCTGGGTGAGACAAGCCCCTGGACAAGGGCTTGAGTGGATGGGCTGGATC
AGCGCTTACAATGGTAACACAAACTATGCACAGAAGCTCCAGGGCCGTGTCAC
CATGACCACAGACACATCCACCTCCACAGCCTACATGGAGCTGAGGAGCCTGA
GATCGGACGACACGGCTGTGTATTACTGTGCGAGACGGAGTTATGATAGTAGT
GGACTTGACTACTGGGGCCAGGGAACATTGGTTACAGTTTCGAGCGGCGGCGG
CGGTTCAGGCGGAGGGGGCAGCGGCGGTGGCGGTTCTACGCAGTCCGTGTTGA
CTCAGCCACCCTCAGCGTCTGGGACCCCCGGGCAGAGGGTCACCATTTCTTGTT
CTGGAAGCAGCTCCAACATTGGAAGTAATACTGTAAACTGGTACCAGCAGCTC
CCAGGAACTGCCCCTAAATTACTCATATATAGTAATAATCAGCGGCCCTCAGG
GGTACCTGACCGATTCTCCGGCTCCAAGTCTGGCACCTCAGCCTCCCTGGCCAT
CAGTGGGCTGCAGAGCGAAGATGAGGCTGATTATTACTGCGCAGCATGGGATC
GCAGGTGGCGCCTGGTGTTCGGCGGAGGGACCAAGCTGACTGTCCTAGGGGCC
GCC
SEQ ID NO: 11-anti-CSPG4 scFv “HRB302” nucleotide sequence
ATGGCCCAGGTGCAGCTTGTGCAGTCCGGTGCTGAGGTGAAGAAACCTGGGGC
CTCAGTTAAGGTTTCCTGCAAGGCTTCTGGTTACACCTTTACCAGCTATGGTAT
CAGCTGGGTGCGACAGGCCCCTGGACAAGGGCTTGAGTGGATGGGCTGGATA
AGCGCTTACAATGGTAACACAAACTATGCACAAAAGTTGCAGGGCCGTGTCAC
CATGACCACAGACACATCCACAAGCACAGCCTACATGGAGCTGAGGTCCTTGA
GATCTGACGACACGGCCGTGTATTACTGCGCAAGACAGGTGGGCGCTCCGACT
CGCTTTGACTACTGGGGCCAGGGAACCCTGGTGACAGTCTCGAGCGGTGGAGG
CGGGTCAGGCGGAGGCGGCAGCGGCGGTGGCGGATCGACGCAGTCTGTGCTG
ACTCAACCACCATCAGCGTCCGGGACCCCCGGGCAAAGGGTCACTATTAGTTG
TAGTGGAAGCAGCTCCAACATTGGAAGTAATACTGTAAACTGGTACCAGCAGC
TCCCAGGAACTGCCCCCAAACTCTTAATCTATAGTAATAATCAGCGGCCCTCA
GGGGTTCCTGATCGGTTCTCTGGCTCCAAGTCTGGCACCTCAGCCTCCCTGGCA
ATCAGTGGGCTCCAGTCTGAAGATGAAGCTGATTATTACTGTGCAGCATGGGA
TACGCACGCCTGGGCCCCCGTATTCGGCGGAGGGACTAAACTGACCGTCCTAG
GGGCGGCT
SEQ ID NO: 12-anti-CSPG4 scFv “HRB303” nucleotide sequence
ATGGCCCAGGTGCAACTGGTGCAGTCTGGGGCTGAGGTCAAGAAGCCAGGGTC
CTCGGTGAAGGTCTCCTGTAAGGCTAGTGGAGGCACCTTCAGCAGCTATGCTA
TCAGCTGGGTGAGACAAGCCCCTGGACAAGGGCTTGAGTGGATGGGAGGGAT
CATCCCTATATTTGGTACAGCAAACTACGCACAGAAATTTCAGGGCAGAGTTA
CGATAACTGCAGACGAATCCACTAGCACAGCATACATGGAGCTGAGTAGTTTA
AGGTCTGAAGACACTGCAGTGTATTACTGTGCTCGTTCTAAATATAACTGGGCC
TACAAAAATGATTACTGGGGCCAGGGAACCCTGGTTACAGTTTCAAGCGGTGG
AGGCGGTTCAGGCGGAGGTGGCAGCGGCGGTGGCGGCTCTACACAGAGCGTG
TTGACGCAGCCGCCCTCAGTATCGGCGGCCCCAGGGCAGAAGGTCACCATCTC
CTGCTCTGGAAGCAGTTCCAACATTGGGAATAACTATGTATCCTGGTACCAGC
AGCTCCCAGGTACAGCCCCCAAATTGCTCATTTACGACAATAATAAGCGACCC
TCAGGGATTCCTGATCGCTTCAGTGGCTCCAAATCTGGCACCTCAGCCACCCTG
GGCATCACCGGACTTCAAACTGGGGACGAAGCTGATTATTACTGCGGAACATA
TGATCCCTGGGCTCGGACTGCCGTGTTCGGCGGAGGGACCAAGCTGACCGTCC
TAGGGGCGGCC
SEQ ID NO: 13-anti-CSPG4 scFv “225.28S” nucleotide sequence
CAGGTTAAACTCCAACAAAGTGGCGGAGGCTTGGTTCAGCCTGGAGGGAGTAT
GAAACTGTCTTGTGTCGTATCTGGTTTTACATTCTCAAATTATTGGATGAATTG
GGTTAGGCAATCACCGGAGAAGGGATTGGAATGGATCGCTGAGATTCGGTTGA
AATCAAACAACTTCGGTCGCTATTATGCGGAATCCGTGAAAGGTCGGTTCACG
ATTTCCCGCGACGATTCAAAGTCCAGTGCTTATCTGCAAATGATTAATCTTCGG
GCAGAAGATACAGGAATATACTATTGTACCTCCTATGGTAACTATGTTGGTCA
CTATTTCGATCATTGGGGGCAGGGAACCACTGTCACCGTATCCAGCggtggcggagg
gagcggcggtggaggaagcggaggcggaggttccGACATTGAACTGACTCAATCTCCCAAATTTA
TGTCAACGAGCGTCGGGGACCGCGTGAGCGTTACGTGTAAGGCTTCACAAAAC
GTAGACACCAATGTGGCCTGGTATCAACAAAAGCCGGGACAATCTCCAGAGCC
CCTGCTCTTTTCAGCAAGTTACAGGTACACCGGTGTTCCAGATAGATTCACAGG
TAGTGGATCTGGTACTGATTTTACTCTCACCATAAGTAACGTGCAGTCCGAAGA
CCTCGCCGAGTACTTTTGTCAACAGTATAATAGTTACCCACTTACATTTGGGGG
TGGAACAAAACTGGAAATCAAG
SEQ ID NO: 14-anti-BCAN scFv “HRB294” nucleotide sequence
ATGGCCCAGGTACAACTGCAACAGTCAGGGCCAGGACTGGTTAAGCCCTCGCA
GACCTTATCACTTACCTGTGCCATCTCCGGGGACAGTGTCTCTAGCAACAGTGC
TGCTTGGAACTGGATAAGGCAGTCCCCAAGCCGCGGCCTTGAATGGCTGGGAA
GAACATACTACAGGTCCAAGTGGTATAATGATTATGCAGTTAGTGTGAAAAGT
CGAATAACTATCAACCCTGATACATCCAAGAACCAGTTCTCCCTGCAGCTGAA
CTCTGTGACTCCTGAGGACACAGCTGTGTATTACTGTGCGAGAGATCAGCGGA
ATTACGATTTTTGGAGTGGTTATTATCCGCCCGCAGAATTAGGGTACTACGGTA
TGGACGTCTGGGGCCAGGGAACCCTGGTCACAGTCTCGAGCGGTGGAGGCGGT
TCAGGCGGAGGCGGCAGCGGCGGTGGCGGCTCTACGCAGTCTGTGTTGACGCA
GCCGCCTAGCGTGTCTGCTGCCCCAGGTCAGAAAGTGACCATCTCCTGCTCTG
GAAGCAGCTCCAACATTGGGAATAATTATGTATCCTGGTACCAGCAACTCCCA
GGAACAGCACCCAAACTCCTCATTTATGACAATAATAAGCGGCCCTCAGGGAT
TCCTGACCGTTTTTCTGGCAGTAAAAGCGGCACTTCAGCCACTCTGGGCATCAC
CGGGCTCCAAACTGGGGACGAGGCCGATTACTACTGCGGAACATGGGATTGGA
GCGCATTGGTGGTGTTCGGCGGAGGGACCAAGCTGACCGTTCTAGGGGCGGCC
SEQ ID NO: 15-anti-BCAN scFv “HRB295” nucleotide sequence
ATGGCCCAGGTGCAGCTGGTGGAGTCTGGGGGTGGCGTGGTCCAGCCTGGGAG
GTCCTTGCGTCTCTCCTGCGCAGCCTCTGGATTCACTTTCAGTAGCTATGCTAT
GCACTGGGTCCGCCAAGCTCCGGGCAAGGGGCTGGAATGGGTGGCAGTTATAA
GCTATGATGGAAGTAATAAATACTACGCAGACTCCGTGAAGGGCCGATTCACC
ATCTCCAGAGACAATTCCAAGAACACGCTTTATCTGCAAATGAACAGCCTTCG
CGCTGAAGACACAGCTGTGTATTACTGTGCCAGAGTATCAGACTGGAACGACG
CCGCTTTTGATATTTGGGGCCAGGGAACTCTGGTTACAGTCTCGAGCGGTGGA
GGCGGTTCAGGCGGAGGTGGCAGCGGCGGTGGCGGATCGACCCAGTCTGTGCT
GACTCAGCCACCCTCAGCGAGCGGGACACCAGGGCAGCGGGTCACCATTTCTT
GTTCTGGAAGCAGCTCCAACATCGGTAGTAATACTGTAAACTGGTACCAGCAA
CTGCCAGGAACGGCCCCCAAACTCCTCATCTACAGTAATAATCAACGGCCTTC
AGGGGTTCCTGATAGATTTTCTGGCTCCAAAAGCGGCACCTCAGCCTCCCTGGC
CATTAGTGGGTTACAGTCTGAAGATGAGGCTGATTATTACTGCGCAGCATGGG
ACCCCGAGACCGCCAGGTGGGTGTTTGGCGGAGGGACAAAGTTGACCGTCCTA
GGGGCGGCC
SEQ ID NO: 16-anti-BCAN scFv “HRB296” nucleotide sequence
ATGGCCCAGGTACAGTTGCAGCAATCAGGTCCAGGACTGGTGAAGCCCAGCCA
AACCTTATCATTAACTTGTGCAATCTCCGGGGACAGTGTTTCTAGCAACAGTGC
TGCTTGGAACTGGATCAGGCAGTCCCCTTCGAGAGGCCTTGAGTGGCTGGGAA
GGACATACTACCGGAGCAAGTGGTATAATGATTATGCAGTTAGCGTGAAAAGT
CGGATAACCATCAACCCTGACACATCCAAGAACCAGTTCTCCCTGCAACTGAA
CTCTGTGACTCCCGAAGACACGGCTGTGTATTACTGTGCACGCAGAGGGGAAC
ACTATGATAGTAGTGGTTATTACTACGGCCTTGATTACTGGGGCCAGGGAACC
CTGGTCACAGTCAGCAGCGGTGGAGGCGGTTCAGGCGGCGGTGGCAGCGGCG
GTGGCGGATCTACGCAGTCGGTGTTGACACAGCCGCCCTCAGTGTCTGCGGCC
CCAGGGCAAAAAGTTACCATATCCTGCTCTGGAAGCAGCTCCAACATTGGGAA
TAATTATGTATCCTGGTACCAGCAGCTCCCAGGAACAGCCCCCAAACTCCTCA
TTTATGACAATAATAAGCGTCCTTCAGGGATTCCTGACCGATTCTCTGGCTCCA
AGTCTGGCACTTCAGCCACTCTGGGCATCACCGGACTCCAGACTGGGGACGAG
GCCGATTACTACTGCGGAACATATGATGTCGCGGCTGGGTACGTGTTTGGCGG
AGGGACCAAACTGACCGTCCTAGGGGCAGCC
SEQ ID NO: 17-anti-BCAN scFv “HRB297” nucleotide sequence
ATGGCCCAGGTACAGCTGGTGCAGAGCGGGGCTGAGGTGAAAAAGCCCGGGT
CCTCGGTGAAAGTGTCCTGCAAGGCTTCTGGAGGCACCTTCAGCAGCTATGCT
ATAAGCTGGGTGCGACAAGCCCCGGGACAAGGGCTTGAGTGGATGGGAGGGA
TCATCCCTATCTTTGGTACAGCAAACTACGCACAGAAGTTCCAGGGCAGAGTC
ACCATTACCGCGGACGAATCCACGAGCACAGCCTACATGGAACTGAGCAGCCT
TAGGTCTGAGGACACTGCCGTGTATTACTGTGCGAGACCACGTACTGCAGGCT
GGAGTTATGATGCTTTTGATGCCTGGGGCCAGGGAACATTGGTAACAGTCTCA
AGCGGTGGAGGCGGTTCAGGCGGAGGTGGCAGCGGCGGTGGCGGATCTACGC
AATCTGCCTTAACTCAGCCTGCCAGTGTGTCTGGGAGTCCTGGACAGTCAATA
ACCATTTCCTGTACTGGAACCAGCAGTGACGTTGGCGGTTATAACTACGTTTCC
TGGTACCAACAGCACCCAGGCAAAGCCCCCAAACTCATGATTTACGAAGTCAG
TAATAGGCCCTCAGGGGTTTCTAATCGCTTCTCTGGCTCCAAGTCAGGCAACAC
AGCCTCCCTGACTATCTCTGGGCTCCAGGCTGAGGACGAGGCTGATTATTACTG
CAGTTCATATGATTGGCGGTCCTCCGGGTCGGTGTTTGGCGGAGGGACCAAGC
TGACCGTCCTAGGGGCAGCA
SEQ ID NO: 18-anti-TNC scFv “E10” nucleotide sequence
GAGGTACAGCTAGTGGAGTCAGGCGGGGGCCTGGTCCAGCCAGGCGGATCGTT
AAGACTTAGTTGCGCAGCAAGCGGGTTTACGTTCTCAGGTAGCCGAATGGGGT
GGGTGAGACAGGCCCCCGGGAAAGGACTCGAATGGGTTTCCGCGATCAACGA
AGAAGGTGGACAAACTTACTACGCCGATAGCGTGAAGGGACGGTTTACAATTT
CTCGTGACAACTCCAAGAATACCCTGTATCTGCAAATGAATAGTTTGAGGGCT
GAGGACACCGCCGTCTATTATTGTGCTAAACATCCTCCGCACCGCCCCTTCGAT
TACTGGGGCCAGGGCACACTCGTGACTGTTTCTAGGGGTGGAGGCGGTTCCGG
CGGAGGGGGCAGCGGCGGTGGCGGATCATCTTCCGAATTAACTCAAGACCCAG
CCGTTTCTGTGGCCCTCGGCCAGACAGTCAGGATCACGTGCCAAGGGGATAGT
CTGCGATCCTACTATGCAAGCTGGTACCAGCAGAAACCGGGCCAGGCTCCTGT
ATTGGTGATCTACGGAAAGAATAACAGACCCTCTGGTATACCCGACCGGTTCT
CCGGTAGCAGTAGCGGCAACACCGCTTCACTTACTATTACCGGAGCACAGGCC
GAGGATGAGGCGGACTATTATTGTAATTCATCGCACGGCCCACGTCGCCCTGT
CGTGTTTGGGGGAGGCACCAAGCTGACAGTGCTGGGG
SEQ ID NO: 19-anti-TNC scFv “P12” nucleotide sequence
GAGGTGCAGCTGGTCGAGAGCGGTGGAGGCCTCGTGCAGCCCGGAGGGTCATT
GCGTCTCTCCTGCGCCGCCAGCGGCTTTACCTTCGGTCAATATAGCATGAGTTG
GGTCAGGCAGGCGCCTGGCAAGGGACTGGAATGGGTTTCCGCCATCACCGGTA
CAGGAGGGGAAACATACTACGCTGACTCAGTAGAGGGGAGATTCACTATTTCT
CGAGATAACTCCAAGAACACGCTGTATCTACAAATGAATTCTTTACGCGCAGA
AGACACTGCAGTTTATTACTGTGCTAAAGGGAGACGGATATTTGATTACTGGG
GCCAGGGCACCCTTGTGACAGTGAGTAGGGGTGGAGGCGGTTCCGGCGGAGG
GGGCAGCGGCGGTGGCGGATCATCCTCCGAACTGACACAGGACCCGGCTGTG
AGCGTCGCCCTTGGTCAGACTGTGAGAATTACATGCCAGGGGGATTCACTCAG
GCGGCAGCCTGCTTCTTGGTACCAACAAAAGCCTGGACAGGCCCCCGTGTTGG
TGATATACTACAAAAAGCTGCGACCATCAGGAATCCCTGACCGCTTTTCTGGA
AGCTCCAGTGGGAATACCGCAAGTCTCACCATCACTGGTGCCCAGGCAGAGGA
TGAGGCGGACTATTATTGTAACTCGTTCAGCCCCAAACCCAAACCAGTAGTCTT
CGGCGGCGGGACCAAGCTGACGGTTTTAGGC
SEQ ID NO: 20-anti-TNC scFv “F16” nucleotide sequence
GAGGTCCAATTACTTGAATCAGGCGGAGGCCTGGTGCAGCCTGGAGGCAGCCT
GAGACTGTCCTGCGCGGCAAGCGGTTTCACTTTCTCCAGATATGGCGCATCCTG
GGTTAGGCAGGCACCCGGTAAAGGACTGGAGTGGGTATCTGCCATTTCTGGGA
GTGGAGGGAGTACCTACTACGCTGATTCGGTGAAGGGGCGTTTTACAATCTCA
CGAGACAATAGCAAAAACACACTATATCTCCAGATGAATTCTCTCCGCGCCGA
AGACACGGCTGTCTACTATTGTGCTAAGGCCCACAACGCCTTTGATTACTGGG
GCCAGGGGACCTTGGTGACTGTGAGCCGGGGTGGAGGCGGTTCCGGCGGAGG
GGGCAGCGGCGGTGGCGGATCATCGAGCGAACTGACACAGGACCCGGCGGTT
TCCGTGGCACTGGGGCAGACAGTAAGAATAACTTGTCAAGGGGATAGCCTGCG
CAGTTACTACGCCAGCTGGTACCAGCAGAAACCAGGCCAGGCCCCCGTTTTGG
TGATTTATGGGAAGAATAACAGGCCTTCCGGCATCCCCGACCGGTTTTCTGGAT
CTAGTTCTGGAAACACCGCATCACTTACCATCACGGGAGCTCAAGCCGAGGAT
GAGGCTGACTACTATTGCAATTCATCCGTCTATACTATGCCTCCAGTGGTGTTC
GGTGGCGGTACAAAGTTAACCGTCCTCGGC

All of SEQ ID NO: 21-40 are scFv in orientation VH-linker-VL with the linker (G4S) 3 in lowercase. Antigen binding regions are highlighted in underlined (ABR1), bold and underlined (ABR2) and underlined and italicised (ABR3). All ABRs are predicted using Paratome (Kunik V et al (2012). Structural Consensus among Antibodies Defines the Antigen Binding Site. PLOS Comput Biol 8 (2): e1002388. doi:10.1371/journal.pcbi. 1002388; and Kunik V et al (2012). Paratome: An online tool for systematic identification of antigen binding regions in antibodies based on sequence or structure. Nucleic Acids Res. 2012 July; 40 (Web Server issue): W521-4. doi: 10.1093/nar/gks480. Epub 2012 Jun. 6).

anti-PTPRZ1 scFv “RRB469” polypeptide sequence
SEQ ID NO: 21
MAQVQLVESGGGVVQPGRSLRLSCAASGFTFSSYAMHWVRQAPGKGLEWVAVI
SYDGSNKYYADSVKGRFTISRDNSKNTLYLQMNSLRAEDTAVYYCARGSGYSYGP
GYDAFDIWGQGTLVTVSSggggsggggsggggsTNFMLTQPHSVSESPGKTVTISCTRSSG
SIASNYVQWYQQRPGSSPTTVIYEDNQRPSGVPDRFSGSIDSSSNSASLTISGLKTE
DEADYYCQSWDPVFGVFGGGTKLTVLGAA
anti-PTPRZ1 scFv “RRB470” polypeptide sequence
SEQ ID NO: 22
MAQVQLVQSGAEVKKPGSSVKVSCKASGGTFSSYAISWVRQAPGQGLEWMGGII
PIFGTANYAQKFQGRVTITADESTSTAYMELSSLRSEDTAVYYCAREGGAVGYYYG
MDVWGQGTLVTVSSggggsggggsggggsTQSALTQPASVSGSPGQSITISCTGTSSDVG
GYNYVSWYQQHPGKAPKLMIYEVSNRPSGVSNRFSGSKSGNTASLTISGLQAEDE
ADYYCSSYDRSNRSMVFGGGTKLTVLGAA
anti-PTPRZ1 scFv “RRB471” polypeptide sequence
SEQ ID NO: 23
MAEVQLVESGGGLVQPGGSLRLSCAASGFTFSSYSMNWVRQAPGKGLEWVSYIS
SSSSTIYYADSVKGRFTISRDNAKNSLYLQMNSLRAEDTAVYYCARPGYGDFPGA
FDIWGQGTLVTVSSggggggggsggggsTQSALTQPASVSGSPGQSITISCTGTSSDVGG
YNYVSWYQQHPGKAPKLMIYEVSNRPSGVSNRFSGSKSGNTASLTISGLQAEDEA
DYYCSSYDWATYGSVFGGGTKLTVLGAA
anti-PTPRZ1 scFv “RRB473” polypeptide sequence
SEQ ID NO: 24
MAQVQLVESGGGVVQPGRSLRLSCAASGFTFSSYAMHWVRQAPGKGLEWVAVI
SYDGSNKYYADSVKGRFTISRDNSKNTLYLQMNSLRAEDTAVYYCARDQDDSSD
AFDIWGQGTLVTVSSggggsggggsggggsTQSVLTQPPSVSAAPGQKVTISCSGSSSNIG
NNYVSWYQQLPGTAPKLLIYDNNKRPSGIPDRFSGSKSGTSATLGITGLQTGDEA
DYYCGTYDYIATRAVFGGGTKLTVLGAA
anti-PTPRZ1 scFv “RRB474” polypeptide sequence
SEQ ID NO: 25
MAQVQLVQSGAEVKKPGSSVKVSCKASGGTFSSYAISWVRQAPGQGLEWMGGII
PIFGTANYAQKFQGRVTITADESTSTAYMELSSLRSEDTAVYYCARGTYYDEWSGY
YDAFDIWGQGTLVTVSSggggsggggsggggsTQSVLTQPPSVSAAPGQKVTISCSGSSSN
IGNNYVSWYQQLPGTAPKLLIYDNNKRPSGIPDRFSGSKSGTSATLGITGLQTGDE
ADYYCGTWDSSYWQPVFGGGTKLTVLGAA
anti-PTPRZ1 scFv “RRB476” polypeptide sequence
SEQ ID NO: 26
MAQVQLVESGGGVVQPGRSLRLSCAASGFTFSSYAMHWVRQAPGKGLEWVAVI
SYDGSNKYYADSVKGRFTISRDNSKNTLYLQMNSLRAEDTAVYYCARDQDDSSD
AFDIWGQGTLVTVSSggggsggggsggggsTQSVLTQPPSVSAAPGQKVTISCSGSSSNIG
NNYVSWYQQLPGTAPKLLIYDNNKRPSGIPDRFSGSKSGTSATLGITGLQTGDEA
DYYCGTWDYKVSRLVFGGGTKLTVLGAA
anti-CSPG4 scFv “HRB298” polypeptide sequence
SEQ ID NO: 27
MAQVQLVQSGAEVKKPGASVKVSCKASGYTFTSYGISWVRQAPGQGLEWMGWI
SAYNGNTNYAQKLQGRVTMTTDTSTSTAYMELRSLRSDDTAVYYCARRDYYDGS
GFDYWGQGTLVTVSSggggsggggsggggsTQSVLTQPPSVSAAPGQKVTISCSGSSSNIG
NNYVSWYQQLPGTAPKLLIYDNNKRPSGIPDRFSGSKSGTSATLGITGLQTGDEA
DYYCGTYDGEGRHEVFGGGTKLTVLGAA
anti-CSPG4 scFv “HRB299” polypeptide sequence
SEQ ID NO: 28
MAQVQLVESGGGVVQPGRSLRLSCAASGFTFSSYAMHWVRQAPGKGLEWVAVI
SYDGSNKYYADSVKGRFTISRDNSKNTLYLQMNSLRAEDTAVYYCARDPWGGWL
GSDAFDIWGQGTLVTVSSggggsggggsggggsTQSVLTQPPSASGTPGQRVTISCSGSSS
NIGSNTVNWYQQLPGTAPKLLIYSNNQRPSGVPDRFSGSKSGTSASLAISGLQSED
EADYYCAAYDGDGGEDVFGGGTKLTVLGAA
anti-CSPG4 scFv “HRB300” polypeptide sequence
SEQ ID NO: 29
MAEVQLLESGGGLVQPGGSLRLSCAASGFTFSSYAMSWVRQAPGKGLEWVSAIS
GSGGSTYYADSVKGRFTISRDNSKNTLYLQMNSLRAEDTAVYYCARRYSSGWSYY
FDYWGQGTLVTVSSggggggggsggggsTQSALTQPASVSGSPGQSITISCTGTSSDVGG
YNYVSWYQQHPGKAPKLMIYEVSNRPSGVSNRFSGSKSGNTASLTISGLQAEDEA
DYYCSSYDTFERISVFGGGTKLTVLGAA
anti-CSPG4 scFv “HRB301” polypeptide sequence
SEQ ID NO: 30
MAQVQLVQSGAEVKKPGASVKVSCKASGYTFTSYGISWVRQAPGQGLEWMGWI
SAYNGNTNYAQKLQGRVTMTTDTSTSTAYMELRSLRSDDTAVYYCARRSYDSSGL
DYWGQGTLVTVSSggggsggggsggggsTQSVLTQPPSASGTPGQRVTISCSGSSSNIGSN
TVNWYQQLPGTAPKLLIYSNNQRPSGVPDRFSGSKSGTSASLAISGLQSEDEADY
YCAAWDRRWRLVFGGGTKLTVLGAA
anti-CSPG4 scFv “HRB302” polypeptide sequence
SEQ ID NO: 31
MAQVQLVQSGAEVKKPGASVKVSCKASGYTFTSYGISWVRQAPGQGLEWMGWI
SAYNGNTNYAQKLQGRVTMTTDTSTSTAYMELRSLRSDDTAVYYCARQVGAPTR
FDYWGQGTLVTVSSggggsggggggggsTQSVLTQPPSASGTPGQRVTISCSGSSSNIGS
NTVNWYQQLPGTAPKLLIYSNNQRPSGVPDRFSGSKSGTSASLAISGLQSEDEAD
YYCAAWDTHAWAPVFGGGTKLTVLGAA
anti-CSPG4 scFv “HRB303” polypeptide sequence
SEQ ID NO: 32
MAQVQLVQSGAEVKKPGSSVKVSCKASGGTFSSYAISWVRQAPGQGLEWMGGII
PIFGTANYAQKFQGRVTITADESTSTAYMELSSLRSEDTAVYYCARSKYNWAYKND
YWGQGTLVTVSSggggsggggsggggsTQSVLTQPPSVSAAPGQKVTISCSGSSSNIGNN
YVSWYQQLPGTAPKLLIYDNNKRPSGIPDRFSGSKSGTSATLGITGLQTGDEADY
YCGTYDPWARTAVFGGGTKLTVLGAA
anti-CSPG4 scFv “225.28S” polypeptide sequence
SEQ ID NO: 32
QVKLQQSGGGLVQPGGSMKLSCVVSGFTFSNYWMNWVRQSPEKGLEWIAEIRL
KSNNFGRYYAESVKGRFTISRDDSKSSAYLQMINLRAEDTGIYYCTSYGNYVGHYF
DHWGQGTTVTVSSggggsggggsggggsDIELTQSPKFMSTSVGDRVSVTCKASQNVDT
NVAWYQQKPGQSPEPLLFSASYRYTGVPDRFTGSGSGTDFTLTISNVQSEDLAEY
FCQQYNSYPLTFGGGTKLEIK
anti-BCAN scFv “HRB294” polypeptide sequence
SEQ ID NO: 34
MAQVQLQQSGPGLVKPSQTLSLTCAISGDSVSSNSAAWNWIRQSPSRGLEWLGRT
YYRSKWYNDYAVSVKSRITINPDTSKNQFSLQLNSVTPEDTAVYYCARDQRNYDE
WSGYYPPAELGYYGMDVWGQGTLVTVSSggggsggggsggggsTQSVLTQPPSVSAAPGQ
KVTISCSGSSSNIGNNYVSWYQQLPGTAPKLLIYDNNKRPSGIPDRFSGSKSGTSAT
LGITGLQTGDEADYYCGTWDWSALVVFGGGTKLTVLGAA
anti-BCAN scFv “HRB295” polypeptide sequence
SEQ ID NO: 35
MAQVQLVESGGGVVQPGRSLRLSCAASGFTFSSYAMHWVRQAPGKGLEWVAVI
SYDGSNKYYADSVKGRFTISRDNSKNTLYLQMNSLRAEDTAVYYCARVSDWNDA
AFDIWGQGTLVTVSSggggsggggggggsTQSVLTQPPSASGTPGQRVTISCSGSSSNIG
SNTVNWYQQLPGTAPKLLIYSNNQRPSGVPDRFSGSKSGTSASLAISGLQSEDEAD
YYCAAWDPETARWVFGGGTKLTVLGAA
anti-BCAN scFv “HRB296” polypeptide sequence
SEQ ID NO: 36
MAQVQLQQSGPGLVKPSQTLSLTCAISGDSVSSNSAAWNWIRQSPSRGLEWLGRT
YYRSKWYNDYAVSVKSRITINPDTSKNQFSLQLNSVTPEDTAVYYCARRGEHYDS
SGYYYGLDYWGQGTLVTVSSggggsggggsggggsTQSVLTQPPSVSAAPGQKVTISCSGS
SSNIGNNYVSWYQQLPGTAPKLLIYDNNKRPSGIPDRFSGSKSGTSATLGITGLQT
GDEADYYCGTYDVAAGYVFGGGTKLTVLGAA
anti-BCAN scFv “HRB297” polypeptide sequence
SEQ ID NO: 37
MAQVQLVQSGAEVKKPGSSVKVSCKASGGTFSSYAISWVRQAPGQGLEWMGGII
PIFGTANYAQKFQGRVTITADESTSTAYMELSSLRSEDTAVYYCARPRTAGWSYDA
FDAWGQGTLVTVSSggggsggggsggggsTQSALTQPASVSGSPGQSITISCTGTSSDVGG
YNYVSWYQQHPGKAPKLMIYEVSNRPSGVSNRFSGSKSGNTASLTISGLQAEDEA
DYYCSSYDWRSSGSVFGGGTKLTVLGAA
anti-TNC scFv “E10” polypeptide sequence
SEQ ID NO: 38
EVQLVESGGGLVQPGGSLRLSCAASGFTFSGSRMGWVRQAPGKGLEWVSAINEE
GGQTYYADSVKGRFTISRDNSKNTLYLQMNSLRAEDTAVYYCAKHPPHRPFDYW
GQGTLVTVSRggggsggggsggggsSSELTQDPAVSVALGQTVRITCQGDSLRSYYASW
YQQKPGQAPVLVIYGKNNRPSGIPDRFSGSSSGNTASLTITGAQAEDEADYYCNSS
HGPRRPVVFGGGTKLTVLG
anti-TNC scFv “P12” polypeptide sequence
SEQ ID NO: 39
EVQLVESGGGLVQPGGSLRLSCAASGFTFGQYSMSWVRQAPGKGLEWVSAITGT
GGETYYADSVEGRFTISRDNSKNTLYLQMNSLRAEDTAVYYCAKGRRIFDYWGQ
GTLVTVSRggggsggggsggggsSSELTQDPAVSVALGQTVRITCQGDSLRRQPASWYQ
QKPGQAPVLVIYYKKLRPSGIPDRFSGSSSGNTASLTITGAQAEDEADYYCNSFSP
KPKPVVFGGGTKLTVLG
anti-TNC scFv “F16” polypeptide sequence
SEQ ID NO: 40
EVQLLESGGGLVQPGGSLRLSCAASGFTFSRYGASWVRQAPGKGLEWVSAISGSG
GSTYYADSVKGRFTISRDNSKNTLYLQMNSLRAEDTAVYYCAKAHNAFDYWGQG
TLVTVSRggggsggggsggggsSSELTQDPAVSVALGQTVRITCQGDSLRSYYASWYQQ
KPGQAPVLVIYGKNNRPSGIPDRFSGSSSGNTASLTITGAQAEDEADYYCNSSVYT
MPPVVFGGGTKLTVLG
anti-PTPRZ1 scFv “RRB469” VH polypeptide sequence
SEQ ID NO: 41
MAQVQLVESGGGVVQPGRSLRLSCAASGFTFSSYAMHWVRQAPGKGLEWVAVIS
YDGSNKYYADSVKGRFTISRDNSKNTLYLQMNSLRAEDTAVYYCARGSGYSYGP
GYDAFDIWGQGTLVTVSS
anti-PTPRZ1 scFv “RRB469” VL polypeptide sequence
SEQ ID NO: 42
TNFMLTQPHSVSESPGKTVTISCTRSSGSIASNYVQWYQQRPGSSPTTVIYEDNQRP
SGVPDRFSGSIDSSSNSASLTISGLKTEDEADYYCQSWDPVFGVFGGGTKLTVLGA
A
anti-PTPRZ1 scFv “RRB470” VH polypeptide sequence
SEQ ID NO: 43
MAQVQLVQSGAEVKKPGSSVKVSCKASGGTFSSYAISWVRQAPGQGLEWMGGII
PIFGTANYAQKFQGRVTITADESTSTAYMELSSLRSEDTAVYYCAREGGAVGYYY
GMDVWGQGTLVTVSS
anti-PTPRZ1 scFv “RRB470” VL polypeptide sequence
SEQ ID NO: 44
TQSALTQPASVSGSPGQSITISCTGTSSDVGGYNYVSWYQQHPGKAPKLMIYEVSN
RPSGVSNRFSGSKSGNTASLTISGLQAEDEADYYCSSYDRSNRSMVFGGGTKLTVL
GAA
anti-PTPRZ1 scFv “RRB471” VH polypeptide sequence
SEQ ID NO: 45
MAEVQLVESGGGLVQPGGSLRLSCAASGFTFSSYSMNWVRQAPGKGLEWVSYIS
SSSSTIYYADSVKGRFTISRDNAKNSLYLQMNSLRAEDTAVYYCARPGYGDFPGA
FDIWGQGTLVTVSS
anti-PTPRZ1 scFv “RRB471” VL polypeptide sequence
SEQ ID NO: 46
TQSALTQPASVSGSPGQSITISCTGTSSDVGGYNYVSWYQQHPGKAPKLMIYEVSN
RPSGVSNRFSGSKSGNTASLTISGLQAEDEADYYCSSYDWATYGSVFGGGTKLTV
LGAA
anti-PTPRZ1 scFv “RRB473” VH polypeptide sequence
SEQ ID NO: 47
MAQVQLVESGGGVVQPGRSLRLSCAASGFTFSSYAMHWVRQAPGKGLEWVAVIS
YDGSNKYYADSVKGRFTISRDNSKNTLYLQMNSLRAEDTAVYYCARDQDDSSDA
FDIWGQGTLVTVSS
anti-PTPRZ1 scFv “RRB473” VL polypeptide sequence
SEQ ID NO: 48
TQSVLTQPPSVSAAPGQKVTISCSGSSSNIGNNYVSWYQQLPGTAPKLLIYDNNKR
PSGIPDRFSGSKSGTSATLGITGLQTGDEADYYCGTYDYIATRAVFGGGTKLTVLG
AA
anti-PTPRZ1 scFv “RRB474” VH polypeptide sequence
SEQ ID NO: 49
MAQVQLVQSGAEVKKPGSSVKVSCKASGGTFSSYAISWVRQAPGQGLEWMGGII
PIFGTANYAQKFQGRVTITADESTSTAYMELSSLRSEDTAVYYCARGTYYDFWSG
YYDAFDIWGQGTLVTVSS
anti-PTPRZ1 scFv “RRB474” VL polypeptide sequence
SEQ ID NO: 50
TQSVLTQPPSVSAAPGQKVTISCSGSSSNIGNNYVSWYQQLPGTAPKLLIYDNNKR
PSGIPDRFSGSKSGTSATLGITGLQTGDEADYYCGTWDSSYWQPVFGGGTKLTVL
GAA
anti-PTPRZ1 scFv “RRB476” VH polypeptide sequence
SEQ ID NO: 51
MAQVQLVESGGGVVQPGRSLRLSCAASGFTFSSYAMHWVRQAPGKGLEWVAVIS
YDGSNKYYADSVKGRFTISRDNSKNTLYLQMNSLRAEDTAVYYCARDQDDSSDA
FDIWGQGTLVTVSS
anti-PTPRZ1 scFv “RRB476” VL polypeptide sequence
SEQ ID NO: 52
TQSVLTQPPSVSAAPGQKVTISCSGSSSNIGNNYVSWYQQLPGTAPKLLIYDNNKR
PSGIPDRFSGSKSGTSATLGITGLQTGDEADYYCGTWDYKVSRLVFGGGTKLTVLG
AA
anti-CSPG4 scFv “HRB298” VH polypeptide sequence
SEQ ID NO: 53
MAQVQLVQSGAEVKKPGASVKVSCKASGYTFTSYGISWVRQAPGQGLEWMGWI
SAYNGNTNYAQKLQGRVTMTTDTSTSTAYMELRSLRSDDTAVYYCARRDYYDG
SGFDYWGQGTLVTVSS
anti-CSPG4 scFv “HRB298” VL polypeptide sequence
SEQ ID NO: 54
TQSVLTQPPSVSAAPGQKVTISCSGSSSNIGNNYVSWYQQLPGTAPKLLIYDNNKR
PSGIPDRFSGSKSGTSATLGITGLQTGDEADYYCGTYDGEGRHEVFGGGTKLTVLG
AA
anti-CSPG4 scFv “HRB299” VH polypeptide sequence
SEQ ID NO: 55
MAQVQLVESGGGVVQPGRSLRLSCAASGFTFSSYAMHWVRQAPGKGLEWVAVIS
YDGSNKYYADSVKGRFTISRDNSKNTLYLQMNSLRAEDTAVYYCARDPWGGWL
GSDAFDIWGQGTLVTVSS
anti-CSPG4 scFv “HRB299” VL polypeptide sequence
SEQ ID NO: 56
TQSVLTQPPSASGTPGQRVTISCSGSSSNIGSNTVNWYQQLPGTAPKLLIYSNNQRP
SGVPDRFSGSKSGTSASLAISGLQSEDEADYYCAAYDGDGGEDVFGGGTKLTVLG
AA
anti-CSPG4 scFv “HRB300” VH polypeptide sequence
SEQ ID NO: 57
MAEVQLLESGGGLVQPGGSLRLSCAASGFTFSSYAMSWVRQAPGKGLEWVSAIS
GSGGSTYYADSVKGRFTISRDNSKNTLYLQMNSLRAEDTAVYYCARRYSSGWSY
YFDYWGQGTLVTVSS
anti-CSPG4 scFv “HRB300” VL polypeptide sequence
SEQ ID NO: 58
TQSALTQPASVSGSPGQSITISCTGTSSDVGGYNYVSWYQQHPGKAPKLMIYEVSN
RPSGVSNRFSGSKSGNTASLTISGLQAEDEADYYCSSYDTFERISVFGGGTKLTVLG
AA
anti-CSPG4 scFv “HRB301” VH polypeptide sequence
SEQ ID NO: 59
MAQVQLVQSGAEVKKPGASVKVSCKASGYTFTSYGISWVRQAPGQGLEWMGWI
SAYNGNTNYAQKLQGRVTMTTDTSTSTAYMELRSLRSDDTAVYYCARRSYDSSG
LDYWGQGTLVTVSS
anti-CSPG4 scFv “HRB301” VL polypeptide sequence
SEQ ID NO: 60
TQSVLTQPPSASGTPGQRVTISCSGSSSNIGSNTVNWYQQLPGTAPKLLIYSNNQRP
SGVPDRFSGSKSGTSASLAISGLQSEDEADYYCAAWDRRWRLVFGGGTKLTVLGA
A
anti-CSPG4 scFv “HRB302” VH polypeptide sequence
SEQ ID NO: 61
MAQVQLVQSGAEVKKPGASVKVSCKASGYTFTSYGISWVRQAPGQGLEWMGWI
SAYNGNTNYAQKLQGRVTMTTDTSTSTAYMELRSLRSDDTAVYYCARQVGAPTR
FDYWGQGTLVTVSS
anti-CSPG4 scFv “HRB302” VL polypeptide sequence
SEQ ID NO: 62
TQSVLTQPPSASGTPGQRVTISCSGSSSNIGSNTVNWYQQLPGTAPKLLIYSNNQRP
SGVPDRFSGSKSGTSASLAISGLQSEDEADYYCAAWDTHAWAPVFGGGTKLTVLG
AA
anti-CSPG4 scFv “HRB303” VH polypeptide sequence
SEQ ID NO: 63
MAQVQLVQSGAEVKKPGSSVKVSCKASGGTFSSY AISWVRQAPGQGLEWMGGII
PIFGTANYAQKFQGRVTITADESTSTAYMELSSLRSEDTAVYYCARSKYNWAYKN
DYWGQGTLVTVSS
anti-CSPG4 scFv “HRB303” VL polypeptide sequence
SEQ ID NO: 64
TQSVLTQPPSVSAAPGQKVTISCSGSSSNIGNNYVSWYQQLPGTAPKLLIYDNNKR
PSGIPDRFSGSKSGTSATLGITGLQTGDEADYYCGTYDPWARTAVFGGGTKLTVLG
AA
anti-CSPG4 scFv “225.28S” VH polypeptide sequence
SEQ ID NO: 65
QVKLQQSGGGLVQPGGSMKLSCVVSGFTFSNYWMNWVRQSPEKGLEWIAEIRLK
SNNFGRYYAESVKGRFTISRDDSKSSAYLQMINLRAEDTGIYYCTSYGNYVGHYF
DHWGQGTTVTVSS
anti-CSPG4 scFv “225.28S” VL polypeptide sequence
SEQ ID NO: 66
DIELTQSPKFMSTSVGDRVSVTCKASQNVDTNVAWYQQKPGQSPEPLLFSASYRY
TGVPDRFTGSGSGTDFTLTISNVQSEDLAEYFCQQYNSYPLTFGGGTKLEIK
anti-BCAN scFv “HRB294” VH polypeptide sequence
SEQ ID NO: 67
MAQVQLQQSGPGLVKPSQTLSLTCAISGDSVSSNSAAWNWIRQSPSRGLEWLGRT
YYRSKWYNDYAVSVKSRITINPDTSKNQFSLQLNSVTPEDTAVYYCARDQRNYDF
WSGYYPPAELGYYGMDVWGQGTLVTVSS
anti-BCAN scFv “HRB294” VL polypeptide sequence
SEQ ID NO: 68
TQSVLTQPPSVSAAPGQKVTISCSGSSSNIGNNYVSWYQQLPGTAPKLLIYDNNKR
PSGIPDRFSGSKSGTSATLGITGLQTGDEADYYCGTWDWSALVVFGGGTKLTVLG
AA
anti-BCAN scFv “HRB295” VH polypeptide sequence
SEQ ID NO: 69
MAQVQLVESGGGVVQPGRSLRLSCAASGFTFSSYAMHWVRQAPGKGLEWVAVIS
YDGSNKYYADSVKGRFTISRDNSKNTLYLQMNSLRAEDTAVYYCARVSDWNDA
AFDIWGQGTLVTVSS
anti-BCAN scFv “HRB295” VL polypeptide sequence
SEQ ID NO: 70
TQSVLTQPPSASGTPGQRVTISCSGSSSNIGSNTVNWYQQLPGTAPKLLIYSNNQRP
SGVPDRFSGSKSGTSASLAISGLQSEDEADYYCAAWDPETARWVFGGGTKLTVLG
AA
anti-BCAN scFv “HRB296” VH polypeptide sequence
SEQ ID NO: 71
MAQVQLQQSGPGLVKPSQTLSLTCAISGDSVSSNSAAWNWIRQSPSRGLEWLGRT
YYRSKWYNDYAVSVKSRITINPDTSKNQFSLQLNSVTPEDTAVYYCARRGEHYDS
SGYYYGLDYWGQGTLVTVSS
anti-BCAN scFv “HRB296” VL polypeptide sequence
SEQ ID NO: 72
TQSVLTQPPSVSAAPGQKVTISCSGSSSNIGNNYVSWYQQLPGTAPKLLIYDNNKR
PSGIPDRFSGSKSGTSATLGITGLQTGDEADYYCGTYDVAAGYVFGGGTKLTVLG
AA
anti-BCAN scFv “HRB297” VH polypeptide sequence
SEQ ID NO: 73
MAQVQLVQSGAEVKKPGSSVKVSCKASGGTFSSYAISWVRQAPGQGLEWMGGII
PIFGTANYAQKFQGRVTITADESTSTAYMELSSLRSEDTAVYYCARPRTAGWSYD
AFDAWGQGTLVTVSS
anti-BCAN scFv “HRB297” VL polypeptide sequence
SEQ ID NO: 74
TQSALTQPASVSGSPGQSITISCTGTSSDVGGYNYVSWYQQHPGKAPKLMIYEVSN
RPSGVSNRFSGSKSGNTASLTISGLQAEDEADYYCSSYDWRSSGSVFGGGTKLTVL
GAA
anti-TNC scFv “E10” VH polypeptide sequence
SEQ ID NO: 75
EVQLVESGGGLVQPGGSLRLSCAASGFTFSGSRMGWVRQAPGKGLEWVSAINEE
GGQTYYADSVKGRFTISRDNSKNTLYLQMNSLRAEDTAVYYCAKHPPHRPFDYW
GQGTLVTVSR
anti-TNC scFv “E10” VL polypeptide sequence
SEQ ID NO: 76
SSELTQDPAVSVALGQTVRITCQGDSLRSYYASWYQQKPGQAPVLVIYGKNNRPS
GIPDRFSGSSSGNTASLTITGAQAEDEADYYCNSSHGPRRPVVFGGGTKLTVLG
anti-TNC scFv “P12” VH polypeptide sequence
SEQ ID NO: 77
EVQLVESGGGLVQPGGSLRLSCAASGFTFGQYSMSWVRQAPGKGLEWVSAITGT
GGETYYADSVEGRFTISRDNSKNTLYLQMNSLRAEDTAVYYCAKGRRIFDYWGQ
GTLVTVSR
anti-TNC scFv “P12” VL polypeptide sequence
SEQ ID NO: 78
SSELTQDPAVSVALGQTVRITCQGDSLRRQPASWYQQKPGQAPVLVIYYKKLRPS
GIPDRFSGSSSGNTASLTITGAQAEDEADYYCNSFSPKPKPVVFGGGTKLTVLG
anti-TNC scFv “F16” VH polypeptide sequence
SEQ ID NO: 79
EVQLLESGGGLVQPGGSLRLSCAASGFTFSRYGASWVRQAPGKGLEWVSAISGSG
GSTYYADSVKGRFTISRDNSKNTLYLQMNSLRAEDTAVYYCAKAHNAFDYWGQ
GTLVTVSR
anti-TNC scFv “F16” VL polypeptide sequence
SEQ ID NO: 80
SSELTQDPAVSVALGQTVRITCQGDSLRSYYASWYQQKPGQAPVLVIYGKNNRPS
GIPDRFSGSSSGNTASLTITGAQAEDEADYYCNSSVYTMPPVVFGGGTKLTVLG

CDRs of SEQ ID NOs: 83-93 are annotated as follows: CDR1 (underlined), CDR2 (bold and underlined) and CDR3 (italicised and underlined). CDRs were predicted with the Benchling's Antibody Property Prediction Tools, where annotations were assigned using the North CDR definition of the Sequence-based antibody CAnonical LOOP (SCALOP) structure annotation developed by the Oxford Protein Informatics Group (Dunbar et al, SAbPred: a structure-based antibody prediction server, Nucleic Acids Research, Volume 44, Issue W1, 8 Jul. 2016, Pages W474-W478, https://doi.org/10.1093/nar/gkw361)

Anti-BCAN “RB826” VHH polypeptide sequence
SEQ ID NO: 83
EVQLQESGGGFVQAGGSLRLSCAASGRLRPFERMGWFRQAPGKEREFVAAISVH
DEVFPYYADSVKGRFTISRDNAKNTVYLQMNSLKPEDTATYYCAFFIMDDVKYW
GQGTQVTVSS
Anti-BCAN “RB827” VHH polypeptide sequence
SEQ ID NO: 84
EVQLQESGGGFVQAGGSLRLSCAASGESFVPEMGWFRQAPGKEREFVAAISVEQS
LDMYYADSVKGRFTISRDNAKNTVYLQMNSLKPEDTATYYCAIVAIDDENTYWGQ
GTQVTVSS
Anti-BCAN “RB828” VHH polypeptide sequence
SEQ ID NO: 85
EVQLQESGGGFVQAGGSLRLSCAASGVSLRTRSMGWFRQAPGKEREFVAAISRH
NDHEFYYADSVKGRFTISRDNAKNTVYLQMNSLKPEDTATYYCAKLPVNPNLHY
WGQGTQVTVSS
Anti-BCAN “RB829” VHH polypeptide sequence
SEQ ID NO: 86
EVQLQESGGGFVQAGGSLRLSCAASGVHVPLQNMGWFRQAPGKEREFVAAISRD
MPVDNYYADSVKGRFTISRDNAKNTVYLQMNSLKPEDTATYYCAVRVYTTSLWY
WGQGTQVTVSS
Anti-CSPG4 “RB830” VHH polypeptide sequence
SEQ ID NO: 87
EVQLQESGGGFVQAGGSLRLSCAASGNVQRRFRMGWFRQAPGKEREFVAAISTN
RDRRNYYADSVKGRFTISRDNAKNTVYLQMNSLKPEDTATYYCAVMNKNFTYMY
WGQGTQVTVSS
Anti-CSPG4 “RB831” VHH polypeptide sequence
SEQ ID NO: 88
EVQLQESGGGFVQAGGSLRLSCAASGEPVHSTSMGWFRQAPGKEREFVAAISLNV
MHSRYYADSVKGRFTISRDNAKNTVYLQMNSLKPEDTATYYCASYPHYMTPMYW
GQGTQVTVSS
Anti-PTPRZ1 “RB832” VHH polypeptide sequence
SEQ ID NO: 89
EVQLQESGGGFVQAGGSLRLSCAASGSDVTRLNMGWFRQAPGKEREFVAAISRS
EQNRLYYADSVKGRFTISRDNAKNTVYLQMNSLKPEDTATYYCATTALAAVTKAT
HYWGQGTQVTVSS
Anti-PTPRZ1 “RB833” VHH polypeptide sequence
SEQ ID NO: 90
EVQLQESGGGFVQAGGSLRLSCAASGNVVFLTSMGWFRQAPGKEREFVAAISRSF
FDDPYYADSVKGRFTISRDNAKNTVYLQMNSLKPEDTATYYCAPYKRDYRQHTVP
RHIYWGQGTQVTVSS
Anti-PTPRZ1 “RB834” VHH polypeptide sequence
SEQ ID NO: 91
EVQLQESGGGFVQAGGSLRLSCAASGSSSRLFNMGWFRQAPGKEREFVAAISHM
ENDVDYYADSVKGRFTISRDNAKNTVYLQMNSLKPEDTATYYCALMLKGWDHST
RYYWGQGTQVTVSS
Anti-TNC “RB835” VHH polypeptide sequence
SEQ ID NO: 92
EVQLQESGGGFVQAGGSLRLSCAASGNLMVRREMGWFRQAPGKEREFVAAISRS
SQEEVYYADSVKGRFTISRDNAKNTVYLQMNSLKPEDTATYYCAMEGFYVYNQY
WGQGTQVTVSS
Anti-TNC “RB836” VHH polypeptide sequence
SEQ ID NO: 93
EVQLQESGGGFVQAGGSLRLSCAASGRRVTVSEMGWFRQAPGKEREFVAAISMR
ERESMYYADSVKGRFTISRDNAKNTVYLQMNSLKPEDTATYYCAEYTHWYSHPY
WGQGTQVTVSS
Anti-BCAN “RB826” VHH polynucleotide sequence
SEQ ID NO: 94
GAGGTTCAACTGCAGGAGAGCGGCGGTGGATTTGTGCAAGCAGGGGGGTCCTT
GCGATTATCATGTGCTGCATCGGGGCGGCTGAGACCATTCGAACGTATGGGCT
GGTTCCGCCAGGCACCTGGTAAAGAGAGGGAGTTTGTGGCCGCCATAAGCGTG
CACGATGAAGTGTTTCCGTACTACGCCGATTCCGTCAAGGGCAGGTTTACCATT
AGTAGAGACAATGCGAAAAACACAGTCTATCTTCAGATGAACTCTCTCAAGCC
CGAAGATACTGCTACATATTACTGCGCTTTCTTCATCATGGACGACGTTAAGTA
TTGGGGACAGGGAACCCAGGTAACGGTGTCATCT
Anti-BCAN “RB827” VHH polynucleotide sequence
SEQ ID NO: 95
GAGGTGCAATTGCAGGAGAGCGGCGGTGGATTTGTGCAGGCCGGAGGGTCCCT
CCGACTTTCATGCGCTGCATCTGGGGAAAGCTTCGTTCCAGAAATGGGCTGGTT
CCGCCAGGCACCTGGTAAAGAGCGGGAGTTTGTGGCCGCCATAAGCGTAGAAC
AGAGTCTGGATATGTATTACGCCGATTCCGTCAAGGGCAGGTTTACCATTAGT
AGAGACAATGCGAAAAACACTGTCTATCTGCAGATGAATTCTTTAAAGCCCGA
GGATACTGCTACATACTACTGTGCTATCGTTGCTATCGACGACTTCAACACATA
TTGGGGACAGGGGACCCAAGTGACGGTGTCATCC
Anti-BCAN “RB828” VHH polynucleotide sequence
SEQ ID NO: 96
GAGGTGCAATTGCAGGAGAGTGGCGGGGGATTTGTGCAAGCAGGAGGGTCCC
TCCGACTTTCATGCGCTGCATCTGGTGTGAGCCTGAGAACCCGGAGCATGGGC
TGGTTCCGCCAGGCACCAGGTAAAGAGAGGGAATTTGTTGCCGCCATCAGCCG
TCACAACGATCATGAATTCTATTACGCCGATTCCGTCAAGGGCAGGTTCACCAT
TAGTAGAGACAATGCTAAGAACACAGTCTATTTACAGATGAATTCCCTCAAGC
CCGAAGACACTGCTACGTACTACTGTGCTAAACTGCCTGTTAACCCGAATCTGC
ACTATTGGGGACAGGGGACACAGGTAACTGTGTCATCT
Anti-BCAN “RB829” VHH polynucleotide sequence
SEQ ID NO: 97
GAGGTGCAATTACAGGAAAGCGGCGGTGGATTTGTCCAGGCTGGGGGGTCCCT
CCGACTGTCATGTGCTGCAAGCGGTGTGCACGTCCCTCTGCAGAACATGGGCT
GGTTCCGCCAAGCACCGGGGAAAGAGAGGGAGTTCGTTGCCGCCATTAGCCGG
GATATGCCAGTCGATAACTATTACGCCGACTCCGTGAAGGGCAGGTTTACCAT
CAGTAGAGACAATGCGAAAAACACGGTGTACCTTCAGATGAATAGTTTGAAGC
CCGAAGACACTGCTACATATTACTGCGCTGTTCGTGTGTACACTACCTCTCTGT
GGTATTGGGGACAGGGAACCCAGGTAACAGTGTCATCT
Anti-CSPG4 “RB830” VHH polynucleotide sequence
SEQ ID NO: 98
GAGGTGCAGCTGCAGGAGAGCGGCGGGGGATTTGTCCAAGCTGGTGGGTCCTT
GCGACTGTCATGCGCTGCATCGGGTAACGTTCAGAGACGGTTCAGAATGGGCT
GGTTCCGCCAGGCACCTGGGAAAGAGAGGGAATTTGTTGCCGCCATTAGCACA
AACCGGGATAGGCGCAACTATTACGCCGACTCCGTCAAGGGCAGGTTTACCAT
CAGTCGTGACAATGCGAAAAACACGGTGTACCTTCAGATGAATTCTCTCAAGC
CCGAAGATACTGCTACATATTACTGTGCTGTGATGAATAAGAATTTCACTTACA
TGTATTGGGGACAGGGAACCCAAGTAACCGTGTCATCT
Anti-CSPG4 “RB831” VHH polynucleotide sequence
SEQ ID NO: 99
GAGGTACAGCTTCAAGAAAGCGGCGGGGGATTCGTCCAGGCGGGTGGGTCCTT
GCGGCTGTCATGCGCTGCATCTGGGGAACCAGTGCACAGTACAAGTATGGGCT
GGTTCCGCCAAGCACCGGGTAAAGAGAGGGAGTTTGTTGCCGCCATCAGCCTG
AACGTTATGCACAGCAGATATTACGCCGATTCCGTCAAGGGCCGATTTACCAT
TTCGCGTGACAATGCAAAAAACACCGTGTATTTACAGATGAATTCCCTCAAGC
CCGAAGACACTGCTACGTATTACTGTGCTTCTTACCCTCATTACATGACTCCCA
TGTATTGGGGACAGGGAACCCAGGTGACAGTGTCATCT
Anti-PTPRZ1 “RB832” VHH polynucleotide sequence
SEQ ID NO: 100
GAGGTGCAATTACAGGAAAGCGGCGGTGGATTTGTCCAGGCAGGTGGGTCCCT
CCGACTCTCATGCGCCGCATCTGGGAGCGACGTCACACGTCTGAACATGGGCT
GGTTCCGCCAGGCACCTGGGAAGGAGAGGGAGTTTGTTGCCGCCATTAGCAGA
AGTGAACAGAACCGGCTGTACTACGCCGATTCCGTGAAGGGCAGGTTCACCAT
CAGTAGAGACAATGCGAAAAACACCGTTTATCTTCAGATGAATTCTTTGAAGC
CCGAAGATACTGCTACCTATTACTGTGCAACAACAGCTCTGGCTGCTGTGACTA
AAGCCACTCACTATTGGGGACAGGGAACCCAAGTAACGGTGTCATCC
Anti-PTPRZ1 “RB833” VHH polynucleotide sequence
SEQ ID NO: 101
GAGGTACAGCTGCAAGAGAGCGGCGGGGGATTTGTCCAGGCTGGTGGGTCCCT
CCGACTTTCATGTGCTGCATCTGGTAACGTTGTGTTCCTGACGTCGATGGGCTG
GTTCCGCCAGGCACCCGGGAAGGAGAGGGAATTTGTTGCCGCCATAAGCCGGA
GCTTCTTCGATGATCCATATTACGCCGATTCCGTCAAGGGCAGGTTTACCATTA
GTAGAGACAATGCAAAAAACACAGTGTATTTACAGATGAATAGTTTGAAGCCC
GAAGACACTGCTACATATTACTGCGCTCCGTACAAAAGAGACTACCGGCAGCA
CACTGTGCCTCGTCATATCTATTGGGGACAGGGAACCCAAGTGACCGTGTCAT
CT
Anti-PTPRZ1 “RB834” VHH polynucleotide sequence
SEQ ID NO: 102
GAGGTGCAGCTTCAAGAGAGCGGCGGGGGATTTGTCCAAGCTGGTGGGTCCCT
CCGATTATCATGCGCTGCATCTGGGAGCAGTAGTAGACTGTTCAACATGGGCT
GGTTCCGCCAGGCACCTGGTAAAGAGCGGGAATTTGTGGCCGCCATTAGCCAC
ATGGAAAACGATGTTGATTATTACGCCGATTCCGTGAAGGGCAGGTTCACCAT
CTCCAGAGACAATGCGAAGAACACAGTCTATTTGCAGATGAATTCTCTCAAGC
CCGAAGACACTGCTACTTATTACTGTGCTCTGATGCTGAAAGGCTGGGACCATT
CGACACGTTACTACTGGGGACAGGGAACCCAGGTAACGGTGTCATCT
Anti-TNC “RB835” VHH polynucleotide sequence
SEQ ID NO: 103
GAGGTACAGCTTCAGGAGAGCGGCGGCGGATTTGTGCAAGCAGGTGGGTCCCT
CCGATTATCATGCGCTGCATCCGGTAACCTGATGGTCCGTCGGGAAATGGGCT
GGTTCCGCCAGGCACCTGGGAAAGAGAGGGAGTTCGTTGCCGCCATTAGCAGA
AGCAGTCAGGAGGAAGTTTATTACGCCGACTCCGTCAAGGGCAGGTTTACCAT
CAGTAGAGACAATGCGAAAAACACAGTGTATCTGCAGATGAATTCTTTGAAGC
CCGAAGATACTGCTACGTATTACTGTGCTATGGAAGGGTTCTACGTGTACAAC
CAGTATTGGGGACAGGGAACCCAAGTGACAGTGTCATCT
Anti-TNC “RB836” VHH polynucleotide sequence
SEQ ID NO: 104
GAGGTGCAACTGCAGGAGAGCGGCGGGGGATTTGTGCAGGCAGGGGGGTCCC
TCCGACTTTCATGCGCTGCATCCGGTAGAAGAGTAACGGTCAGTGAAATGGGC
TGGTTCCGCCAGGCACCTGGTAAAGAGAGGGAGTTTGTTGCCGCCATCAGCAT
GCGGGAACGGGAAAGCATGTATTACGCCGACTCCGTGAAGGGCAGGTTCACC
ATTAGTCGTGACAATGCTAAAAACACCGTCTATCTGCAGATGAACTCGTTGAA
GCCCGAAGATACTGCTACATATTACTGTGCTGAGTACACTCACTGGTACTCTCA
TCCATATTGGGGACAGGGAACCCAAGTTACAGTGTCATCT

Hinge sequences, transmembrane domain sequences and intracellular domain sequences are provided below.

human CD8a hinge polypeptide sequence
SEQ ID NO: 117
TTTPAPRPPTPAPTIASQPLSLRPEACRPAAGGAVHTRGLDFACD
human IgG4 hinge polypeptide sequence
SEQ ID NO: 118
ESKYGPPCPPCP
human IgG1 hinge polypeptide sequence
SEQ ID NO: 119
EPKSPDKTHTCP
human IgG1 hinge polypeptide sequence
SEQ ID NO: 120
EPKSCDKTHTCP
human IgGI long hinge polypeptide sequence
SEQ ID NO: 121
AEPKSPDKTHTCPPCPAPPVAGPSVFLFPPKPKDTLMIARTPEVTCVVVDVSHEDPE
VKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHY
TQKSLSLSPGKKDPK
human CD8a transmembrane domain polypeptide sequence
SEQ ID NO: 122
IYIWAPLAGTCGVLLLSLVITLYC
human CD28 transmembrane domain polypeptide sequence
SEQ ID NO: 123
FWVLVVVGGVLACYSLLVTVAFIIFWV
human 4-1BB intracellular domain polypeptide sequence
SEQ ID NO: 124
KRGRKKLLYIFKQPFMRPVQTTQEEDGCCRFPEEEEGGCEL
human CD28 intracellular domain polypeptide sequence
SEQ ID NO: 125
RSKRSRGGHSDYMNMTPRRPGPTRKHYQPYAPPRDFAAYRS
human CD3z intracellular domain polypeptide sequence
no. 1
SEQ ID NO: 126
RVKFSRSADAPAYKQGQNQLYNELNLGRREEYDVLDKRRGRDPEMGGKPRRKN
PQEGLYNELQKDKMAEAYSEIGMKGERRRGKGHDGLYQGLSTATKDTYDALHM
QALPPR
human CD3z intracellular domain polypeptide sequence
no. 2
SEQ ID NO: 127
RVKFSRSADAPAYQQGQNQLYNELNLGRREEYDVLDKRRGRDPEMGGKPQRRK
NPQEGLYNELQKDKMAEAYSEIGMKGERRRGKGHDGLYQGLSTATKDTYDALH
MQALPPR
human CD3z intracellular domain polypeptide sequence
no. 3
SEQ ID NO: 128
RVKFSRSADAPAYQQGQNQLYNELNLGRREEYDVLDKRRGRDPEMGGKPQRRK
NPQEGLYNELQKDKMAEAYSEIGMKGERRRGKGHDGLYQGLSTATKDTYDALH
MQARRA
human CD8a hinge polynucleotide sequence
SEQ ID NO: 129
ACCACGACGCCAGCGCCGCGACCACCAACACCGGCGCCCACCATCGCGTCGCA
GCCCCTGTCCCTGCGCCCAGAGGCGTGCCGGCCAGCGGGGGGGGCGCAGTGC
ACACGAGGGGGCTGGACTTCGCCTGTGAT
human IgG4 hinge polynucleotide sequence
SEQ ID NO: 130
GAGTCAAAGTATGGGCCTCCATGTCCTCCATGTCCG
human IgG1 hinge polynucleotide sequence
SEQ ID NO: 131
GAGCCGAAGTCGCCAGACAAAACTCACACTTGTCCT
human IgG1 long hinge polynucleotide sequence
SEQ ID NO: 132
GCAGAGCCGAAGTCGCCAGACAAAACTCACACTTGTCCTCCTTGTCCAGCCCC
CCCCGTGGCGGGTCCCAGCGTGTTCCTGTTTCCTCCGAAGCCAAAAGATACCCT
GATGATCGCACGCACCCCCGAAGTAACGTGCGTGGTGGTCGATGTGTCACATG
AGGACCCTGAGGTCAAATTCAATTGGTACGTTGACGGGGTAGAAGTTCACAAC
GCTAAAACCAAGCCAAGGGAGGAGCAGTACAACAGCACCTATCGAGTGGTGA
GTGTACTGACCGTCCTACACCAAGATTGGTTGAATGGCAAGGAATACAAGTGT
AAGGTGTCCAACAAGGCTTTACCTGCTCCTATCGAGAAGACAATTTCTAAGGC
CAAAGGCCAGCCCAGAGAGCCACAGGTTTACACACTCCCACCATCACGTGACG
AGCTTACGAAAAATCAGGTCAGTCTGACTTGCCTCGTTAAAGGATTTTACCCTA
GTGACATAGCCGTGGAATGGGAAAGCAACGGCCAGCCCGAGAATAATTATAA
AACAACACCGCCCGTGCTCGACTCTGATGGTTCTTTTTTCCTGTATTCCAAACT
GACCGTCGATAAGAGCCGGTGGCAGCAGGGAAACGTGTTCTCCTGCTCCGTCA
TGCATGAAGCCTTGCATAACCACTATACTCAAAAGTCACTGTCTCTTAGCCCTG
GGAAGAAAGATCCCAAG
human CD8a transmembrane domain polynucleotide sequence
SEQ ID NO: 133
ATCTACATCTGGGCGCCCTTGGCCGGGACTTGTGGGGTCCTTCTCCTGTCACTG
GTTATCACCCTTTACTGC
human CD28 transmembrane domain polynucleotide sequence
SEQ ID NO: 134
TTCTGGGTGCTGGTGGTCGTGGGCGGCGTGCTGGCCTGTTACAGCCTGCTCGTG
ACCGTGGCCTTCATCATCTTTTGGGTC
human 4-1BB intracellular domain polynucleotide sequence
SEQ ID NO: 135
AAACGGGGCAGAAAGAAACTCCTGTATATATTCAAACAACCATTTATGAGACC
AGTACAAACTACTCAAGAGGAAGATGGCTGTAGCTGCCGATTTCCAGAAGAAG
AAGAAGGAGGATGTGAACTG
human CD28 intracellular domain polynucleotide sequence
SEQ ID NO: 136
CGAAGCAAGCGGAGCAGAGGCGGCCACAGCGACTACATGAACATGACCCCCA
GACGGCCTGGCCCCACCCGGAAGCACTACCAGCCTTACGCCCCTCCCAGAGAC
TTCGCCGCCTACAGAAGC
human CD3z intracellular domain polynucleotide sequence
no. 1
SEQ ID NO: 137
AGAGTGAAGTTCAGCAGGAGCGCAGACGCCCCCGCGTACAAGCAGGGCCAGA
ACCAGCTCTATAACGAGCTCAATCTAGGACGAAGAGAGGAGTACGATGTTTTG
GACAAGAGACGTGGCCGGGACCCTGAGATGGGGGGAAAGCCGAGAAGGAAG
AACCCTCAGGAAGGCCTGTACAATGAACTGCAGAAAGATAAGATGGCGGAGG
CCTACAGTGAGATTGGGATGAAAGGCGAGCGCCGGAGGGGCAAGGGGCACGA
TGGCCTTTACCAGGGTCTCAGTACAGCCACCAAGGACACCTACGACGCCCTTC
ACATGCAGGCCCTGCCCCCTCGC
human CD3z intracellular domain polynucleotide sequence
no. 2
SEQ ID NO: 138
AGAGTGAAGTTCAGCCGCAGCGCCGACGCCCCTGCCTACCAGCAGGGACAGA
ACCAGCTGTACAACGAGCTGAACCTGGGCAGACGGGAAGAGTACGACGTGCT
GGACAAGCGGAGAGGCCGGGACCCTGAGATGGGCGGCAAGCCCCAGCGGCGG
AAGAACCCCCAGGAAGGCCTGTATAACGAACTGCAGAAAGACAAGATGGCCG
AGGCCTACAGCGAGATCGGCATGAAGGGCGAGCGGCGGAGAGGCAAGGGCCA
CGATGGCCTGTACCAGGGCCTGAGCACCGCCACCAAGGACACCTACGACGCCC
TGCACATGCAGGCCCTGCCCCCTAGG
human CD3z intracellular domain polynucleotide sequence
no. 3
SEQ ID NO: 139
AGAGTGAAGTTCAGCCGCAGCGCCGACGCCCCTGCCTACCAGCAGGGACAGA
ACCAGCTGTACAACGAGCTGAACCTGGGCAGACGGGAAGAGTACGACGTGCT
GGACAAGCGGAGAGGCCGGGACCCTGAGATGGGCGGCAAGCCCCAGCGGCGG
AAGAACCCCCAGGAAGGCCTGTATAACGAACTGCAGAAAGACAAGATGGCCG
AGGCCTACAGCGAGATCGGCATGAAGGGCGAGCGGCGGAGAGGCAAGGGCCA
CGATGGCCTGTACCAGGGCCTGAGCACCGCCACCAAGGACACCTACGACGCCC
TGCACATGCAGGCCCggcgcgcc

### DETAILED DESCRIPTION

The invention relates to an immune effector cell or a population of immune effector cells expressing one or more chimeric antigen receptors (CARs) specific for one or more glioma-associated antigens. The antigens receptor-type tyrosine-protein phosphatase zeta (PTPRZ1), brevican core protein (BCAN), chondroitin sulfate proteoglycan 4 (CSPG4) and tenascin (TNC) have recently been found to be expressed in glioma (Dutoit et al, Brain 135.4 (2012): 1042-1054). A multipeptide vaccine comprising peptides from these antigens has been created (Dutoit et al, Oncoimmunology 7.2 (2018): e1391972.; Migliorini et al, Neuro-oncology 21.7 (2019): 923-933.) and is undergoing clinical trials (NCT03665545, NCT02924038).

The inventors have shown that immune effector cells expressing CARs specific for one or more glioma-associated antigens are able to generate a specific immune response to cells expressing the glioma-associated antigens as measured by cytotoxicity assays and T-cell activation.

Until now, CAR T cell approaches have been investigated clinically for recurrent GBM (targeting EGFRvIII, IL13Ra2, Her2) in the monovalent format, i.e., targeting one antigen at a time. Some patients displayed disease stabilisation but tumours invariably recurred and loss of the epitope targeted by the monovalent CAR-T cell used was seen in some instances. The present inventors have identified new glioma-associated antigens that have been targeted in a monovalent format and further demonstrated their efficacy in a multivalent targeting approach. Bielamowicz et al (Neuro-oncology 20.4 (2018): 506-518) describes a multivalent approach in which a T cell was engineered to express three CAR molecules with distinct antigen specificity. Accordingly, in one aspect, the invention provides an immune effector cell expressing two or more CARs specific for different glioma-associated antigens. In one aspect, the invention provides a population of immune effector cells comprising at least two different CAR-expressing immune effector cells, wherein each different CAR-expressing immune effector cell is specific for a different glioma-associated antigen. Typically, one or more of the glioma-associated antigens is selected from PTPRZ1, BCAN, CSPG4 and TNC. In some cases, two or more of the glioma-associated antigens are selected from PTPRZ1, BCAN, CSPG4 and TNC.

The invention also provides an immune effector cell or a population of immune effector cells expressing a chimeric antigen receptor (CAR) specific for PTPRZ1. Additionally, the invention provides an immune effector cell or a population of immune effector cells expressing a chimeric antigen receptor (CAR) specific for BCAN. The inventors have surprisingly shown for the first time that an immune effector cell expressing a CAR specific for PTPRZ1 or BCAN has therapeutic utility, specifically for the treatment of glioblastoma. Any antigen-binding domain specific for PTPRZ1 or for BCAN may be used, such as those known in the art. Preferably, the antigen-binding domain is an antigen-binding domain disclosed herein.

In some cases, the invention makes use of the ‘bystander’ effect, as described in the Examples of the application. For example, the immune effector cells of the invention comprising one or more CARs specific for a glioma associated antigen may be capable of killing cancer cells comprising a mix of cancer cells that express the glioma-associated antigen and cancer cells that do not express the glioma-associated antigen. The cancer cells not expressing the glioma-associated antigen are believed to be killed via soluble factors, potentially allowing for greater therapeutic efficacy.

Furthermore, lentivirally-transduced CAR T cells are associated with toxicity, including cytokine release syndrome and neurotoxicity. In some aspects, the immune effector cells of the invention are transduced with RNA in order to overcome these issues.

### Chimeric Antigen Receptors

Chimeric antigen receptors (CARs) are expressed in immune effector cells. CARs comprise an extracellular antigen-binding domain. CARs generally comprise an extracellular antigen-binding domain and an intracellular cytoplasmic signalling domain. CARs may comprise an extracellular antigen-binding domain, a transmembrane domain and an intracellular cytoplasmic signalling domain. The CAR may also comprise an extracellular spacer and/or a hinge between the transmembrane domain and the antigen-binding domain and/or between the transmembrane domain and the cytoplasmic signalling domain. Preferably, the CAR comprises a hinge between the transmembrane domain and the antigen-binding domain.

The cytoplasmic signalling domain may comprise an activation domain. The activation domain serves to activate the immune effector cell following engagement of the extracellular domain (e.g. scFv). The cytoplasmic signalling domain may comprise one or more of (i) a CD35 (zeta) activation domain, (ii) a 4-1BB (CD137) activation domain, (iii) a CD3& (epsilon) activation domain, (iv) an OX40 (CD134) activation domain, (v) a CD28 activation domain, and/or (vi) a CD27 activation domain.

The CD3zeta activation is comprised in the signalling domain of first generation CARs. Preferably, the cytoplasmic domain comprises a CD3 (zeta) activation domain.

Second generation CARs comprise a CD3zeta activation domain and a CD28 activation domain. The cytoplasmic domain comprises a CD35 (zeta) activation domain and a CD28 activation domain.

Third generation CARs comprise additional domains such as the 4-1BB activation domain or OX40 (CD134) activation domain. Preferably, the cytoplasmic domain comprises a 4-1BB activation domain and a CD35 (zeta) activation domain.

The cytoplasmic signalling domain may comprise a 4-1BBz domain, which comprises CD3zeta and the 4-1BBz activation domains. The cytoplasmic signalling domain may comprise a CD28z domain, which comprises CD3zeta and the CD28 activation domains. As illustrated in the Examples, a CAR comprising a CD28z domain in particularly useful in the invention, for example, when the immune effector cell is transduced with RNA encoding the CAR. The cytoplasmic signalling domain may comprise a 4-1BBz+CD28z domain, which comprises CD3zeta, CD28 and the 4-1BBz activation domains.

The cytoplasmic signalling domain may comprise the CD35 (zeta) activation domain alone or in combination with a CD28, CD27, OX-40 (CD134) and/or 4-1BB (CD137) domain.

Other activation domains include IL-15Ra, CD2, CDS, ICAM-1, LTA-1 and ICOS and may be used in combination with the activation domains described above.

If the immune effector cell in which the CAR is expressed is a phagocyte, the intracellular signalling domain may comprise the intracellular domain of Megf10 or FcRv.

In some cases, the 4-1BB activation domain comprises or consists of an amino acid sequence having at least 70% identity to SEQ ID NO: 124, such at least 80%, at least 90%, at least 95%, at least 99% or 100% identity to SEQ ID NO: 124. In some cases, the CD28 activation domain comprises or consists of an amino acid sequence having at least 70% identity to SEQ ID NO: 125, such at least 80%, at least 90%, at least 95%, at least 99% or 100% identity to SEQ ID NO: 125. In some cases, the CD3zeta activation domain comprises or consists of an amino acid sequence having at least 70% identity to any one of SEQ ID NOs: 126-128, such at least 80%, at least 90%, at least 95%, at least 99% or 100% identity to any one of SEQ ID NOs: 126-128. For example, the 4-1BBz domain may comprise an amino acid sequence having at least 70% identity to SEQ ID NO: 124 (such at least 80%, at least 90%, at least 95%, at least 99% or 100% identity to SEQ ID NO: 124) and an amino acid sequence having at least 70% identity to any one of SEQ ID NOs: 126-128 (such at least 80%, at least 90%, at least 95%, at least 99% or 100% identity to any one of SEQ ID NOs: 126-128). Similarly, a CD28z domain may comprise an amino acid sequence having at least 70% identity to SEQ ID NO: 125 (such at least 80%, at least 90%, at least 95%, at least 99% or 100% identity to SEQ ID NO: 125) and an amino acid sequence having at least 70% identity to any one of SEQ ID NOs: 126-128 (such at least 80%, at least 90%, at least 95%, at least 99% or 100% identity to any one of SEQ ID NOs: 126-128).

In some cases, the 4-1BB activation domain comprises or consists of an amino acid sequence having at least 70% identity to the amino acid sequence encoded by SEQ ID NO: 135, such at least 80%, at least 90%, at least 95%, at least 99% or 100% identity to the amino acid sequence encoded by SEQ ID NO: 135. In some cases, the CD28 activation domain comprises or consists of an amino acid sequence having at least 70% identity to the amino acid sequence encoded by SEQ ID NO: 136, such at least 80%, at least 90%, at least 95%, at least 99% or 100% identity to the amino acid sequence encoded by SEQ ID NO: 136. In some cases, the CD3zeta activation domain comprises or consists of an amino acid sequence having at least 70% identity to the amino acid sequence encoded by any one of SEQ ID NOs: 137-139, such at least 80%, at least 90%, at least 95%, at least 99% or 100% identity to the amino acid sequence encoded by any one of SEQ ID NOs: 137-139. For example, the 4-1BBz domain may comprise an amino acid sequence having at least 70% identity to the amino acid sequence encoded by SEQ ID NO: 135 (such at least 80%, at least 90%, at least 95%, at least 99% or 100% identity to the amino acid sequence encoded by SEQ ID NO: 135) and an amino acid sequence having at least 70% identity to the amino acid sequence encoded by any one of SEQ ID NOs: 137-139 (such at least 80%, at least 90%, at least 95%, at least 99% or 100% identity to the amino acid sequence encoded by any one of SEQ ID NOs: 137-139). Similarly, a CD28z domain may comprise an amino acid sequence having at least 70% identity to the amino acid sequence encoded by SEQ ID NO: 136 (such at least 80%, at least 90%, at least 95%, at least 99% or 100% identity to the amino acid sequence encoded by SEQ ID NO: 136) and an amino acid sequence having at least 70% identity to the amino acid sequence encoded by any one of SEQ ID NOs: 137-139 (such at least 80%, at least 90%, at least 95%, at least 99% or 100% identity to the amino acid sequence encoded by any one of SEQ ID NOs: 137-139).

The transmembrane domain spans the cell membrane, for example the cell membrane of a eukaryotic cell. The transmembrane domain serves to transmit activation signals to the cytoplasmic signal transduction domains following ligand binding of the extracellular antigen-binding domains (e.g. scFv). The transmembrane domain may be derived from a naturally occurring transmembrane protein, such as a type-I transmembrane protein. The transmembrane domain is typically the transmembrane domain of CD28 or CD8α. The transmembrane domain may be a transmembrane domain of the α, β, δ or γ subunits of the T-cell receptor, CD38, CD35, CD4, CD6, CD8α, CD28, CD86, OX-40, 4-1BB or CD40L (CD154). The transmembrane domain may be the transmembrane domain of CD8, for example, when the immune effector cell is an NK cell.

In some cases, the CD8a transmembrane domain comprises or consists of an amino acid sequence having at least 70% identity to SEQ ID NO: 122, such at least 80%, at least 90%, at least 95%, at least 99% or 100% identity to SEQ ID NO: 122. In some cases, the CD28 transmembrane domain comprises or consists of an amino acid sequence having at least 70% identity to SEQ ID NO: 123, such at least 80%, at least 90%, at least 95%, at least 99% or 100% identity to SEQ ID NO: 123.

In some cases, the CD8α transmembrane domain comprises or consists of an amino acid sequence having at least 70% identity to the amino acid sequence encoded by SEQ ID NO: 133, such at least 80%, at least 90%, at least 95%, at least 99% or 100% identity to the amino acid sequence encoded by SEQ ID NO: 133. In some cases, the CD28 transmembrane domain comprises or consists of an amino acid sequence having at least 70% identity to the amino acid sequence encoded by SEQ ID NO: 134, such at least 80%, at least 90%, at least 95%, at least 99% or 100% identity to the amino acid sequence encoded by SEQ ID NO: 134.

The CAR may comprise a hinge that connects the transmembrane domain to the extracellular domain. The hinge may confer steric effects that influence the strength of activation, cytotoxicity and signalling from the target cell and its surface receptors. In some cases, the hinge may be from a region from another immune molecule such as IgG1, IgG2, IgG3, IgG4, CD8 (e.g. a CD8α hinge) or CD28. The hinge may be any suitable length. The hinge may be at least one amino acid in length, such as at least five, at least 10 or at least 20 amino acids in length. The hinge may be one hundred or fewer amino acids in length, such as 80 or fewer, 60 or fewer, 40 or fewer, 30 or fewer, or 20 or fewer amino acids in length. The hinge may be 1 to 40 amino acids in length, such as 2 to 30, 3 to 25, 4 to 20, or 5 to 15 amino acids in length. The hinge is may comprises glycine and serine, threonine and/or alanine residues. However, the hinge may comprise any suitable residues. In some cases, the hinge is an IgG1 hinge, such as a human IgG1 hinge.

In some cases, the hinge is an IgG1 hinge (e.g. human IgG1 hinge) further comprising one or more constant domains, such as CH2 or CH3.

In some cases, the hinge is an IgG4 hinge, such as a human IgG4 hinge.

In some cases, the hinge is a CD8α hinge, such as a human CD8α hinge.

In some cases, the CD8α hinge comprises or consists of an amino acid sequence having at least 70% identity to SEQ ID NO: 117, such at least 80%, at least 90%, at least 95%, at least 99% or 100% identity to SEQ ID NO: 117. In some cases, the IgG4 hinge comprises or consists of an amino acid sequence having at least 70% identity to SEQ ID NO: 118, such at least 80%, at least 90%, at least 95%, at least 99% or 100% identity to SEQ ID NO: 118. The IgG4 hinge set out in SEQ ID NO: 118 is otherwise referred to herein as the ‘short’ hinge. In some cases, the IgG1 hinge comprises or consists of an amino acid sequence having at least 70% identity to any one of SEQ ID NOs: 119 to 121, such at least 80%, at least 90%, at least 95%, at least 99% or 100% identity to any one of SEQ ID NOs: 119 to 121. The IgG1 hinge set out in SEQ ID NO: 121 is otherwise referred to herein as the ‘long’ hinge.

In some cases, the CD8α hinge comprises or consists of an amino acid sequence having at least 70% identity to the amino acid sequence encoded by SEQ ID NO: 129, such at least 80%, at least 90%, at least 95%, at least 99% or 100% identity to the amino acid sequence encoded by SEQ ID NO: 129. In some cases, the IgG4 hinge comprises or consists of an amino acid sequence having at least 70% identity to the amino acid sequence encoded by SEQ ID NO: 130, such at least 80%, at least 90%, at least 95%, at least 99% or 100% identity to the amino acid sequence encoded by SEQ ID NO: 130. In some cases, the IgG1 hinge comprises or consists of an amino acid sequence having at least 70% identity to the amino acid sequence encoded by SEQ ID NO: 131 or 132, such at least 80%, at least 90%, at least 95%, at least 99% or 100% identity to the amino acid sequence encoded by SEQ ID NO: 131 or 132.

The CAR may comprise a transmembrane domain and a hinge derived from the same source. For example, the transmembrane domain and the hinge may be a CD8α transmembrane domain and a CD8α hinge. The transmembrane domain and the hinge may be a CD28 transmembrane domain and a CD28 hinge.

In some cases, the CAR may comprise a ‘short’ 28z construct, i.e. comprising an IgG4 hinge and a CD28z intracellular domain. The CD28z domain may comprise an amino acid sequence having at least 70% identity to SEQ ID NO: 125 (such at least 80%, at least 90%, at least 95%, at least 99% or 100% identity to SEQ ID NO: 125) and an amino acid sequence having at least 70% identity to any one of SEQ ID NOs: 126-128 (such at least 80%, at least 90%, at least 95%, at least 99% or 100% identity to any one of SEQ ID NOs: 126-128). The IgG4 hinge may comprises or consists of an amino acid sequence having at least 70% identity to SEQ ID NO: 118, such at least 80%, at least 90%, at least 95%, at least 99% or 100% identity to SEQ ID NO: 118.

In some cases, the CAR may comprise a ‘long’ 28z construct, i.e. comprising the long IgG1 hinge (comprising the CH2 and CH3 domains) and a CD28z intracellular domain. The CD28z domain may comprise an amino acid sequence having at least 70% identity to SEQ ID NO: 125 (such at least 80%, at least 90%, at least 95%, at least 99% or 100% identity to SEQ ID NO: 125) and an amino acid sequence having at least 70% identity to any one of SEQ ID NOs: 126-128 (such at least 80%, at least 90%, at least 95%, at least 99% or 100% identity to any one of SEQ ID NOs: 126-128). The IgG1 hinge may comprise or consist of an amino acid sequence having at least 70% identity to SEQ ID NO: 121, such at least 80%, at least 90%, at least 95%, at least 99% or 100% identity to SEQ ID NO: 121.

The ‘short’ and ‘long’ 28z constructs typically comprise the CD28 transmembrane domain. For example, the short′ and ‘long’ 28z constructs may further comprise an amino acid sequence having at least 70% identity to SEQ ID NO: 123, such at least 80%, at least 90%, at least 95%, at least 99% or 100% identity to SEQ ID NO: 123.

The CAR may comprise more than one extracellular antigen-binding domain, such as two extracellular antigen-binding domains or three extracellular antigen-binding domains. The two or more extracellular antigen binding domains may bind to different glioma associated antigens, i.e. the CAR may be bispecific or multispecific.

In some cases, the invention relates to a CAR specific for PTPRZ1. In some cases, the invention relates to a CAR specific for BCAN. The inventors have surprisingly shown for the first time that an immune effector cell expressing a CAR specific for PTPRZ1 or BCAN has therapeutic utility, specifically for the treatment of glioblastoma. Any antigen-binding domain specific for PTPRZ1 or for BCAN may be used in the CAR, such as those known in the art. Preferably, the antigen-binding domain is an antigen-binding domain disclosed herein.

Antigen-binding domain The CARs discussed herein comprise an antigen-binding domain. The antigen-binding domain may be any domain that specifically binds to a glioma-associated antigen. For example, the antigen-binding domain may be an scFv, a monoclonal antibody (comprising 2 heavy chains and 2 light chains), a polyclonal antibody, Fab, a Fab′, a F (ab′) 2 fragment, a heavy chain variable domain (VH) or a nanobody (VHH).

In some cases, the antigen-binding domain comprises one or more immunoglobulin variable domains. For example, the CAR may comprise one or more immunoglobulin variable domains. Each immunoglobulin variable domain typically comprises three complementarity determining regions (CDRs) or antigen binding regions (ABRs) (these terms are used interchangeably herein). The CDRs or ABRs typically are responsible for antigen specificity, for example, by making direct interactions with the antigen. The immunoglobulin variable domains also comprise framework regions, which provide the immunoglobulin-like structure of the domain and typically do not make direct interactions with an antigen. The immunoglobulin variable domains may be selected from an immunoglobulin variable domain from an scFv domain, an antibody domain (e.g. VH and/or VL) domains, typically human, a Fab, a Fab′, a F(ab′) 2 fragment, a VHH domain and a VNAR domain. Accordingly, the antigen-binding domain may comprise an scFv domain, which typically comprises two immunoglobulin variable domains (e.g. a VH and a VL). In some cases, the antigen-binding domain may comprise a VHH domain, which typically comprises a single immunoglobulin variable domain.

Preferably, the antigen-binding domain is an scFv or a VHH domain. ScFv domains comprise a heavy chain variable domain (VH) and a light chain variable domain (VL) of an immunoglobulin and connected by a short linker peptide. Exemplary scFv domains of the invention are shown in SEQ ID NOs: 21-40. The scFv may be derived from a human immunoglobulin. The scFv may be a derived from a murine immunoglobulin.

Whilst an scFv is typically arranged VH-VL in an N-terminal to C-terminal orientation, antigen binding regions arranged VL-VH in an N-terminal to C-terminal orientation are also encompassed according to the invention.

Any linker may be used to link the (VH) and (VL) domains. In SEQ ID NOs: 21-40, the linker GGGGSGGGGSGGGGS (SEQ ID NO: 81) has been used. The linker may comprise SSSGGGGSGGGGSGGGGSS (SEQ ID NO: 82).

In some cases, a CAR described herein is selected from a CAR comprising an amino acid sequence having at least 70%, at least 80%, at least 90%, at least 95%, at least 96%, at least 97%, at least 98%, at least 99% or 100% sequence identity to the amino acid sequence of any one of SEQ ID NOs: 21 to 40.

In some cases, a CAR described herein comprises an scFv domain, comprising a VH domain having an amino acid sequence having at least 70%, at least 80%, at least 90%, at least 95%, at least 96%, at least 97%, at least 98%, at least 99% or 100% sequence identity to an amino acid selected from SEQ ID NOs: 41, 43, 45, 47, 49, 51, 53, 55, 57, 59, 61, 63, 65, 67, 69, 71, 73, 75, 77 and 79, and a VL domain having at least 70%, at least 80%, at least 90%, at least 95%, at least 96%, at least 97%, at least 98%, at least 99% or 100% sequence identity to an amino acid selected from SEQ ID NOs: 42, 44, 46, 48, 50, 52, 54, 56, 58, 60, 62, 64, 66, 68, 70, 72, 74, 76, 78 and 80, respectively. The scFv further comprises a linker sequence. The linker sequence may be, for example, any linker sequence discussed above.

In some cases, a CAR described herein comprises an scFv domain having the antigen-binding regions (ABRs) of the VH and VL domains of an scFv selected from SEQ ID NOs: 21-40. The framework regions of the VH and VL domains are the regions of the scFv outside of the ABRs and the linker sequence. In some cases, a CAR described herein comprises an scFv domain having the ABRs of the VH and VL domains of an scFv selected from SEQ ID NOs: 21-40 and at least 70%, at least 80%, at least 90%, at least 95%, at least 96%, at least 97%, at least 98%, at least 99% or 100% sequence identity to the scFv selected from SEQ ID NOs: 21-40. The term “antigen-binding regions” in an scFv is used to refer to the complementary determining regions (CDR) of the variable heavy chain and variable light chain domains that make up the scFv. For example, the scFvs of SEQ ID NOs: 21-40 are labelled with six ABRs. For each scFv, the first ABR1, ABR2 and ABR3 correspond to the heavy chain CDR1, CDR2 and CDR3 respectively, and the second set of ABR1, ABR2 and ABR3 within the sequence correspond to the light chain CDR1, CDR2 and CDR3.

In some cases, a CAR described herein is selected from a CAR comprising a polypeptide, such as one or more polypeptides, comprising the heavy chain complementarity determining regions (HCDR1, HCDR2 and HCDR3) and the light chain complementarity determining regions (LCDR1, LCDR2 and LCDR3) from an amino acid sequence selected from any one of SEQ ID NOs: 21-40. In some cases, the CAR is selected from a CAR comprising a polypeptide, such as one or more polypeptides, comprising HCDR1-3 and LCDR1-3 of an amino acid sequence selected from any one of SEQ ID NOs: 21-40 and at least 70%, at least 80%, at least 90%, at least 95%, at least 96%, at least 97%, at least 98%, at least 99% or 100% sequence identity to the polypeptide selected from any one of SEQ ID NOs: 21-40. Methods and techniques for identifying CDRs within HCVR and LCVR amino acid sequences are well known in the art and can be used to identify CDRs within the specified sequenced disclosed herein. Specifically, the scFv sequences shown in SEQ ID NOs: 21-40 comprise, from N-terminal to C-terminal, a heavy chain variable region of an antibody, a linker and a light chain variable region of an antibody. The separate heavy and light chain variable regions of SEQ ID NOs 21-40 are provided in SEQ ID NOs: 41-80. Exemplary conventions that can be used to identify the boundaries of CDRs include the Kabat definition, the Chothia definition and the IMGT definition (see, for example, Kabat, Elvin Abraham. Sequences of proteins of immunological interest. No. 91. US Department of Health and Human Services, Public Health Service, National Institutes of Health, 1991; Lefranc, Marie-Paule, et al. “IMGT unique numbering for immunoglobulin and T cell receptor variable domains and Ig superfamily V-like domains.” Developmental & Comparative Immunology 27.1 (2003): 55-77). The CDRs may be the ABRs identified in the informal sequence listing provided above. For example, the heavy chain CDR1, CDR2 and CDR3 may be the first (i.e. preceding the linker) ABR1, ABR2 and ABR3 identified for an scFV, respectively, and the light chain CDR1, CDR2 and CDR3 may be the second (i.e. following the linker) ABR1, ABR2 and ABR3 identified for the scFv, respectively.

An scFv having the ABRs of SEQ ID NO: 21, or polypeptides having the CDRs of SEQ ID NOs 41 and 42, comprises a first ABR1 (HCDR1) having the sequence FTFSSYAMH (SEQ ID NO: 105), a first ABR2 (HCDR2) having the sequence WVAVISYDGSNKYY (SEQ ID NO: 106), a first ABR3 (HCDR3) having the sequence RGSGYSYGPGYDAFDI (SEQ ID NO: 107), a second ABR1 (LCDR1) having the sequence SGSIASNYVQ (SEQ ID NO: 108), a second ABR2 (LCDR2) having the sequence TTVIYEDNQRPS (SEQ ID NO: 109), and a second ABR3 (LCDR3) having the sequence QSWDPVFG (SEQ ID NO: 110). Similarly, an scFv having the ABRs of SEQ ID NO: 22, or polypeptides having the CDRs of SEQ ID NOs 43 and 44, comprises a first ABR1 (HCDR1) having the sequence GTFSSYAIS (SEQ ID NO: 111), a first ABR2 (HCDR2) having the sequence WMGGIIPIFGTANY (SEQ ID NO: 112), a first ABR3 (HCDR3) having the sequence REGGAVGYYYGMDV (SEQ ID NO: 113), a second ABR1 (LCDR1) having the sequence SSDVGGYNYVS (SEQ ID NO: 114), a second ABR2 (LCDR2) having the sequence LMIYEVSNRPS (SEQ ID NO: 115), and a second ABR3 (LCDR3) having the sequence SSYDRSNRSM (SEQ ID NO: 116). The same applies to any of SEQ ID NOs 21-80 and similar considerations apply to CDRs 1-3 of each of SEQ ID NOs: 83 to 93.

The antigen-binding domain may be an scFv or a VHH (otherwise known as a VHH, a nanobody, a sdAb or a single domain antibody; said terms are used interchangeably herein).

In some cases, the antigen-binding domain is preferably a VHH. VHH domains comprise a single heavy chain variable domain (VH) and lack the constant domain found in typical antibodies. Exemplary VHH domains of the invention are shown in SEQ ID NOS: 83-93.

In some cases, a CAR described herein comprises a polypeptide comprising the heavy chain complementarity determining regions (HCDR1, HCDR2 and HCDR3) and the light chain complementarity determining regions (LCDR1, LCDR2 and LCDR3) from an amino acid sequence selected from any one of SEQ ID NOs: 21-40. The polypeptide may comprise two immunoglobulin variable domains, for example, may comprise an scFv. In some cases, a CAR described herein comprises a polypeptide, typically a VHH, comprising the complementary determining regions (CDR1, CDR2 and CDR3) from an amino acid sequence selected from any one of SEQ ID NOs: 83-93.

In some cases, the CAR comprises an amino acid sequence having at least 70%, at least 80%, at least 90%, at least 95%, at least 96%, at least 97%, at least 98%, at least 99% or 100% sequence identity to the amino acid sequence of any one of SEQ ID NOs: 21 to 40 and 83-93. Typically, the CAR comprises the exact CDRs of the SEQ ID NOs from which the CAR is derived.

### Glioma-Associated Antigens

Glioma-associated antigens are antigens associated with malignant glioma (glioblastoma, GBM), e.g. expressed by cells of a malignant glioma. The glioma-associated antigens may be human glioma-associated antigens. An antigen is associated with malignant glioma when it is over-expressed in a malignant glioma sample when compared to normal brain tissues and non-CNS normal tissues. The glioma-associated antigen may be associated with gliomagenesis.

The glioma-associated antigens may be selected from receptor-type tyrosine-protein phosphatase zeta (PTPRZ1), brevican core protein (BCAN), chondroitin sulfate proteoglycan 4 (CSPG4) and tenascin (TNC).

PTPRZ1, CSPG4 and BCAN may each be considered a cell surface (glioma) marker. TNC and BCAN may each be considered an extracellular matrix (ECM) marker, e.g. of tumour invasiveness. In some cases, it is advantageous for a CAR T cell of the invention to be specific to a cell surface marker and an ECM marker, such as the markers described herein.

Exemplary scFvs specific for PTPRZ1 are set out in SEQ ID NOs: 21-26. The corresponding VH and VL domains are set out in SEQ ID NOs 41-52. Preferably, a CAR as described herein may be based on the scFv domain of RRB470, RRB471 or RRB476 (SEQ ID NOs 22, 23 and 26, respectively). For example, the CAR may be selected from a CAR comprising (a) a polypeptide having the amino acid sequence of any one of SEQ ID NOs: 22, 23 or 26, (b) an amino acid sequence with at least 80%, 85%, 90%, 95% or 99% identity thereto, or (c) the HCDR1, HCDR2 and HCDR3 and the LCDR1, LCDR2 and LCDR3 from an amino acid sequence selected from any one of SEQ ID NOs: 22, 23 or 26.

Exemplary VHHS specific for PTPRZ1 are set out in SEQ ID NOs: 89-91. Preferably, a CAR described herein may be based on the VHH domain of RB832, RB833 or RB 834 (SEQ ID NO: 89, 90 and 91, respectively). For example, the CAR may be selected from a CAR comprising a polypeptide (such as an immunoglobulin variable domain and/or a VHH) comprising CDR1, CDR2 and CDR3 from an amino acid sequence selected from any one of SEQ ID NOs: 89 to 91. The CAR may be selected from an amino acid sequence with at least 80%, 85%, 90%, 95% or 99% identity to an amino acid sequence selected from any one of SEQ ID NOs: 89 to 91, typically comprising CDRs 1-3 of SEQ ID NOs: 89-91 respectively. The CAR may be selected from a CAR comprising the amino acid sequence of any one of SEQ ID NOs: 89-91.

Exemplary scFvs specific for BCAN are set out in SEQ ID NOs: 34-47. The corresponding VH and VL domains are set out in SEQ ID NOs 67-74. In some cases, a scFv specific for BCAN is selected from RBR295 (SEQ ID NO: 35). For example, the CAR may be selected from a CAR comprising (a) a polypeptide having the amino acid sequence of any one of SEQ ID NOs: 34-47, (b) an amino acid sequence with at least 80%, 85%, 90%, 95% or 99% identity thereto, or (c) the HCDR1, HCDR2 and HCDR3 and the LCDR1, LCDR2 and LCDR3 from an amino acid sequence selected from any one of SEQ ID NOs: 34-47.

Exemplary VHHS specific for BCAN are set out in SEQ ID NOs: 83-86. Preferably, a CAR described herein may be based on the VHH domain of RB826, RB827, RB828 or RB829 (SEQ ID NOs: 83-86 respectively). For example, the CAR may be selected from a CAR comprising a polypeptide (such as an immunoglobulin variable domain and/or a VHH) comprising CDR1, CDR2 and CDR3 from an amino acid sequence selected from any one of SEQ ID NOs: 83-86. The CAR may be selected from an amino acid sequence with at least 80%, 85%, 90%, 95% or 99% identity to an amino acid sequence selected from any one of SEQ ID NOs: 83-86, typically comprising CDRs 1-3 of SEQ ID NOs: 83-86 respectively. The CAR may be selected from a CAR comprising the amino acid sequence of any one of SEQ ID NOs: 83-86.

Exemplary scFvs specific for CSPG4 are set out in SEQ ID NOs: 27-33. The corresponding VH and VL domains are set out in SEQ ID NOs 53-66. Preferably, a CAR as described herein may be based on the scFv domain of HRB299, HRB 301, HRB302 or HRB 303 (SEQ ID NOs 28 and 30-32, respectively). For example, the CAR may be selected from a CAR comprising (a) a polypeptide having the amino acid sequence of any one of SEQ ID NOs: 28, 30, 31 or 32, (b) an amino acid sequence with at least 80%, 85%, 90%, 95% or 99% identity thereto, or (c) the HCDR1, HCDR2 and HCDR3 and the LCDR1, LCDR2 and LCDR3 from an amino acid sequence selected from any one of SEQ ID NOs: 28, 30, 31 or 32. More preferably, a CAR as described herein may be based on the scFv domain of HRB301 or HRB302 (SEQ ID NOs 30-31, respectively). For example, the CAR may be selected from a CAR comprising (a) a polypeptide having the amino acid sequence of SEQ ID NO: 30 or 31, (b) an amino acid sequence with at least 80%, 85%, 90%, 95% or 99% identity thereto, or (c) the HCDR1, HCDR2 and HCDR3 and the LCDR1, LCDR2 and LCDR3 from an amino acid sequence selected from SEQ ID NO: 30 or 31.

Exemplary VHHS specific for CSPG4 are set out in SEQ ID NOs: 87-88. Preferably, a CAR described herein may be based on the VHH domain of RB830 or RB831 (SEQ ID NOs: 87-88 respectively). For example, the CAR may be selected from a CAR comprising a polypeptide (such as an immunoglobulin variable domain and/or a VHH) comprising CDR1, CDR2 and CDR3 from an amino acid sequence selected from any one of SEQ ID NOs: 87-88. The CAR may be selected from an amino acid sequence with at least 80%, 85%, 90%, 95% or 99% identity to an amino acid sequence selected from any one of SEQ ID NOs: 87-88, typically comprising CDRs 1-3 of SEQ ID NOs: 87-88 respectively. The CAR may be selected from a CAR comprising the amino acid sequence of any one of SEQ ID NOs: 87-88.

Exemplary scFvs specific for TNC are set out in SEQ ID NOs: 38-40. The corresponding V_{H} and V_{L} domains are set out in SEQ ID NOs 75-80. For example, the CAR may be selected from a CAR comprising (a) a polypeptide having the amino acid sequence of any one of SEQ ID NOs: 38-40, (b) an amino acid sequence with at least 80%, 85%, 90%, 95% or 99% identity thereto, or (c) the HCDR1, HCDR2 and HCDR3 and the LCDR1, LCDR2 and LCDR3 from an amino acid sequence selected from any one of SEQ ID NOs: 38-40.

Exemplary VHHS specific for TNC are set out in SEQ ID NOs: 92-93. Preferably, a CAR described herein may be based on the VHH domain of RB835 or RB836 (SEQ ID NOs: 92-93 respectively). For example, the CAR may be selected from a CAR comprising a polypeptide (such as an immunoglobulin variable domain and/or a VHH) comprising CDR1, CDR2 and CDR3 from an amino acid sequence selected from any one of SEQ ID NOs: 92-93. The CAR may be selected from an amino acid sequence with at least 80%, 85%, 90%, 95% or 99% identity to an amino acid sequence selected from any one of SEQ ID NOs: 92-93, typically comprising CDRs 1-3 of SEQ ID NOs: 92-93 respectively. The CAR may be selected from a CAR comprising the amino acid sequence of any one of SEQ ID NOs: 92-93.

Other known glioma-associated antigens include HER2, EGFRvIII, IL13Ra2, PDGFRA, NKG2D, MET, HGF, B7-H3. Preferably, other glioma-associated antigens are selected from HER2, EGFRvIII and IL13Ra2, more preferably Her2 and IL13Ra2.

A monospecific CAR may be specific for any one of the above glioma associated antigens. A bispecific or multispecific CAR may be specific for any combination of glioma associated antigens. Preferably, a bispecific or multispecific CAR is specific for at least one of PTPRZ1, BCAN, CSPG4, and TNC, such as two, three or all four of PTPRZ1, BCAN, CSPG4, and TNC. The bispecific or multispecific CAR may be additionally specific for other known glioma associated antigens, such as those described herein. For example, a multispecific CAR may be specific for PTPRZ1 and CSPG4; CSPG4 and Her2; CSPG4 and IL13Ra2; CSPG4, Her2 and IL13Ra2; PTPRZ1 and Her2; PTPRZ1 and IL13Ra2; or PTPRZ1, Her2 and IL13Ra2.

In some cases, a bispecific or multispecific CAR is specific for PTPRZ1 and one or more other glioma-associated antigens. In some cases, a bispecific or multispecific CAR is specific for BCAN and one or more other glioma-associated antigens.

A population of immune effector cells may comprise two or more different immune effector cells, wherein each different immune effector cell is specific for a different glioma associated antigen. Preferably, the population comprise at least one immune effector cell specific for at least one of PTPRZ1, BCAN, CSPG4, and TNC, such as two immune effector cells specific for two of PTPRZ1, BCAN, CSPG4, and TNC, three immune effector cells specific for three of PTPRZ1, BCAN, CSPG4, and TNC, or four immune effector cells specific for all of PTPRZ1, BCAN, CSPG4, and TNC. The population may comprise additional immune effector cell specific for other known glioma-associated antigens. For example, a population of immune effector cells may comprise an immune effector cell specific for PTPRZ1 and an immune effector cell specific for CSPG4. A population of immune effector cells may comprise an immune effector cell specific for PTPRZ1, an immune effector cell specific for Her2 and an immune effector cell specific for IL13Ra2, as illustrated in the Examples.

In some cases, a population of immune effector cells may comprise two or more difference immune effector cells, wherein one or more of the different immune effector cells is specific for a glioma-associated antigen selected from at least one of PTPRZ1, BCAN, CSPG4, and TNC, such at least two of PTPRZ1, BCAN, CSPG4, and TNC, at least three of PTPRZ1, BCAN, CSPG4, and TNC, or all four of PTPRZ1, BCAN, CSPG4, and TNC. In some cases, one or more of the different immune effector cells is specific for PTPRZ1. In some cases, one or more of the different immune effector cells is specific for BCAN. For example, a population of immune effector cells may comprise: an immune effector cell specific for PTPRZ1 and an immune effector cell specific for BCAN; an immune effector cell specific for PTPRZ1 and an immune effector cell specific for CSPG4; an immune effector cell specific for PTPRZ1 and an immune effector cell specific for TNC; an immune effector cell specific for BCAN and an immune effector cell specific for CSPG4; an immune effector cell specific for BCAN and an immune effector cell specific for TNC; an immune effector cell specific for CSPG4 and an immune effector cell specific for TNC; an immune effector cell specific for PTPRZ1, an immune effector cell specific for BCAN and an immune effector cell specific for CSPG4; an immune effector cell specific for PTPRZ1, an immune effector cell specific for BCAN and an immune effector cell specific for TNC; an immune effector cell specific for PTPRZ1, an immune effector cell specific for CSPG4 and an immune effector cell specific for TNC; an immune effector cell specific for BCAN, an immune effector cell specific for CSPG4 and an immune effector cell specific for TNC; or an immune effector cell specific for PTPRZ1, an immune effector cell specific for BCAN, an immune effector cell specific for CSPG4 and an immune effector cell specific for TNC.

In some cases, an immune effector cell of the invention may comprise two or more different CARs, wherein each CAR is specific for a different glioma associated antigen. Preferably, the immune effector cell comprises at least one CAR specific for at least one of PTPRZ1, BCAN, CSPG4, and TNC, such as two CARs specific for two of PTPRZ1, BCAN, CSPG4, and TNC, three CARs specific for three of PTPRZ1, BCAN, CSPG4, and TNC, or four CARs specific for all of PTPRZ1, BCAN, CSPG4, and TNC. The immune effector cell may comprise additional CARs specific for other known glioma-associated antigens as described herein. For example, an immune effector cell may comprise a CAR specific for PTPRZ1 and a CAR specific for CSPG4. An immune effector cell may comprise a CAR specific for PTPRZ1, a CAR specific for Her2 and a CAR specific for IL13Ra2.

In some cases, an immune effector cell of the invention may comprise two or more different CARs, wherein each CAR is specific for a different glioma associated antigen. In some cases, at least one of the glioma-associated antigens is PTPRZ1. In some cases, at least one of the glioma-associated antigens is BCAN. In some cases, at least one of the glioma-associated antigens is selected from the group of PTPRZ1, BCAN, CSPG4 and TNC, such at least two of PTPRZ1, BCAN, CSPG4, and TNC, at least three of PTPRZ1, BCAN, CSPG4, and TNC, or all four of PTPRZ1, BCAN, CSPG4, and TNC. For example, an immune effector cells may comprise: a CAR specific for PTPRZ1 and a CAR specific for BCAN; a CAR specific for PTPRZ1 and a CAR specific for CSPG4; a CAR specific for PTPRZ1 and a CAR specific for TNC; a CAR specific for BCAN and a CAR specific for CSPG4; a CAR specific for BCAN and a CAR specific for TNC; a CAR specific for CSPG4 and a CAR specific for TNC; a CAR specific for PTPRZ1, a CAR specific for BCAN and a CAR specific for CSPG4; a CAR specific for PTPRZ1, a CAR specific for BCAN and a CAR specific for TNC; a CAR specific for PTPRZ1, a CAR specific for CSPG4 and a CAR specific for TNC; a CAR specific for BCAN, a CAR specific for CSPG4 and a CAR specific for TNC; or a CAR specific for PTPRZ1, a CAR specific for BCAN, a CAR specific for CSPG4 and a CAR specific for TNC. The immune effector cell may comprise additional CARs specific for other known glioma-associated antigens as described herein.

### Nucleic Acids

Also provided is one or more isolated nucleic acids (i.e. polynucleotides) encoding the CAR of the invention. In some cases, the encoding nucleic acid sequence may be provided by more than one nucleic acid sequence, optionally present on more than one nucleic acid molecule, but collectively together they are able to encode a CAR of the invention.

Nucleic acids which encode a CAR of the invention can be obtained by methods well known to those skilled in the art. For example, DNA sequences coding for part or all of the antibody heavy and light chains may be synthesised as desired from the corresponding amino acid sequences.

The nucleic acid may be a DNA sequence. The nucleic acid may be an RNA sequence, such as mRNA. A vector may comprise the nucleic acid.

The vector may be a viral vector. Conventional viral based expression systems could include retroviral, alpha-retroviral, lentivirus, adenoviral, adeno-associated (AAV) and herpes simplex virus (HSV) vectors for gene transfer. Non-viral transduction vectors include transposon-based systems including PiggyBac and Sleeping Beauty systems. Methods for producing and purifying such vectors are known in the art.

The vectors may be cloning vectors or expression vectors. A suitable vector may be any vector which is capable of carrying a sufficient amount of genetic information, and allowing expression of a polypeptide of the invention.

The vector is preferably an RNA vector. Suitable RNA vectors include the RNA vectors described in Schutsky, Keith, et al., Oncotarget 6.30 (2015): 28911 and Beatty, Gregory L., et al., Gastroenterology 155.1 (2018): 29-32.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

A nucleic acid may be provided in the form of an expression cassette, which includes control sequences operably linked to the inserted sequence, thus allowing for expression of the CAR of the invention in vivo. Hence, also provided is one or more expression cassettes encoding the one or more nucleic acids that encode a CAR described herein. These expression cassettes, in turn, are typically provided within vectors (e.g. plasmids or recombinant viral vectors). Hence, also provided is a vector encoding a CAR described herein. Further provided are vectors which collectively encode a CAR described herein.

The vector may be a human artificial chromosome. Human artificial chromosomes are described in e.g. Kazuki et al., Mol. Ther. 19 (9): 1591-1601 (2011), and Kouprina et al., Expert Opinion on Drug Delivery 11 (4): 517-535 (2014).

The vector may be a non-viral delivery system, such as DNA plasmids, naked nucleic acid (e.g. naked RNA), and nucleic acid complexed with a delivery vehicle, such as a liposome or a nanoparticle.

The nucleic acids, expression cassettes or vectors described herein may be introduced into a host cell, e.g. by transfection. Hence, also provided is a host cell comprising the one or more nucleic acids, expression cassettes or vectors of the invention. The nucleic acids, expression cassettes or vectors described herein may be introduced transiently or permanently into the host cell, allowing expression of an antibody from the one or more nucleic acids, expression cassettes or vectors. Such host cells include transient, or preferably stable higher eukaryotic cell lines, such as mammalian cells or insect cells, lower eukaryotic cells, such as yeast, or prokaryotic cells, such as bacteria cells. Particular examples of cells include mammalian HEK293, such as HEK293F, HEK293T, HEK293S or HEK Expi293F, CHO, HeLa, NSO and COS cells, or any other cell line used herein. Preferred host cells are the immune effector cells described herein. Preferably, the nucleic acids, expression cassettes or vectors described herein are introduced transiently into the host cell.

Also provided is a kit suitable for transforming and/or transfecting an immune effector cell or a population of immune effector cells to generate an immune effector cell or population of immune effector cells of the invention. The kit comprises a nucleic acid or vector described herein. The kit may comprise further agents such as those discussed herein that improve transfection or transformation efficacy.

Also described are nucleic acids encoding the specific scFv antigen binding regions of SEQ ID NOs: 21-40, which are provided in SEQ ID NOs: 1-20, respectively. Whilst SEQ ID NOs: 1-20 are provided as a DNA sequence, the corresponding RNA sequence (replacing ‘T’ with ‘U’) is also encompassed.

Also described are nucleic acids encoding the specific scFv antigen binding regions of SEQ ID NOs: 83-93, which are provided in SEQ ID NOs: 94-104, respectively. Whilst SEQ ID NOs: 94-104 are provided as a DNA sequence, the corresponding RNA sequence (replacing ‘T’ with ‘U’) is also encompassed.

### Immune Effector Cell

Reference to an immune effector cell as used herein is a cell capable of cell-mediated cytotoxicity against a target cell displaying a target antigen, e.g. a glioma-associated antigen. The immune effector cell may be a T cell, a γδ T cell, a natural killer (NK) cell, an NKT cell, an induced pluripotent stem cell (iPSC) derived NK cell (iPSC-NK), a γδ T cell, a phagocyte, or a macrophage. The immune effector cell is preferably a T cell.

Preferably, the T cell is a CD8+ T cell, or cytotoxic T cell. The T cell is preferably a CD4-CD8+ T cell.

The T cell may be a CD4+ T cell, or helper T cell (TH cell), such as a TH1, TH2, TH3, TH17, TH9, or TFH cells. The T cell may be a regulatory T cell (Treg). The T cell may be a naïve, effector, memory, effector memory, central memory, memory stem T cell. The T cell may be a peripheral lymphocyte.

The T cell may be expanded from peripheral blood mononuclear cells (PBMCs). The T cell may be autologous with respect to a subject into which it is to be administered. The T cell may be allogeneic with respect to a subject into which it is to be administered. The T cell may be partially HLA-mismatched with respect to a subject into which it is to be administered.

The NK cell may be a cell of the NK92 cell line. The NK cell may be isolated from peripheral blood mononuclear cells (PBMCs) of the subject to be treated or of a healthy donor. The NK cell may be isolated from cord blood. The NK cell may be differentiated from a CD34+haematopoietic progenitor cell (HPC).

The γδ T cell may be expanded from peripheral blood mononuclear cells (PBMCs). The γδ T cell may be autologous with respect to a subject into which it is to be administered. The T γδ cell may be allogeneic with respect to a subject into which it is to be administered.

The macrophage may be differentiated into the “M1” phenotype. The M1 macrophage expresses pro-inflammatory cytokines and has strong anti-tumour activity. An undifferentiated macrophage expressing a CAR described herein may be induced to differentiate into the M1 phenotype by culturing in the presence of the glioma-associated antigen.

The immune effector cell may comprise a nucleic acid described herein. The immune effector cell may comprise a vector described herein. The immune effector cell preferably comprises an RNA nucleic acid or RNA vector described herein. The immune effector cell expresses a CAR specific for one or more glioma-associated antigens. The immune effector preferably transiently expresses the CAR.

Accordingly, the immune effector cell is preferably an RNA CAR T cell.

The immune effector cell may be engineered to transiently express a CAR specific for one or more glioma-associated antigens. This is to minimise on-target off-tumour toxicity as a result of expression of the antigen by normal healthy tissue. In some cases, the immune effector cell may be transfected with mRNA encoding a CAR specific for one or more glioma-associated antigens, for example by mRNA electroporation as demonstrated in Beatty et al, Gastroenterology 155.1 (2018): 29-32. (see supplementary materials 5) and Schutsky et al, Oncotarget 6.30 (2015): 28911.

In some cases, the invention relations to a population of immune effector cells that express a CAR. The population may comprise at least two different CAR-expressing immune effector cells specific for at least two different glioma-associated antigens. The population may comprise at least three different CAR-expressing immune effector cells specific for at least three different glioma-associated antigens. In some cases, one or more of the glioma-associated antigens are selected from PTPRZ1, BCAN, CSPG4 and TNC.

For example, two or more of the glioma-associated antigens, such as three or four, are selected from PTPRZ1, BCAN, CSPG4 and TNC.

A population may comprise at least about 1×10⁶ of the immune effector cells, such as at least about 1×10⁷, at least about 1×10⁸, at least about 1×10⁹ or at least about 1×10¹⁰ of the immune effector cells. A population may comprise at least about 1×10⁶ to about 1×10¹² of the immune effector cells, such as about 1×10⁶ to about 1×10¹¹, about 1×10⁶ to about 1×10¹⁰, about 1×10⁶ to about 1×10⁹, about 1×10⁷ to about 1×10¹¹, about 1×10⁸ to about 1×10¹⁰ of the immune effector cells. The population may comprise about 1×10⁶ of the immune effector cells, such as about 5×10⁶, about 1×10⁷, about 5×10⁷, about 1×10⁸, about 5×10⁸, about 1×10⁹, about 5×10⁹, about 1×10¹⁰, about 5×10¹⁰, about 1×10¹¹, about 5×10¹¹, or about 1×10¹² of the immune effector cells

In some cases, an immune effector cell of the invention expresses a CAR, wherein the CAR is specific for at least two different glioma-associated antigens (i.e. a bispecific or multispecific CAR). For example, the CAR may comprise two different scFvs specific for two different glioma-associated antigens.

In some cases, an immune effector cell of the invention expresses at least two different CARs, wherein the at least two different CARs are specific for at least two different glioma-associated antigens. In some cases, an immune effector cell of the invention expresses at least three different CARs, wherein the at least three different CARS are specific for at least three different glioma-associated antigens.

Multispecificity against two or more glioma-associated antigens is advantageous for a number of reasons. For example, due to interpatient and/or inter-tumour variability, the expression of glioma-associated antigens may vary within and between subjects. Multispecificity allows a single therapy to target a wider range of tumours. Furthermore, antigen escape is the phenomenon wherein an antigen targeted by, e.g., a CAR is no longer expressed by the tumour and the therapy loses its efficacy. By targeting a number of glioma-associated antigens, it is much more difficult for the tumour to ‘escape’ the therapy. Multispecificity also enhances immune effector cell effector functions such as cytotoxicity.

Also provided is a method of making an immune effector cell of the invention or a population of immune effector cells of the invention. The method comprises transforming the cell or the population of cells with one or more nucleic acids encoding one or more CARs specific for one or more glioma-associated antigens. The CARs may be any of the CARs discussed herein. The nucleic acids may be any of the nucleic acid or vectors of the invention.

Any method known in the art may be used to transform the cell or the population with the nucleic acid. The immune effector cell may be transfected or transduced with the nucleic acid. The CAR may be introduced to the immune effector cell using a vector. The term “transduction” may be used to describe virus mediated nucleic acid transfer. A viral vector may be used to transduce the cell with the one or more constructs. Conventional viral based expression systems could include retroviral, alpha-retroviral, lentivirus, adenoviral, adeno-associated (AAV) and herpes simplex virus (HSV) vectors for gene transfer. Non-viral transduction vectors include transposon-based systems including PiggyBac and Sleeping Beauty systems. Methods for producing and purifying such vectors are known in the art. The vector is preferably a vector described herein. Immune effector cells may be transduced using any method known in the art. Transduction may be in vitro or ex vivo.

The term “transfection” may be used to describe non-virus-mediated nucleic acid transfer. The immune effector cells may be transfected using any method known in the art. Transfection may be in vitro or ex vivo. Any vector capable of transfecting immune effector cells may be used, such as conventional plasmid DNA or RNA transfection, preferably mRNA transfection. A human artificial chromosome and/or naked RNA may be used to transfect the cell with the nucleic acid sequence or nucleic acid construct. Human artificial chromosomes are described in e.g. Kazuki et al., Mol. Ther. 19 (9): 1591-1601 (2011), and Kouprina et al., Expert Opinion on Drug Delivery 11 (4): 517-535 (2014). Alternative non-viral delivery systems include DNA plasmids, naked nucleic acid, and nucleic acid complexed with a delivery vehicle, such as a liposome. Methods of non-viral delivery of nucleic acids include lipofection, microinjection, biolistics, virosomes, liposomes, immunoliposomes, polycation or lipid: nucleic acid conjugates, naked DNA, naked RNA, artificial virions, and agent-enhanced uptake of DNA.

Nanoparticle delivery systems may be used to transfect the immune effector cell with the nucleic acid sequence. Such delivery systems include, but are not limited to, lipid-based systems, liposomes, micelles, microvesicles and exosomes. With regard to nanoparticles that can deliver RNA, see, e.g., Alabi et al., Proc Natl Acad Sci USA. 2013 Aug. 6; 110 (32): 12881-6; Zhang et al., Adv Mater. 2013 Sep. 6; 25 (33): 4641-5; Jiang et al., Nano Lett. 2013 Mar. 13; 13 (3): 1059-64; Karagiannis et al., ACS Nano. 2012 Oct. 23; 6 (10): 8484-7; Whitehead et al., ACS Nano. 2012 Aug. 28; 6 (8): 6922-9 and Lee et al., Nat Nanotechnol. 2012 Jun. 3; 7 (6): 389-93. Lipid Nanoparticles, Spherical Nucleic Acid (SNA™) constructs, nanoplexes and other nanoparticles (particularly gold nanoparticles) are also contemplated as a means for delivery of a nucleic acid or vector of the invention. The immune effector cell may be transfected by electroporation. Preferably, the electroporation is mRNA electroporation. This has the advantage of allowing transient expression of the CAR.

The immune effector cell may be transfected by electroporation, such as RNA electroporation or mRNA electroporation. Where the immune effector cell expresses more than one CAR, the immune effector cell may be transfected by electroporation (e.g. RNA or mRNA electroporation) of a single polynucleotide (e.g. RNA or mRNA) or vector encoding the more than one CAR, or may be transfected by electroporation (e.g. RNA or mRNA electroporation) of two or more polynucleotides (e.g. RNA or mRNA) or vectors, each polynucleotide encoding at least one CAR. The electroporation of the two or more polynucleotides is typically performed simultaneously.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectAmine, fugene and transfectam.

### Pharmaceutical Composition

Also provided is a composition comprising an immune effector cell or population of immune effector cells of the invention. The immune effector cell or population of immune effector cells may be at least 50% of the total cells in the composition, such as at least 60%, at least 70%, at least 80%, at least 90%, at least 95%, at least 96%, at least 97%, at least 98%, at least 99% or at least 99.9% of the total cells in the composition. The total cells in the composition may consist or consist essentially of the immune effector cell or population of immune effector cells of the invention, i.e. no other cells are detectable in the composition.

The composition may comprise at least about 1×10⁶ to about 1×10¹² of the immune effector cells of the invention, such as about 1×10⁶ to about 1×10¹¹, about 1×10⁶ to about 1×10¹⁰, about 1×10⁶ to about 1×10⁹, about 1×10⁷ to about 1×10¹¹, about 1×10⁸ to about 1×10¹⁰ of the immune effector cells. The composition may comprise about 1×10⁶ of the immune effector cells of the invention, such as about 5×10⁶, about 1×10⁷, about 5×10⁷, about 1×10⁸, about 5×10⁸, about 1×10⁹, about 5×10⁹, about 1×10¹⁰, about 5×10¹⁰, about 1×10¹¹, about 5×10¹¹, or about 1×10¹² of the immune effector cells. The composition may comprise a population of the immune effector cells of the invention in the amounts described above.

The composition may be a pharmaceutical composition. The pharmaceutical composition may comprise a pharmaceutically acceptable carrier. Suitable pharmaceutically acceptable carriers comprise aqueous carriers or diluents. Examples of suitable aqueous carriers include water, buffered water and saline.

The pharmaceutical composition may include one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts.

The composition may comprise one or more additional therapeutic agent, such as a chemotherapeutic agent. The composition may comprise one or more preservative, such as an anti-fungal and/or anti-viral agent.

### Therapeutic Uses and Methods

Also described herein is use of the immune effector cell or population of immune effector cells described herein, in a method of treatment of the human or animal body by therapy, or in a diagnostic method.

For instance, also provided is a method of treating cancer in a subject, the method comprising administering to the subject an effective amount of an immune effector cell or a population of immune effector cells of the invention.

Also provided is an immune effector cell or a population of immune effector cells of the invention for use in a method of treating cancer. Also provided is a use of an immune effector cell or a population of immune effector cells for the manufacture of a medicament for the treatment of cancer.

The therapeutic uses and methods may comprise administering a therapeutically effective amount of the immune effector cell or population of immune effector cells.

Also provided is a method of formulating a composition for treating cancer, wherein said method comprises mixing an immune effector cell or population of immune effector cells of the invention with an acceptable carrier to prepare said composition.

The cancer may be glioma, such as malignant glioma. The cancer may be glioblastoma. The cancer may be a recurrent cancer, such as recurrent glioblastoma. The subject may have been previously treated for the cancer, such as using CAR cell (e.g. T-cell) approaches target EGFRvIII, IL13Ra2 and/or Her2. The cancer may be glioblastoma multiforme (GBM). The cancer may be primary glioblastoma or second glioblastoma.

The cancer may be other solid tumours expressing the glioma-associated antigen(s). For example, the cancer may be a solid tumour expressing one or more of PTPRZ1, BCAN, CSPG4 and/or TNC. The cancer may additionally express one or more of HER2, EGFRvIII, IL13Ra2, PDGFRA, NKG2D, MET, HGF, B7-H3. The immune effector cells, populations of immune effector cells, CARs and antigen-binding fragments disclosed herein may be used for treating any solid cancers expressing the glioma-associated antigen(s).

The therapeutic methods and uses may comprise, prior to treatment with an immune effector cell or population of immune effector cells of the invention, determining whether the cancer expresses a glioma-associated antigen specifically targeted by immune effector cell or population of immune effector cells of the invention. For instance, the method may comprise determining whether the cancer expresses PTPRZ1, BCAN, CSPG4 and/or TNC. The method may comprise selecting an immune effector cell or population of immune effector cells based on the expression of glioma-associated antigens by the cancer, so that the immune effector cell or population of immune effector cell is specific for the cancer. The method may comprise transfecting or transforming an immune effector cell with a nucleic acid of the invention in response to information on the expression of glioma-associated antigens by the cancer.

At least 1% of the cancer cells from the tumour or from the individual may express a glioma-associated antigen. The glioma-associated antigen is typically selected from PTPRZ1, BCAN, CSPG4 and/or TNC. For example, at least 5%, at least 10%, at least 20%, at least 30%, at least 40%, at least 50%, at least 60%, at least 70% or at least 80% of the cancer cells from the tumour or from the individual may express the glioma-associated antigen. The percentage of cells that express the glioma-associated antigen may be determined by any means known to the skilled person, such as immunohistochemistry (IHC), flow-cytometry or enzyme-linked immunosorbent assay (ELISA).

The expression of the glioma-associated antigen may have an intensity of greater than or equal to (>) 1+, such as ≥2+or ≥3+. The intensity score may be assessed by IHC staining of the tumour, with the scoring as follows: negative=no staining or staining in less than or equal to ≤10% of the cells stained; 1+=incomplete staining in ≥10% of cells stained; 2+=weak to moderate staining in ≥10% of cells stained; strong and complete staining in ≥10% of cells stained.

The therapeutic methods and uses described herein may comprise inhibiting the disease state (i.e. the cancer), for example by arresting its development and//or causing regression of the disease state until a desired end point is reached. The therapeutic methods and uses of the invention may comprise achieving a partial response, a full response by the cancer. The therapeutic methods and uses of the invention may achieve remission of the cancer.

The therapeutic methods and uses described herein may delay the growth of the cancer, arrest the growth of the cancer and/or reverse the growth of the cancer. The therapeutic methods and uses of the invention may reduce the size of the cancer by at least 10%, such as at least 20%, at least 30%, at least 40%, at least 50%, at least 60%, at least 70%, at least 80%, at least 90% or by 100%.

The therapeutic methods and uses described herein may comprise the induction of a bystander effect. The bystander effect may comprise the killing of cancer cells which do not express a glioma-associated antigen against which at least one of the CARs expressed by the immune effector cells administered to the subject in the method or use is directed.

Typically, the therapeutic methods and uses are for a human subject in need thereof. However, non-human animals such as non-human mammals are also contemplated. The non-human mammals may be rats, rabbits, sheep, pigs, cows, cats or dogs.

The dose of the immune effector cell or population of immune effector cells may vary depending on the age and size of a subject, as well as on the disease, conditions and route of administration. The immune effector cell or population of immune effector cells may be administered at a dose of about 1×10⁶ to about 1×10¹² cells, such as about 1×10⁶ to about 1×10¹¹, about 1×10⁶ to about 1×10¹⁰, about 1×10⁶ to about 1×10⁹, about 1×10⁷ to about 1×10¹¹, about 1×10⁸ to about 1×10¹⁰ cells. The immune effector cell or population of immune effector cells may be administered at a dose of about 1×10⁶ cells, such as about 5×10⁶ cells, about 1×10⁷ cells, about 5×10⁷ cells, about 1×10⁸ cells, about 5×10⁸ cells, about 1×10⁹ cells, about 5×10⁹ cells, about 1×10¹⁰ cells, about 5×10¹⁰ cells, about 1×10¹¹ cells, about 5×10¹¹ cells, or about 1×10¹² cells.

The immune effector cell or population of immune effector cells may be administered at a dose of about 1×10⁵ cells/kg to about 1×10¹¹ cells/kg, such as about 1×10⁵ cells/kg to about 1×10¹⁰ cells/kg, about 1×10⁵ cells/kg to about 1×10⁹ cells/kg, about 1×10⁵ cells/kg to about 1×10⁸ cells/kg, about 1×10⁶ cells/kg to about 1×10¹¹ cells/kg, about 1×10⁶ cells/kg to about 1×10¹⁰ cells/kg, about 1×10⁶ cells/kg to about 1×10⁹ cells/kg, about 1×10⁷ cells/kg to about 1×10¹¹ cells/kg, about 1×10⁷ cells/kg to about 1×10¹⁰ cells/kg, or about 1×10⁷ cells/kg to about 1×10⁹ cells/kg, The immune effector cell or population of immune effector cells may be administered at a dose of about 1×10⁵ cells/kg, such as about 5×10⁵ cells/kg, 1×10⁶ cells/kg, 5×10⁶ cells/kg, 1×10⁷ cells/kg, 5×10⁷ cells/kg, 1×10⁸ cells/kg, 5×10⁸ cells/kg, 1×10⁹ cells/kg, 5×10⁹ cells/kg, 1×10¹⁰ cells/kg, 5×10¹⁰ cells/kg, or 1×10¹¹ cells/kg.

The immune effector cell or population of immune effector cells may be administered as a single dose. The immune effector cell or population of immune effector cells may be administered in a multiple dose regimen. For example, the initial dose may be followed by administration of a second or plurality of subsequent doses. The second and subsequent doses may be separated by an appropriate time. For example, the doses may be administered once about every week, once about every 2 weeks, once about every 3 weeks, once about every four weeks, or once about every month.

The immune effector cell or population of immune effector cells may be administered intravenously. The immune effector cell or population of immune effector cells may be administered intracranially. The immune effector cell or population of immune effector cells may be administered intraventricularly.

The immune effector cell or population of immune effector cells may be administered with one or more additional therapy, such as one or more additional therapeutic agents. The additional therapeutic agent may be an anti-tumour agent. The additional therapeutic agent may be oncolytic viruses. The additional therapeutic agent may be a CAR-enhancing drug. The additional therapeutic may be an additional immune effector cell.

Combined administration of the immune effector cell or population with the additional therapeutic agent may be achieved in a number of different ways. All the components may be administered together in a single composition. Each component may be administered separately as part of a combined therapy.

For example, the immune effector cell or the population of immune effector cells of the invention may be administered before, after or concurrently with the additional therapeutic agent.

The additional therapy may be chemotherapy, radiotherapy and/or surgery.

Prior to administration of the immune effector cell or population of immune effector cells of the invention, the subject may undergo lymphodepletion. Lymphodepletion may be achieved via administration to the subject with fluradabine, cyclophosphamide and/or bendamustine. Lymphodepletion may be carried out for at least about one day, such as about 2 days or about 3 days.

The biological activity and/or therapeutic efficacy of the administered immune effector cell or population of immune effector cells may be measured by known methods. For example, the method may comprise imaging, such as magnetic resonance imaging.

### Antigen Binding Molecule

Also provided is an antigen binding molecule specific for one or more glioma-associated antigens. The antigen-binding molecule is selected from an antigen-binding molecule comprising the heavy chain complementarity determining regions (HCDR1, HCDR2 and HCDR3) and the light chain complementarity determining regions (LCDR1, LCDR2 and LCDR3) from an amino acid sequence selected from any one of SEQ ID NOs: 21-32 and 34-37. In some cases, the antigen-binding molecule is selected from an antigen-binding molecule comprising a polypeptide, such as one or more polypeptides, comprising HCDR1-3 and LCDR1-3 of an amino acid sequence selected from any one of SEQ ID NOs: 21-32 and 34-37 and at least 70%, at least 80%, at least 90%, at least 95%, at least 96%, at least 97%, at least 98%, at least 99% or 100% sequence identity to the amino acid sequence selected from SEQ ID NOs: 21-32 and 34-37.

The antigen-binding molecule may be an antibody. The antigen-binding molecule may be an antibody-drug conjugate. The antigen-binding molecule may be used in an antibody-based therapy, for example, in a method of metabolite radiotherapy. The antigen-binding molecule may be used in a method of treatment of a cancer as described herein.

In some cases, the antigen binding molecule comprises one or more of the antigen binding domains disclosed herein. For example, the antigen binding molecule may comprise a polypeptide disclosed herein. The antigen binding molecule may comprise the scFv or V_{HH} molecules disclosed herein. For example, the antigen binding molecule may comprise HCDRs1-3 and LCDRs1-3 of an scFv disclosed herein. The antigen binding molecule may comprise CDRs1-3 of a V_{HH} disclosed herein.

In some cases, the antigen-binding molecule comprises an immunoglobulin variable domain, such as a VHH and comprises CDRs1-3 of an amino acid sequence selected from any one of SEQ ID NOs: 83-93. The antigen-binding molecule may further comprise at least 70%, at least 80%, at least 90%, at least 95%, at least 96%, at least 97%, at least 98%, at least 99% or 100% sequence identity to an amino acid sequence selected from SEQ ID NOs: 83-93.

In some cases, the antigen-binding molecule comprises a polypeptide having at least two immunoglobulin variable domains, such as an scFv. One variable domain is typically a V_{H} and one variable domain is typically a V_{L}. The variable domains may be an scFv or an antibody. The VH may comprise HCDRs 1-3 and the VL may comprise LCDRs 1-3 of an amino acid sequence selected from any one of SEQ ID NOs: 21-32 and 34-37. The VH domain may further comprise at least 70%, at least 80%, at least 90%, at least 95%, at least 96%, at least 97%, at least 98%, at least 99% or 100% sequence identity to the V_{H} domain of the amino acid sequence selected from SEQ ID NOs: 21-32 and 34-37. The V_{L} domain may further comprise at least 70%, at least 80%, at least 90%, at least 95%, at least 96%, at least 97%, at least 98%, at least 99% or 100% sequence identity to the V_{L} domain of the amino acid sequence selected from SEQ ID NOs: 21-32 and 34-37.

### Definitions

It is to be understood that different applications of the disclosed CARs, cells, or pharmaceutical compositions of the invention may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural references unless the content clearly dictates otherwise. Thus, for example, reference to “a CAR” includes two or more “CARs”.

It is to be understood that the term “nucleic acid” and the term “polynucleotide” are used interchangeably herein.

For the purpose of this invention, in order to determine the percent identity of two sequences (such as two nucleic acids or two nucleic acids sequences), the sequences are aligned for optimal comparison purposes (e.g. gaps can be introduced in a first sequence for optimal alignment with a second sequence). The nucleotide or amino acid residues at each position are then compared. When a position in the first sequence is occupied by the same nucleotide or amino acid as the corresponding position in the second sequence, then the nucleotides or amino acids are identical at that position. The percent identity between the two sequences is a function of the number of identical positions shared by the sequences (i.e., % identity=number of identical positions/total number of positions in the reference sequence ×100).

Typically, the sequence comparison is carried out over the length of the reference sequence. For example, if the user wished to determine whether a given (“test”) sequence is 95% identical to SEQ ID NO: 1, SEQ ID NO: 1 would be the reference sequence. To assess whether a sequence is at least 95% identical to SEQ ID NO: 1 (an example of a reference sequence), the skilled person would carry out an alignment over the length of SEQ ID NO: 1, and identify how many positions in the test sequence were identical to those of SEQ ID NO: 1. If at least 95% of the positions are identical, the test sequence is at least 95% identical to SEQ ID NO: 1. If the sequence is shorter than SEQ ID NO: 1, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

By “specific” or “specifically binds”, it is meant that the antigen-binding region of a CAR binds to one or more antigenic determinants of the desired glioma-associated antigen and does not bind to other polypeptides. For example, a CAR specific for PTPRZ1 binds to an antigen of PTPRZ1 but does not bind to an antigen of a different polypeptide such as bovine serum albumin. A CAR may specifically bind if it binds to the desired glioma-associated antigen with a stronger affinity when compared to binding an antigen of a different polypeptide such as bovine serum albumin. Methods for measuring the affinity of binding are well known in the art.

As used herein, the term “about” may be interpreted to mean a value within +/−10% of the recited value.

All publications, patents and patent applications cited herein, whether supra or infra, are hereby incorporated by reference in their entirety.

The following examples illustrate the invention.

### Examples

### Example 1-Generation of Anti-PTPRZ1 RNA CAR T Cells

Six different single-chain variable fragments (scFv) against PTPRZ1 were obtained from screening a human scFvs phage display library. All scFvs were fused to the hinge-transmembrane domain of the human CD8α molecule followed by the intracellular domain of human 4-1BB (CD137) and the human CD3-2 (aka BBz CARs) or to the human IgG4 molecule hinge follow by transmembrane and intracellular domain of human CD28 and CD3-5 (aka 28z CARs). All CARs constructs were cloned in a pDA plasmid designed to optimally produce mRNA molecules under the T7 viral promoter.

Human T cells were purified from healthy donor blood with a RosetteSep cocktail (StemCell Technologies). T cells were activated during 48 h with anti-CD3/anti-CD28 Dynabeads (Invitrogen) in 1:1 cell to bead ratio. Beads were then removed from the activated T cells and the mRNA of different CAR molecules was electroporated using a MaxCyte device. After overnight recovery of the CAR T cells in presence of 30 IU/mL of IL-2, cells were frozen.

To evaluate CAR T cell killing capacity, tumor target cells were stained with a cell tracker and seeded in a 96-well plate, 25000 cells/well. CAR T cells were thawed the day before the experiment, recovered overnight in presence of 45 IU/mL of IL-2, and added to tumor cells at different effectors to target (E:T) ratios. Before adding CAR T cells, expression of the CAR molecule on the surface of T cells was measured by flow cytometry.

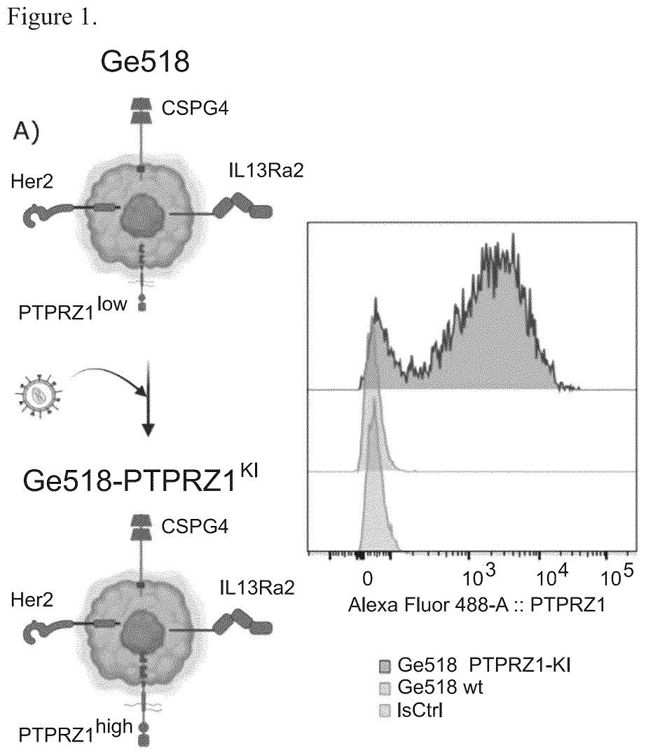

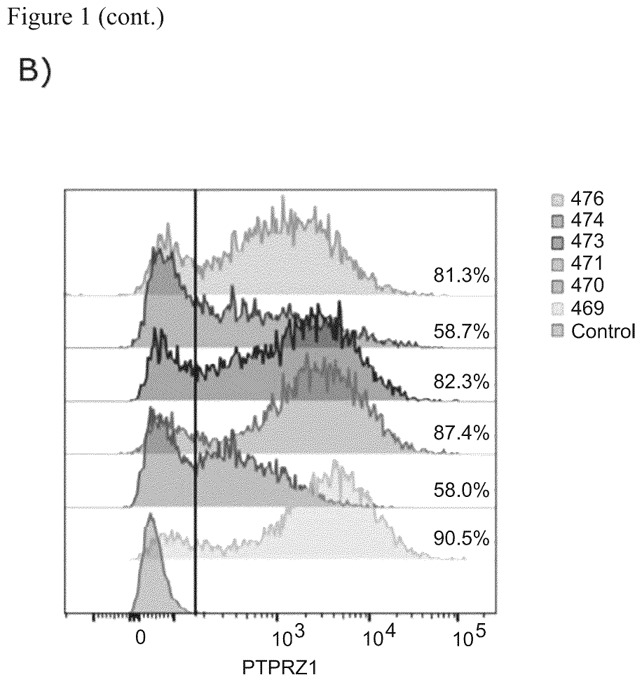

The GBM cell line Ge518, obtained in our lab from a patient tumor resection, was used as a tumor model to evaluate CAR T cell efficacy. As PTPRZ1 expression was low in these cells, the antigen was overexpressed by introducing (knock-in) an extracellular portion (domains 1 plus 2) of the human PTPRZ1 molecule under the EF1a promoter, using a 3^{rd} generation LV vector with puromycin selection. The new cell line, Ge518_PTPRZ1-KI showed high expression of PTPRZ1 at the cell surface as measured by flow cytometry using our scFvs conjugated to a rabbit Fc followed by an anti-rabbit IgG conjugated to AlexaFluo488 (FIG. 1A). The six scFvs generated against PTPRZ1 were able to specifically recognize PTPRZ1 on Ge518_PTPRZ1-KI cells, scFvs 469, 471, 473 and 476 showing a higher level of recognition (80% or higher with a clearly defined peak) than scFvs 470 and 474 (FIG. 1B).

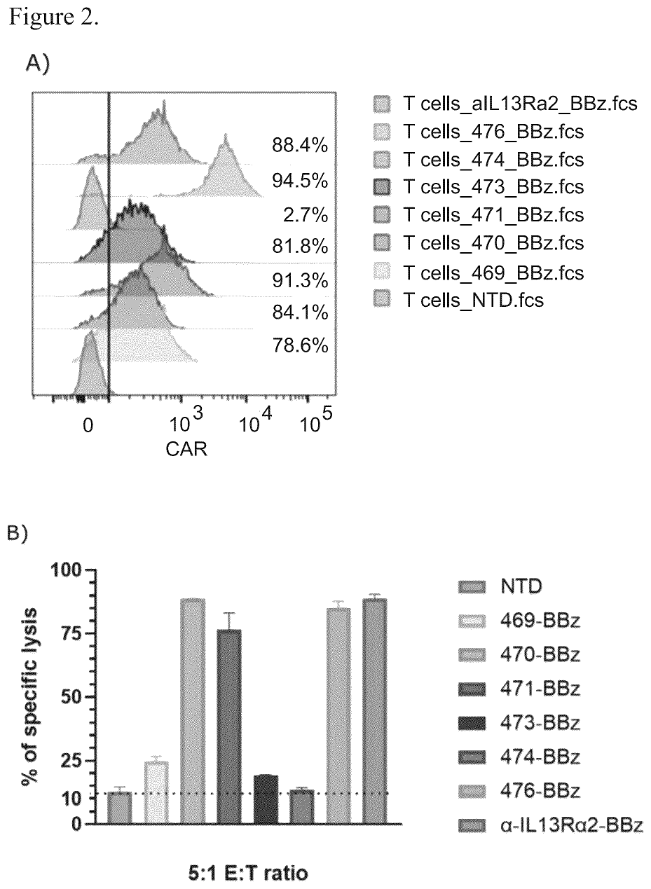

Generation of CAR T cells was efficient with, in general, more than 80% of T cells expressing the CAR molecule, and viability after thawing of 80 to 90% (FIG. 2A). The only exception was 474_BBz CAR T cells that showed low CAR expression. The six different CAR T cells and control non-transduced T cells (NTD) were incubated for 72h with Ge518_PTPRZ1-KI cells in an E:T ratio of 5:1. Positive control CAR T cells specific for IL13Rα2 were also used. Three of the anti-PTPRZ1 CAR T cells, 470_BBz, 471_BBz and 476_BBz, were capable to kill most tumor cells (75-85%, FIG. 2B), as measured by flow cytometry. The 469 BBz and 473 BBz CAR T cells showed a lower percentage of Ge518_PTPRZ1-KI tumor cells killed. The 474_BBz CAR was expressed at too low a level to show any killing capacity (FIG. 2B).

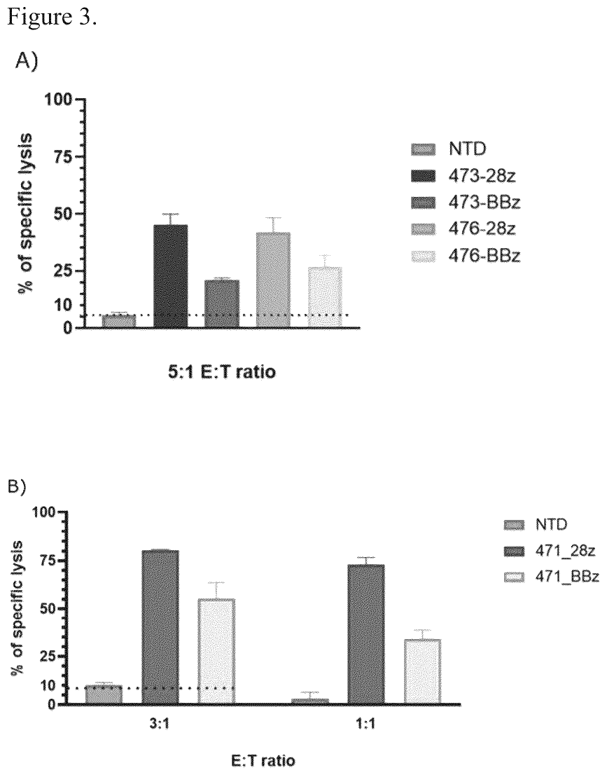

2^{nd} generation CAR T cells can use different intracellular co-stimulatory domains, 4-1BB and CD28 being the more frequently used. While 4-1BB seems to be more suitable for in vivo CAR T cell persistence, CD28 is described to provide a more potent acute response. Since RNA CAR T cells have a relatively short CAR cell surface expression (fewer than 7 days), use of co-stimulatory domains that generate a strong immune response could be desirable. The killing efficacy of CAR T cells bearing the same scFv but incorporating the 4-1BB (BBz) or CD28 (28z) domains was therefore evaluated. Both CAR T cells versions containing scFvs 473 and 476 in an E:T ratio of 5:1 were incubated with the Ge518_PTPRZ1-KI cells. After 72 h, the killing capacity of CAR T cells was measured by flow cytometry. For both scFvs, the 28z variant showed higher cytotoxicity than the BBz variant (FIG. 3A). The comparison was repeated using the scFv 471 and evaluating two different E:T ratios, 3:1 and 1:1. In the two conditions, the 471 28z variant showed a higher killing capacity than the 471_BBz variant, the difference being more pronounced at 1:1, at which 471_BBz showed reduced cytotoxicity (FIG. 3B).

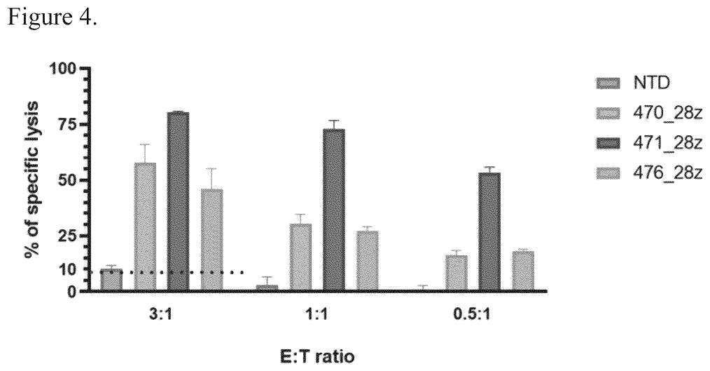

To then establish which anti-PTPRZ1 CAR T cells have the higher cytotoxic capacity, a killing assay was performed using the Ge518_PTPRZ1-KI target cells and CAR T cells 470_28z, 471_28z and 476_28z at three different E:T ratios. The comparison showed that 471_28z CAR T cells have a higher killing capacity at any of the E:T ratios tested, with a maximum of ~80% at 3:1 E:T ratio as compared to 45-60% for the other two (FIG. 4). In addition, 471_28z CAR T cells were able to keep this activity at a relatively low 0.5:1 E:T ratio, whereas the others showed low killing (<20%) (FIG. 4).

### Example 2-Anti-CSPG4 RNA CAR T Cells

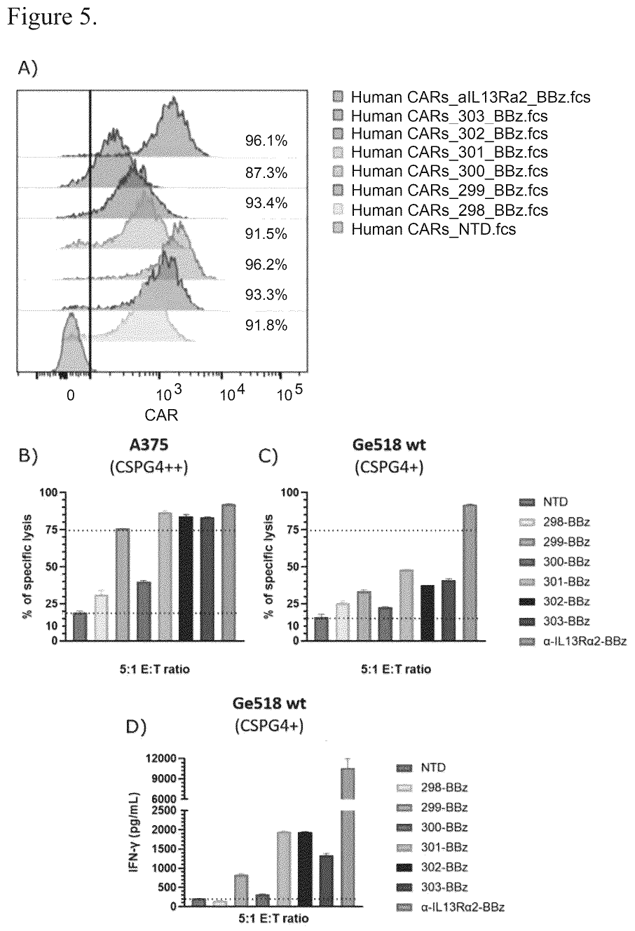

Six different scFvs against CSPG4 were obtained from screening of a human scFvs phage display library. These scFvs were cloned in CAR BBz and 28z formats in the pDA plasmid. The A375 melanoma cell line and the GBM Ge518 cell line were used as tumor models to evaluate the cytotoxic activity of anti-CSPG4 CAR T cells. RNA CAR T cells were generated following the protocol described in Example 1. All of the anti-CSPG4 CAR T cells have a high expression of the CAR molecule on their surface (>87%) (FIG. 5A).

The anti-CSPG4_BBz CAR T cells were incubated with the highly CSPG4-expressing A375 cell line for 72 h at an E:T ratio of 5:1. Anti-IL13Rα2 BBz CAR T cells were used as a positive control and non-transduced T cell as negative control, all being generated from the same donor. Four of the CSPG4-specific CAR T cells, incorporating the 299 BBz, 301_BBz, 302_BBz and 303_BBz scFv, showed a high percentage of tumor cell killing (>75%, FIG. 5B). Next, the anti-CSPG4 CAR T cells were tested against Ge518 GBM cells, at an ET ratio of 5:1. Similarly, CAR T cells incorporating the 299 BBz, 301 BBz, 302_BBz and 303_BBz scFv showed highest killing against the GBM cell line, with 35 to 45% tumor cells death (FIG. 5C). IFN-γ secretion was also measured in the supernatant of the killing experiment and found that highest levels were produced by the 301 BBz and the 302_BBz CAR T cells (~2000 μg/mL) (FIG. 5D).

### Example 3-Activity of a Mix of Three CAR T Cells Against GBM Cells

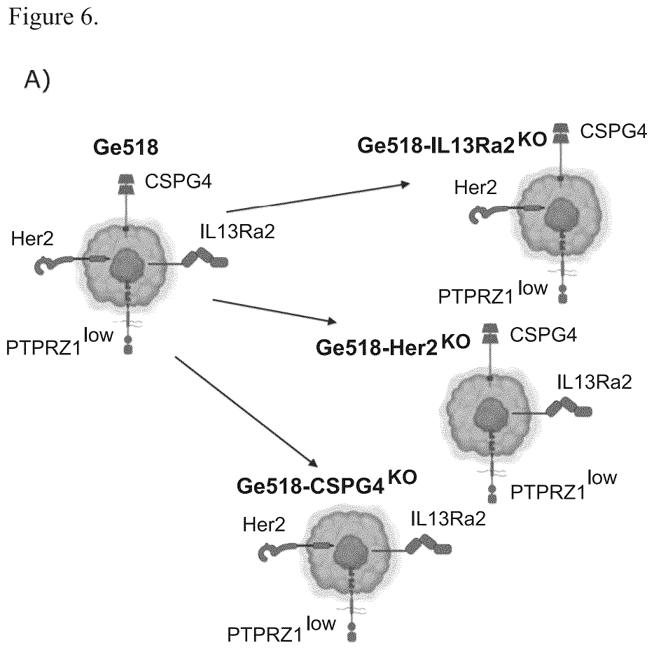

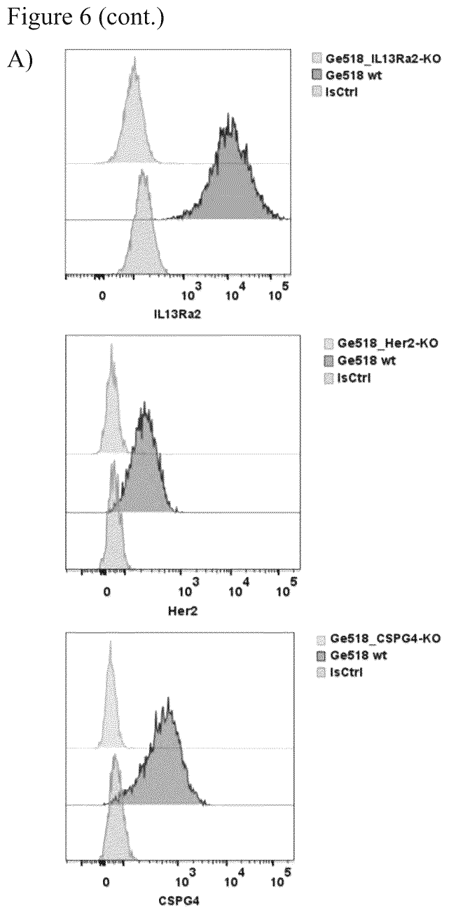

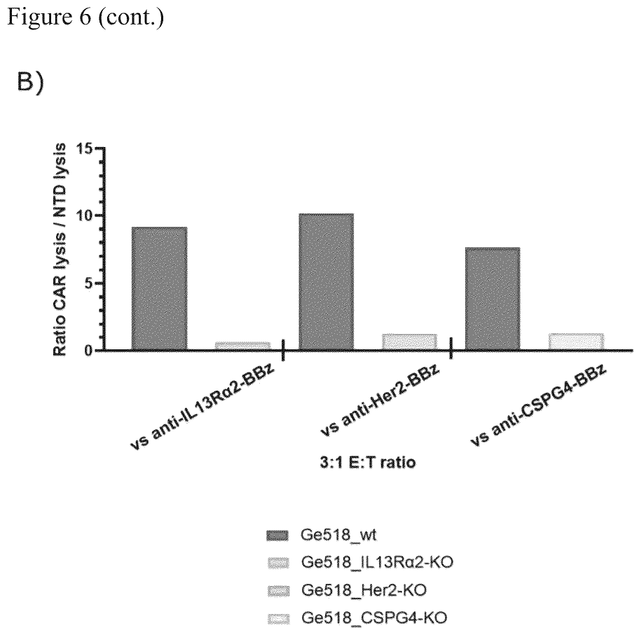

RNA CAR T cells were produced against three different GBM targets, IL13Rα2, Her2 and CSPG4 containing scFv 302. To evaluate the effect of a combined therapy against heterogeneous GBM, three variants of the Ge518 tumor cell line were generated, each lacking one of the antigens of interest. The knock-out (KO) variants were generated using the CRISPR-Cas9 system and after a cloning process the expression of the antigens was tested by flow cytometry and sequences of the genes were performed to corroborate the mutation (FIG. 6A). The differential capacity of each CAR T cell to kill the wild type (wt) variant of Ge518 in comparison to their respective antigen KO variant was evaluated by flow cytometry. In each case, the CAR T cell was able to specifically kill the wt but not the KO variant (FIG. 6B).

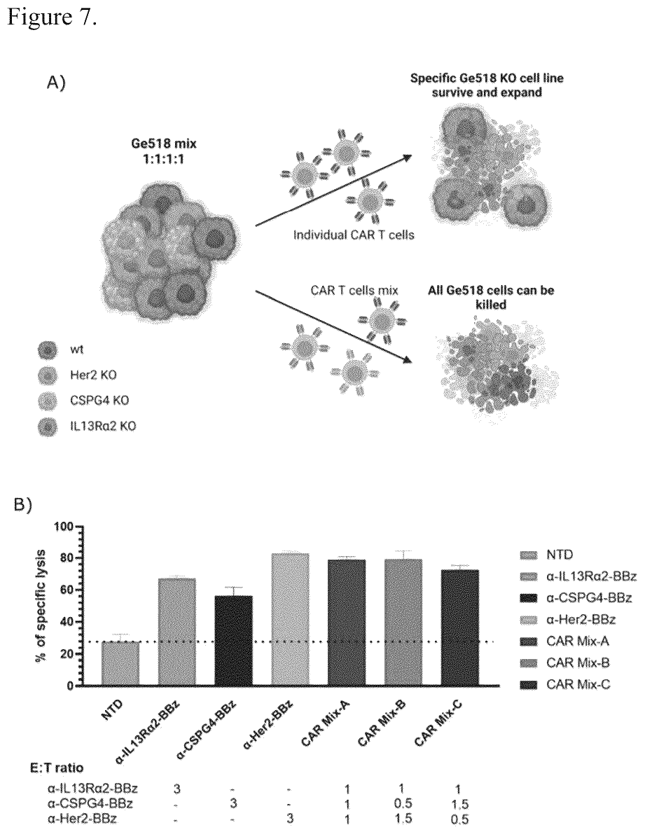

A mix of the three different Ge518 KO cells (IL13Rα2-KO, Her2-KO and CSPG4-KO) and the Ge518 wt cells were then incubated for 72 h with a mix of the anti-Her2_BBz, anti-IL13Rα2-BBz, and anti-CSPG4_BBz CAR T cells. CAR T cells were plated to obtain a final E:T ratio of 3:1, whether for individual CAR T cells and for the sum of all CAR T cells in mixes. For the mixes, equal proportions (mix A, E:T ratio of 1:1 for each CAR T cell) or two unbalanced mixes: mix B, E:T ratios of 1.5:1 of anti-Her2, 1:1 of anti-IL13Rα2 and 0.5:1 of anti-CSPG4 and mix C, E:T ratios of 0.5:1 of anti-Her2, 1:1 of anti-IL13Rα2 and 1.5:1 of anti-CSPG4 were used. The killing capacity of individual or CAR T cell mixes was measured by flow cytometry after 72 h. All mixes showed high cytotoxic activity with >70% of tumor cells death, similar to that obtained with the individual anti-Her2 BBz CAR T cell at a 3:1 E:T ratio and higher than the killing showed by individual anti-IL13Rα2 BBz or anti-CSPG4_BBz at a 3:1 E:T ratio (FIG. 7B). Mixing CAR T cells, in addition to addressing tumor heterogeneity, also lowers the possibility of tumor escape as compared to individual CAR T cells.

### Example 4-Generation of Triple CAR T Cells

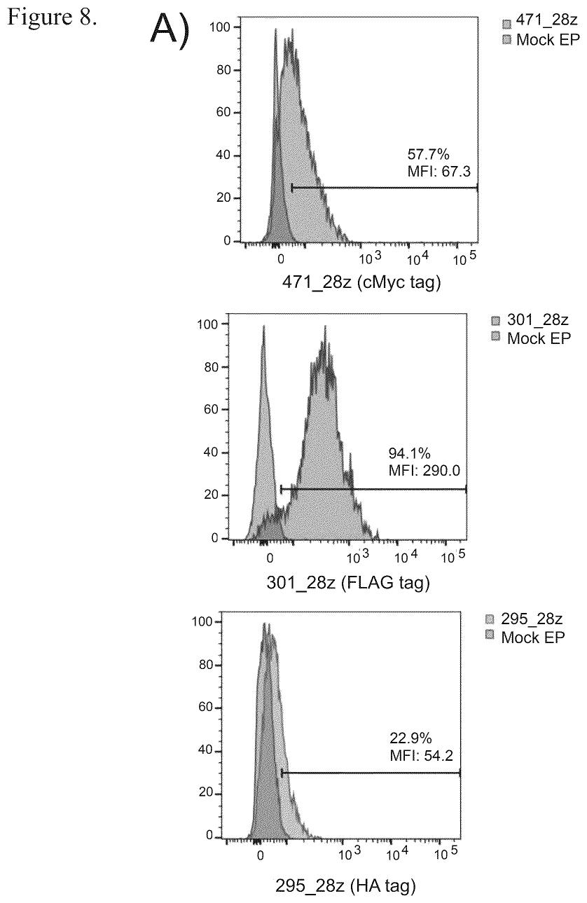

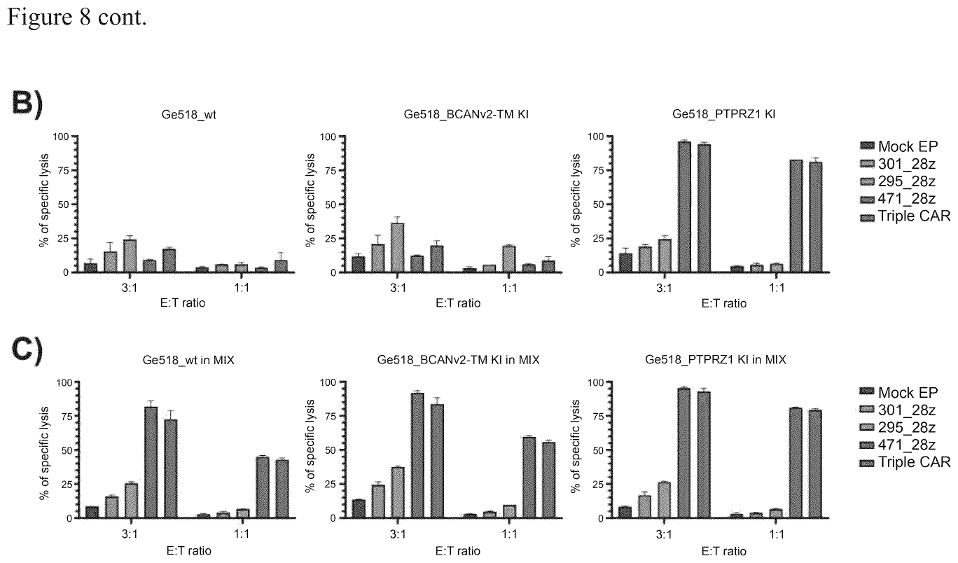

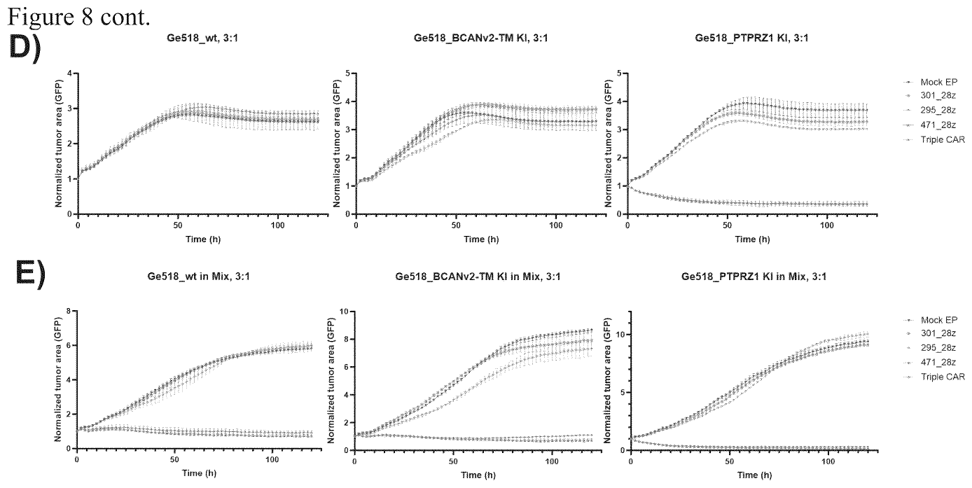

RNA CAR T cells were produced against three different glioma-associated antigens, PTPRZ1 (471_28z), CSPG4 (301_28z) and BCAN (295_28z) using scFvs specific for each antigen. Expression of each CAR individually was confirmed (FIG. 8A). The _28z constructs comprised, in addition to the scFv sequences, a human IgG4 hinge sequence (SEQ ID NO: 118), a CD28 transmembrane and intracellular domain (SEQ ID NOs: 123 and 125, respectively), and a CD3zeta intracellular domain (SEQ ID NOs: 126 to 128).

A ‘triple’ RNA CAR T cell was produced by simultaneous RNA electroporation of human T cells, generating a CAR T cell transfected with RNAs that encoded, and which expressed, all three of the anti-PTPRZ1 (471_28z), anti-CSPG4 (301_28z) and anti-BCAN (295_28z) CARs. To evaluate the effect of a combined therapy against heterogeneous GBM, two variants of the Ge518 tumor cell line were generated: Ge518 BCANv2-TM KI, a knock-in of BCAN, and Ge518_PTPRZ1 KI, a known-in of PTPRZ1. Across all three target cell-lines, the triple CAR T cell led to increased cell killing than any monovalent CAR T cell (FIG. 8B). When all three target cell lines were mixed, as is more representative of a heterogeneous GBM, enhanced cell killing of the Ge518_wt and Ge518_BCANv2 cell lines was observed, particularly by the monovalent anti-PTPRZ1 CAR T cell and the Triple CAR (FIG. 8C). This is indicative of a ‘bystander’ effect. These results were reflected in in vitro models of inhibition of tumour growth (FIGS. 8D and 8E).

### Example 5-Safety and Analysis of the Bystander Effect

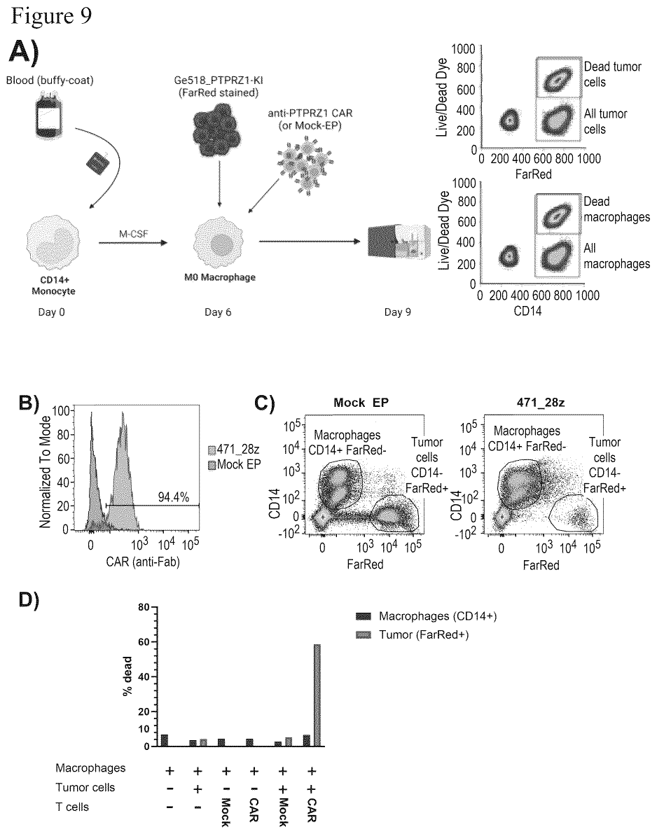

As demonstrated in Example 4, a monovalent anti-PTPRZ1 CAR T cell was discovered to efficiently kill GBM cell lines that did not significantly express PTPRZ1, when present in a mix with a GBM cell line that did express PTPRZ1. It was then studied whether this bystander effect was specific to GBM cells or also effected healthy cells, by mixing Ge518_PTPRZ1-KI cells with ‘healthy’ macrophages. Upon addition of monovalent anti-PTPRZ1 CAR T cells, cell killing of the Ge518_PTPRZ1-KI cells was observed, whereas no significantly increased killing of the macrophages was observed (FIG. 9).

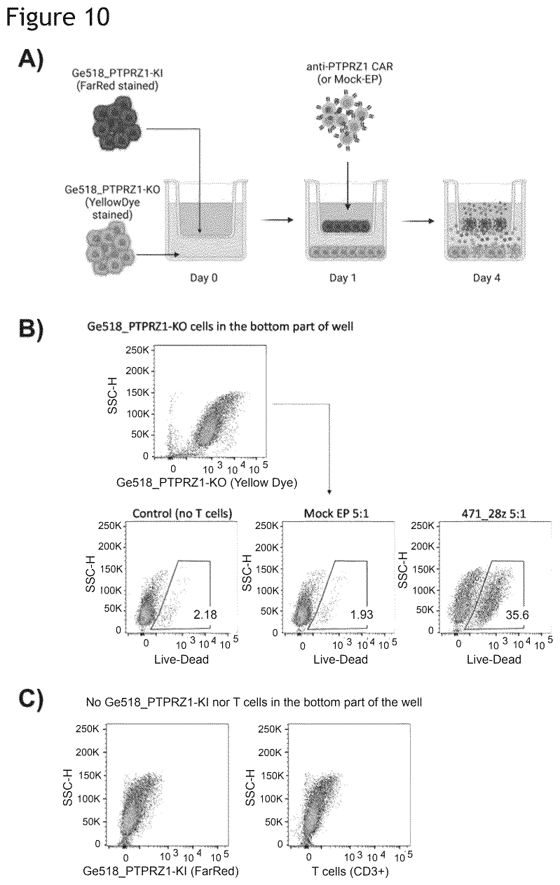

To determine whether the ‘bystander’ effect was mediated by soluble factors, an experiment was set up according to FIG. 10A. Cell killing of a GBM cell-line with PTPRZ1 knocked out (Ge518_PTPRZ1-KO) was observed when Ge518_PTPRZ1-KI cells were contacted with monovalent anti-PTPRZ1 CAR T cells, despite no direct contact between the monovalent anti-PTPRZ1 CAR T cells and the Ge518_PTPRZ1-KO cell line.

### Example 6-Co-Dependency of Target Expression in Human Glioblastoma

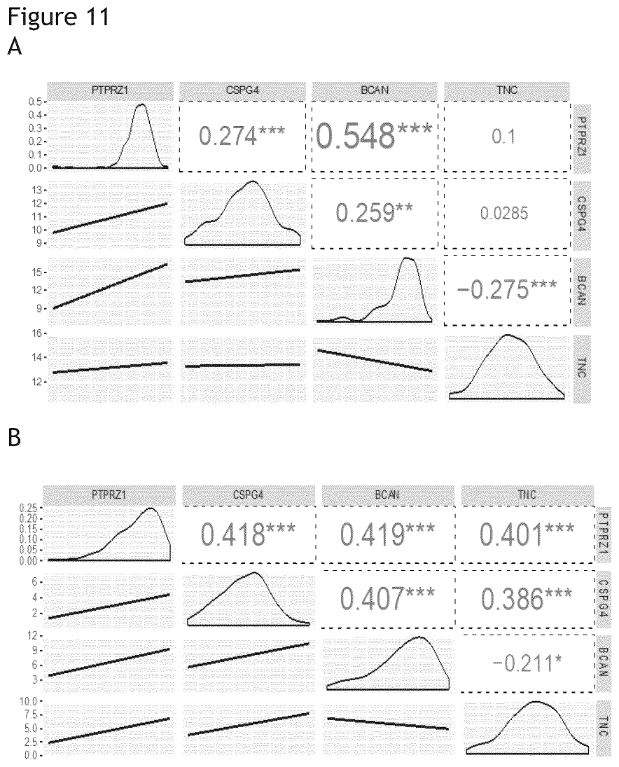

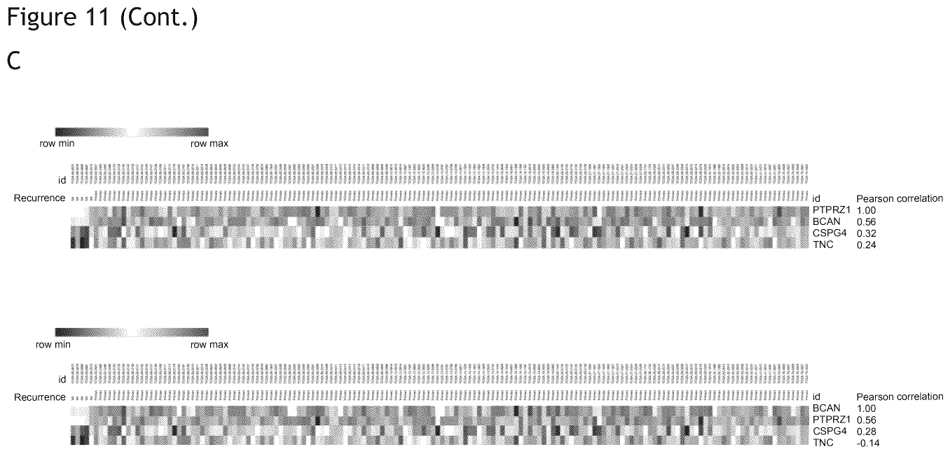

The correlation between expression of pairs of antigens was studied using bulk RNA-seq data from TCGA (primary GBM) data and from CGGA (recurrent GBM) data (FIGS. 11A and 11B).

Whilst CAR T cell therapy could be tailored to the patient depending on antigen expression, this data may also be used to find optimal combinations that take in account inter-patient variability, i.e. without knowledge of antigen expression of the glioma, we can improve our chances that a multivalent CAR T cell therapy will target the glioma by selecting combinations of antigens which poorly correlate or negatively correlate. These antigens have different trend of expression in different patients. The combination should therefore target a wide number of patients. Possible combinations could include BCAN, TNC, CSPG4.

Conversely, where expression of one or more glioma-associated antigens are known in a patient, using a multivalent CAR T cell that also targets glioma-associated antigens that are known to positively correlate with the known antigen may also be useful, for example, to reduce the chances that the glioma can evade the CAR T cell therapy by reducing expression of a single antigen.

### Example 7-Generation of Monovalent and Multivalent CAR T Cells Using Nanobodies

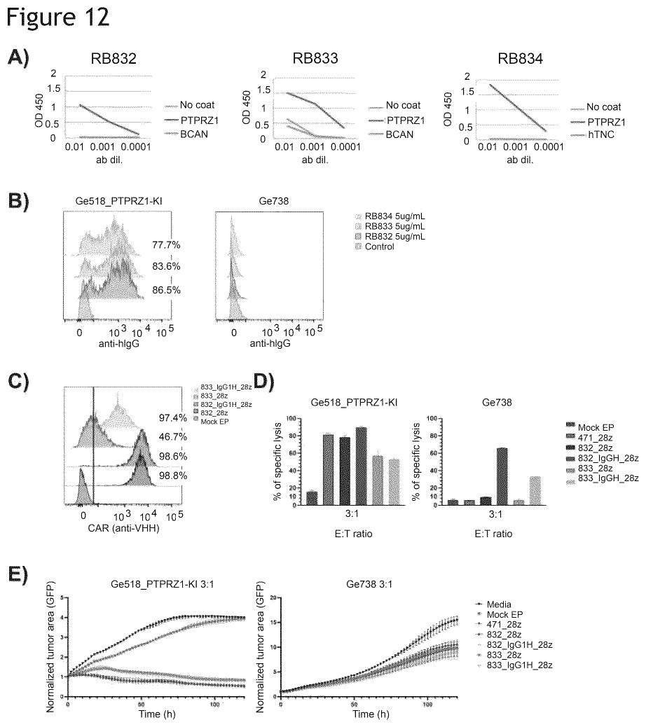

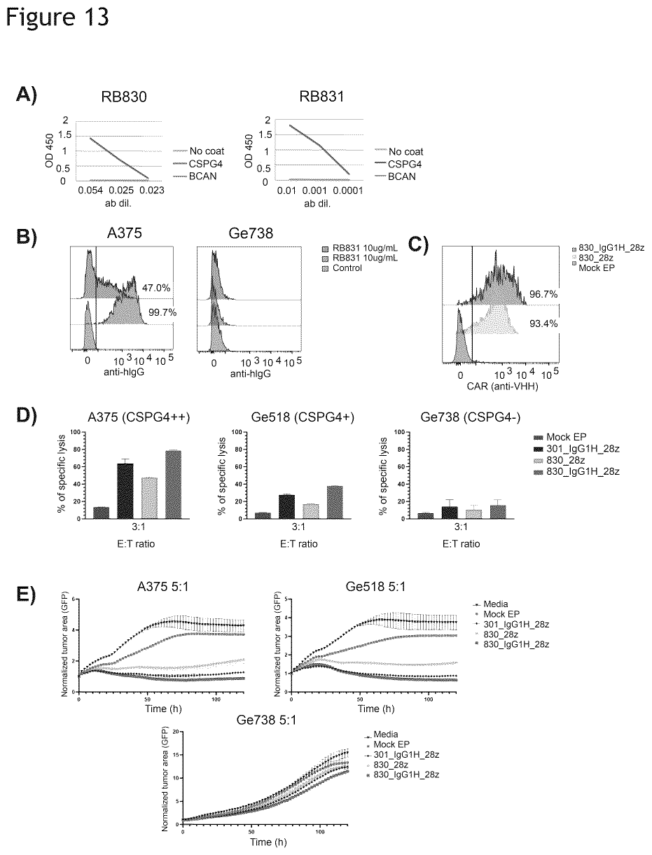

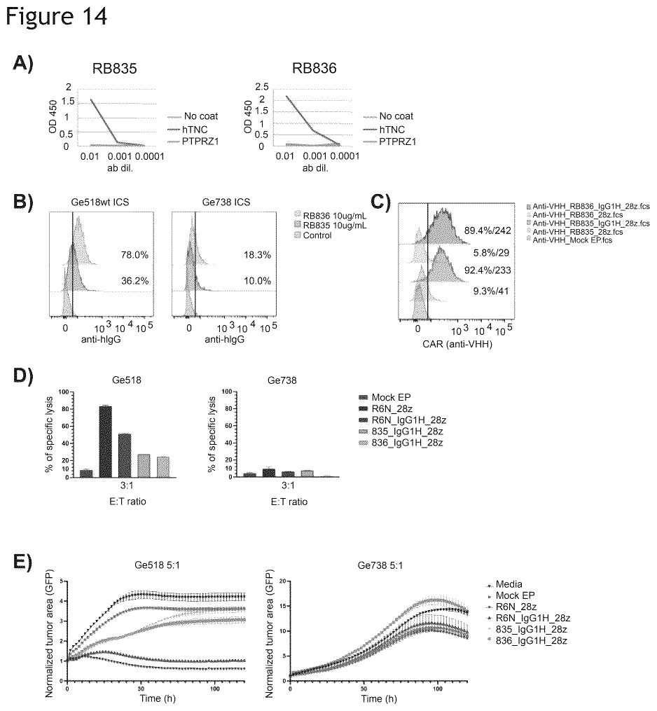

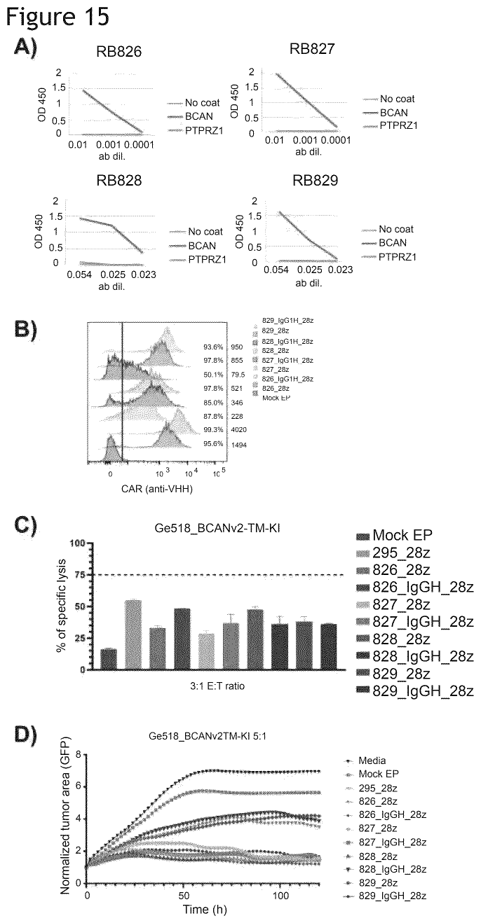

Nanobodies (VHHs) were generated against BCAN (RB826-829), CSPG4 (RB830-831), PTPRZ1 (832-834) and TNC (835-836). The specificity of the nanobodies was tested by ELISA against their target antigen and a control antigen (FIGS. 12A, 13A, 14A and 15A). The nanobodies were conjugated to human IgG1 Fc and recognition of tumour cell lines was examined (FIGS. 12B, 13B and 14B).

CARs were generated using the nanobodies with short or long hinges, and the expression of each CAR by transformed T cells was examined to determine expression levels of each construct (FIGS. 12C, 13C, 14C and 15B). The short “_28z” constructs comprised, in addition to the nanobody sequences, a human IgG4 hinge sequence (SEQ ID NO: 118), a CD28 transmembrane and intracellular domain (SEQ ID NOs: 123 and 125, respectively), and a CD3zeta intracellular domain (SEQ ID NOs: 126 to 128). The long “IgG1H_28z” constructs comprised, in addition to the nanobody sequences, a human IgG1 long hinge sequence (SEQ ID NO: 121), a CD28 transmembrane and intracellular domain (SEQ ID NOs: 123 and 125, respectively), and a CD3zeta intracellular domain (SEQ ID NOs: 126 to 128). Cell killing of the nanobody-based CAR-T cells was determined by flow cytometry against a number of tumour cell lines (FIGS. 12D, 13D, 14D and 15C). Corresponding data was generated for in vitro inhibition of tumour growth (FIGS. 12E, 13E, 14E and 15D).

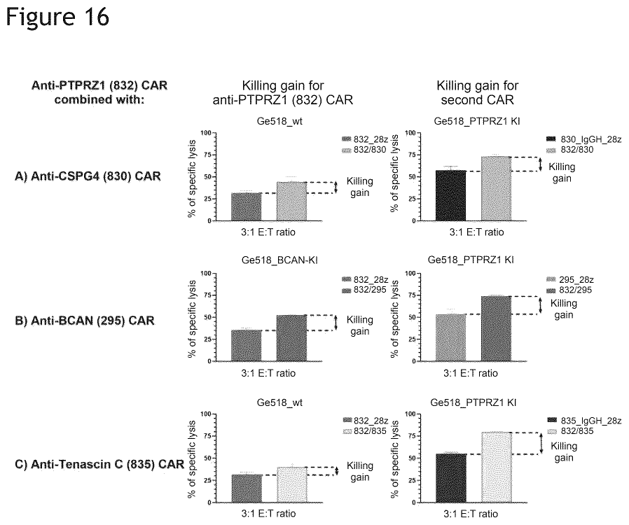

Bispecific CAR T cells were generated expressing a CAR comprising the anti-PTPRZ1 nanobody 832 in combination with a CAR comprising the anti-CSPG4 nanobody 830, the anti-BCAN scFc 295 or the anti-TNC nanobody 835. The graphs in the left column of FIG. 16A-16C compare the cell killing of a cell-line with low expression of PTPRZ1 but higher expression of CSPG4 (FIG. 16A), BCAN (FIG. 16B) or TNC (FIG. 16C) by a monovalent anti-PTPRZ1 CAR T cell versus a bispecific CAR T cell. The graphs in the right column of FIG. 16A-16C compare the cell killing of a cell-line with high expression of PTPRZ1 by a monovalent anti-CSPG4 CAR T cell (FIG. 16A), a monovalent anti-BCAN CAR T cell (FIG. 16B) or a monovalent anti-TNC CAR T cell (FIG. 16C) versus a bispecific CAR T cell also expressing an anti-PTPRZ1 CAR.

### Numbered Further Embodiments

1. An immune effector cell or a population of immune effector cells expressing one or more chimeric antigen receptors (CARs) specific for one or more glioma-associated antigens.

2. The immune effector cell or population of immune effector cells according to embodiment 1, wherein the one or more glioma-associated antigens are PTPRZ1, BCAN, CSPG4 and/or TNC.

3. The population of CAR-expressing immune effector cells according to embodiment 1 or 2 which comprises at least two different CAR-expressing immune effector cells, wherein each different CAR-expressing immune effector cell is specific for a different glioma-associated antigen, optionally wherein the population of CAR-expressing immune effector cells comprises at least three different CAR-expressing immune effector cells.

4. A method of making an immune effector cell or a population of immune effector cells according to any one of the preceding embodiments, comprising transforming said cell or said population of cells with one or more nucleic acids encoding one or more CARs specific for one or more glioma-associated antigens.

5. A method of treating cancer in a subject, comprising administering to the subject an effective amount of an immune effector cell or a population of immune effector cells according to any one of embodiments 1-3.

6. The immune effector cell or a population of immune effector cells of any one of embodiments 1-3 for use in a method of treating cancer.

7. The method of treating a subject according to embodiment 5, or the immune effector cell or population of immune effector cells for use according to embodiment 6, wherein the cancer is glioma.

8. The immune effector cell or population of immune effector cells of any one of embodiments 1-3, the method of any one of embodiments 4, 5, or 7, or the immune effector cell or the population of immune effector cells for use according to embodiment 6 or 7, wherein the one or more CARs are selected from CARs comprising a polypeptide having the amino acid sequence of any one of SEQ ID NOs: 21-40 or an amino acid sequence with at least 80%, 85%, 90%, 95% or 99% identity thereto.

9. The immune effector cell or population of immune effector cells of any one of embodiments 1-3 or 8, the method of any one of embodiments 4, 5, 7 or 8, or the immune effector cell or the population of immune effector cells for use according to any one of embodiments 6 to 8, wherein the one or more CARs are selected from CARs comprising a polypeptide comprising the heavy chain complementarity determining regions (HCDR1, HCDR2 and HCDR3) and the light chain complementarity determining regions (LCDR1, LCDR2 and LCDR3) from an amino acid sequence selected from any one of SEQ ID NOs: 21-40.

10. The immune effector cell or population of immune effector cells of any one of embodiments 1-3, 8 or 9, the method of any one of embodiments 4-5 or 7-9, or the immune effector cell or the population of immune effector cells for use according to any one of embodiments 6 to 9, wherein the cell or cells are T cells, NK cells, iPSC-NK cells, γδ T cells, phagocytes or macrophages.

11. A CAR comprising a polypeptide having (a) the amino acid sequence of any one of SEQ ID NOs: 21-40, (b) an amino acid sequence with at least 80%, 85%, 90%, 95% or 99% identity thereto, and an intracellular signalling domain, or (c) the heavy chain complementarity determining regions (HCDR1, HCDR2 and HCDR3) and the light chain complementarity determining regions (LCDR1, LCDR2 and LCDR3) from an amino acid sequence selected from any one of SEQ ID NOs: 21-40.

12. A multivalent CAR comprising (a) an extracellular domain specific for two or more glioma-associated antigens, optionally wherein the glioma-associated antigens are selected from PTPRZ1, BCAN, CSPG4 and/or TNC; and (b) and an intracellular signalling domain.

13. The multivalent CAR of embodiment 12, wherein the extracellular domain is an ScFv, VH or VHH.

14. The multivalent CAR of embodiment 13, wherein the extracellular domain comprises polypeptides selected from (a) the amino acid sequences of SEQ ID NOs: 21-40, (b) amino acid sequences with at least 80%, 85%, 90%, 95% or 99% identity thereto, or (c) amino acid sequences comprising the heavy chain complementarity determining regions (HCDR1, HCDR2 and HCDR3) and the light chain complementarity determining regions (LCDR1, LCDR2 and LCDR3) from an amino acid sequence selected from SEQ ID NOs: 21-40.

15. The CAR of embodiment 11 or the multivalent CAR of any one of embodiments 12 to 14, wherein the intracellular domain comprises a CD3 zeta signalling domain alone or in combination with a CD28, CD27, CD134 (OX40), and/or CD137 (4-1BB) intracellular domain.

16. The CAR of embodiment 11 or 15, or the multivalent CAR of any of embodiment 12 to 15, further comprising a transmembrane domain.

17. The CAR or the multivalent CAR of embodiment 16, wherein the transmembrane domain is a CD28 transmembrane domain.

18. A nucleic acid encoding the CAR of any one of embodiments 11 and 15 to 17, or the multivalent CAR of any one of embodiments 12 to 17.

19. The nucleic acid of embodiment 18, which is a DNA or RNA.

20. The nucleic acid of embodiment 18 or 19, comprising the sequence of any one of SEQ ID NOs: 1-20 or a sequence encoding the amino acid sequence of any one of SEQ ID NOs: 21-40.

21. A vector comprising one or more nucleic acids according to any one of embodiments 18 to 20.

22. The vector of embodiment 21 which is a lentiviral vector, an RNA vector, liposomes or lipid nanoparticles for RNA in vivo delivery.

23. An antigen binding molecule specific for one or more glioma-associated antigens, selected from an antigen-binding molecule comprising (a) a polypeptide having the amino 5 acid sequence of any one of SEQ ID NOs: 21-32 and 34-37, (b) an amino acid sequence with at least 80%, 85%, 90%, 95% or 99% identity thereto, or (c) the HCDR1, HCDR2 and HCDR3 and the LCDR1, LCDR2 and LCDR3 from an amino acid sequence selected from any one of SEQ ID NOs: 21-32 and 34-37.

1. An immune effector cell or a population of immune effector cells expressing:
(a) one or more chimeric antigen receptors (CARs) specific for two or more glioma-associated antigens, wherein one or more of the glioma-associated antigens are selected from PTPRZ1, BCAN, CSPG4 and TNC;
(b) a chimeric antigen receptor (CAR) specific for PTPRZ1; or
(c) a chimeric antigen receptor (CAR) specific for BCAN.

2. The immune effector cell or population of immune effector cells according to claim 1, wherein two or more of the glioma-associated antigens are selected from PTPRZ1, BCAN, CSPG4 and TNC.

3. The immune effector cell or population of immune effector cells according to claim 1, which expresses one or more CARs specific for three or more glioma-associated antigens.

4. The immune effector cell or population of immune effector cells according to claim 1, wherein the cell or each cell expresses two or more CARs specific for different glioma-associated antigens, or three or more CARs specific for different glioma-associated antigens.

5. (canceled)

6. The population according to claim 1, which comprises:
(i) at least two different CAR-expressing immune effector cells, wherein each different CAR-expressing immune effector cell is specific for a different glioma-associated antigen; or
(ii) at least three different CAR-expressing immune effector cells, wherein each different CAR-expressing immune effector cell is specific for a different glioma-associated antigen.

7. (canceled)

8. The immune effector cell or population of immune effector cells according to claim 1, wherein one or more of the glioma-associated antigens is a cell-surface marker and one or more of the glioma-associated antigens is an extracellular matrix (ECM) marker, optionally wherein (i) the cell-surface marker is selected from PTPRZ1, CSPG4 and BCAN, (ii) the ECM marker is selected from TNC and BCAN, or (iii) a combination of (i) and (ii).

9. (canceled)

10. (canceled)

11. (canceled)

12. A method of making an immune effector cell or a population of immune effector cells according to claim 1, comprising transforming said cell or said population of cells with one or more nucleic acids encoding one or more CARs specific for one or more glioma-associated antigens.

13. A method of treating cancer in a subject, comprising administering to the subject an effective amount of an immune effector cell or a population of immune effector cells according to claim 1, optionally wherein the cancer is glioma.

14. (canceled)

15. (canceled)

16. The immune effector cell or population of immune effector cells of claim 1, wherein the cell or cells are T cells, NK cells, iPSC-NK cells, γδ T cells, phagocytes or macrophages.

17. A CAR that is:
(i) specific for PTPRZ1;
(ii) specific for BCAN;
(iii) specific for a glioma associated antigen selected from PTPRZ1, BCAN, CSPG4 and TNC, comprising a polypeptide comprising:
(a) the heavy chain complementarity determining regions (HCDR1, HCDR2 and HCDR3) and the light chain complementarity determining regions (LCDR1, LCDR2 and LCDR3) from an amino acid sequence selected from any one of SEQ ID NOs: 21-40; or
(b) the complementary determining regions (CDR1, CDR2 and CDR3) from an amino acid sequence selected from any one of SEQ ID NOs: 83-93; or
(iv) multivalent and which comprises (a) an extracellular domain specific for two or more glioma-associated antigens, wherein one or more of the glioma-associated antigens are selected from PTPRZ1, BCAN, CSPG4 and TNC; and (b) and an intracellular signalling domain.

18. (canceled)

19. (canceled)

20. The CAR according to claim 17, wherein the polypeptide comprises:
(a) the amino acid sequence of any one of SEQ ID NOs: 21-40 and 83 to 93, and/or
(b) an amino acid sequence having at least 80%, 85%, 90%, 95% or 99% identity to the amino acid sequence of any one of SEQ ID NOs: 21-40 and 83-93.

21. The CAR according to claim 17, which further comprises an intracellular signalling domain.

22. The CAR according to claim 17, wherein the polypeptide comprises:
(a) one or more immunoglobulin variable domains; and/or
(b) an scFv, VH or VHH domain.

23. (canceled)

24. The multivalent CAR of claim 17, wherein the extracellular domain:
(i) is an ScFv, VH or VHH;
(ii) comprises a polypeptide comprising:
(a) the heavy chain complementarity determining regions (HCDR1, HCDR2 and HCDR3) and the light chain complementarity determining regions (LCDR1, LCDR2 and LCDR3) from an amino acid sequence selected from any one of SEQ ID NOs: 21-40; or
(b) the complementary determining regions (CDR1, CDR2 and CDR3) from an amino acid sequence selected from any one of SEQ ID NOs: 83-93; or
(iii) comprises:
(a) the amino acid sequence of any one of SEQ ID NOs: 21-40 and 83 to 93, or
(b) an amino acid sequence having at least 80%, 85%, 90%, 95% or 99% identity to the amino acid sequence of any one of SEQ ID NOs: 21-40 and 83-93.

25. (canceled)

26. (canceled)

27. The CAR of claim 17, wherein the CAR comprises an intracellular domain comprising a CD3 zeta signalling domain alone or in combination with a CD28, CD27, CD134 (OX40), and/or CD137 (4-1BB) intracellular domain.

28. The CAR of claim 17, further comprising a transmembrane domain, optionally wherein the transmembrane domain is a CD28 transmembrane domain.)

29. (canceled)

30. The immune effector cell or population of immune effector cells of claim 1, wherein the one or more CARs comprise a CAR or a multivalent CAR according to claim 17.

31. A nucleic acid encoding the CAR of claim 17, optionally wherein:
(a) the nucleic acid is a DNA or RNA; and/or
(b) the nucleic acid comprises the sequence of any one of SEQ ID NOs: 1-20 and 94 to 104 or a sequence encoding the amino acid sequence of any one of SEQ ID NOs: 21-40 and 83 to 93.

32. (canceled)

33. (canceled)

34. A vector comprising one or more nucleic acids according to claim 31; optionally wherein the vector is a lentiviral vector, an RNA vector, liposomes or lipid nanoparticles for RNA in vivo delivery.

35. (canceled)

36. An antigen binding molecule specific for one or more glioma-associated antigens, which comprises a polypeptide comprising the complementary determining regions (CDR1, CDR2 and CDR3) from an amino acid sequence selected from any one of SEQ ID NOs: 83-93; optionally wherein the polypeptide comprises an amino acid sequence having at least 80%, 85%, 90%, 95% or 99% identity to the amino acid sequence of any one of SEQ ID NOs: 21-40 and 83-93.

37. (canceled)


SEQUENCE LISTING (139 sequences)

SEQ ID NO: 1 - DNA, 765 bp, synthetic construct
  source 1..765: mol_type = other DNA
atggcccaggtgcaactggtggaatctgggggaggcgtggttcagcctgggaggtccctgcggctctcctgtgcagcctcaggatttaccttcagtagctacgccatgcactgggtccgccaggctccaggcaaagggttggaatgggttgcagttatatcatatgatggaagtaataaatactacgcagactccgtgaagggccgattcaccatttcacgtgacaattccaagaacacgctttatctgcaaatgaacagcttgagagctgaagatacggctgtgtattactgcgcgaggggtagtggatacagctatggtccgggttatgatgcatttgatatttggggccagggaacccttgtcacagtctcaagcggtggaggcggttcaggcggaggtggcagcggcgggggcggatctacaaattttatgctgactcagcctcattctgtatcggagtctccagggaagacagtaaccatctcctgcacacgcagcagtggcagcatcgccagcaactatgtgcagtggtaccagcagagaccaggcagttcacccactactgtgatttatgaggataaccaaagaccctctggggtccctgatcggttttctggctccatcgacagcagttccaattcggcctccctcaccatctctggactaaaaactgaggacgaggctgactactactgtcagtcctgggaccccgtgttcggggtgttcggcggagggacaaagctgaccgtcttaggggcggcc

SEQ ID NO: 2 - DNA, 762 bp, synthetic construct
  source 1..762: mol_type = other DNA
atggcacaagtgcagttagttcagtctggggctgaagtgaagaagcctgggtcctcggtgaaggtctcctgcaaggctagtggaggcaccttcagcagctatgctatcagctgggtgcgacaggcccctggacaaggccttgaatggatgggagggatcattccgatctttggtacagcaaactacgcacagaagttccaaggcagagtcactattaccgcggacgaatccacaagcacagcatacatggagctgagcagcctgaggtctgaagatacggccgtgtattactgtgcgagagaggggggggccgtggggtactactacggtatggacgtctggggccagggaacacttgtgacagtctccagcggtggaggcggttcaggcggaggcggctcaggcggtggcggatctactcagagtgccttgactcagccagcctccgtgtctgggtcacccggacagtcgataaccatctcctgcactggaaccagcagtgacgttggtggttataattatgtatcctggtaccaacagcacccaggcaaagcccccaaactcatgatttatgaggtcagtaatcggccctcaggggtttctaatcgtttctctggctccaaatctggcaacacggcctccctgactatatcagggctccaggctgaggacgaggctgattattactgtagttcatatgataggagcaaccgcagtatggtgtttggcggagggaccaaactgaccgtactaggggcagcc

SEQ ID NO: 3 - DNA, 759 bp, synthetic construct
  source 1..759: mol_type = other DNA
atggccgaagtgcagctggtggagtctgggggaggcttggtacagcctggggggtccctgcgtctctcctgtgcagcctctggatttaccttcagtagctatagcatgaactgggtcaggcaggctccagggaaggggcttgagtgggtttcatacattagtagcagtagtagcacaatatactacgcagactctgtgaagggccgattcacaatctccagggataatgccaagaactcactgtatttacaaatgaatagccttagagccgaagacacggctgtgtattactgtgcgagaccaggctacggtgactttcccggtgcttttgatatctggggccagggaaccctggtcacagtgtcgagcggtggaggcggctcaggcggaggtggcagcggcggtggcggatctacgcagtctgcattgactcagcctgcctccgtgtctggatcacctggacaaagcattaccatctcctgcactggaaccagcagtgacgttggtggttataactatgtctcctggtaccaacagcacccgggcaaagcccccaaactcatgatttacgaagtaagtaatcggccctcaggggtttctaatcgcttctctggctccaaatccggcaacactgcctccctgacaatcagtgggctccaggctgaggacgaggctgattattactgcagctcatatgattgggccacctacgggtcggtgttcggcggagggaccaagctgactgtcctaggggcggca

SEQ ID NO: 4 - DNA, 753 bp, synthetic construct
  source 1..753: mol_type = other DNA
atggcccaagtgcagctggtggagtctgggggaggcgttgtccagcctgggaggtcactgagactctcctgtgcagcctctgggtttacattcagtagctatgctatgcactgggtacgccaagctcctggcaaagggctggagtgggtggcagttatatcatatgatggtagtaacaaatactacgcagacagtgtgaagggccgattcaccatctccagagacaattccaagaacactctttatctgcaaatgaacagcctgcgggctgaagacacagctgtgtactactgtgcgcgggaccaggatgactccagtgatgcttttgatatctggggccagggaaccctggtcacagtctcgagcggtggaggcggtagcggcggaggtggcagcggcggtggcggatcgacgcagagcgttttgacgcaaccgccctcagtgtctgcagccccaggacagaaagtcaccatatcctgctctggaagcagctccaacattgggaacaattatgtatcctggtaccagcagttgccagggacagcccccaaactcctcatttacgacaataataagcgtccctcagggattcctgaccgcttttctggcagtaagtctggcacttcagccactctgggcatcaccggacttcagactggggacgaagccgattattactgcggaacatatgattacatcgcgaccagggccgtgttcggtggcgggaccaagttaactgtgctaggggcagcc

SEQ ID NO: 5 - DNA, 768 bp, synthetic construct
  source 1..768: mol_type = other DNA
atggcccaggtgcagcttgttcagtctggggctgaggtgaagaagccagggtcctcggtgaaggtttcctgcaaggcttcaggaggcaccttcagcagctatgctatcagttgggtgcggcaagcacctggccaagggcttgagtggatgggagggataatccctatctttggtacagcaaactacgcacaaaagttccagggccgcgtcacgattaccgccgacgaatccaccagcacagcctacatggaactgtccagcctgagaagtgaagacactgccgtgtattactgtgcgcgtgggacgtattacgatttttggagtggttattatgatgcttttgatatctggggccagggaaccctggttacagtctctagcggtggaggcggttcaggcggaggtggcagcggcggtggcggctctactcagtctgtgttgacacagccgccctcagtgtctgcagccccaggacagaaggtcaccatctcctgttctggtagcagctccaacattgggaataattacgtatcctggtaccagcagttgccaggaacagcccccaaactcttaatatatgacaataacaaaaggccctcagggattcctgaccgattcagtggctccaaatctggcacttcagctaccctgggcattaccggactccagactggggacgaggccgattattactgcggaacatgggatagttcgtactggcaacccgtattcggcggagggactaaactgaccgtcctaggggcggcc

SEQ ID NO: 6 - DNA, 753 bp, synthetic construct
  source 1..753: mol_type = other DNA
atggcccaggtgcaactggtggaatctgggggaggcgtggtccagcctgggaggtcgttgcggctctcctgtgcagcctctggattcacctttagtagctatgctatgcactgggtccgccaagctccaggcaaggggctggaatgggtggcagttatatcatacgatggaagtaataaatactacgcagactccgtgaagggccgcttcaccatctccagagacaattccaagaacacactgtatctgcaaatgaacagcctgagggctgaggacactgctgtgtattactgtgcgagagaccaggatgactccagtgatgcttttgatatctgggggcagggaaccctggtaacagtcagtagcggtggaggcggttcaggcggcggtggcagcggcggtggcggtagcacgcagtctgtgttgacgcagccgccctcagtgtctgcagccccaggacaaaaggtcactatctcctgctctggaagcagctccaacattgggaacaactatgtatcctggtaccagcagttacctggtacagcccccaaactcctcatttatgacaataataagcgaccctcagggattcctgaccgttttagtggcagcaaatctggcacttcagccacccttggcatcaccggactccagactggggacgaggccgattattactgcggaacatgggattacaaagtttcgcggcttgtcttcggcggagggaccaagctgacagttctaggggcggcc

SEQ ID NO: 7 - DNA, 753 bp, synthetic construct
  source 1..753: mol_type = other DNA
atggcccaggtgcagctggtgcagtctggagctgaggtgaagaagcctggggcctcagttaaagtttcctgtaaggctagtggttacacctttaccagctatggtatcagttgggtgaggcaggcccctggacaagggcttgaatggatgggatggatcagcgcttacaatggtaacacaaactatgcacagaagctccagggcagagtcaccatgaccacagacacatccactagcacagcatacatggagctgaggagtttgagatctgacgacacggccgtttattactgtgcgcggcgagattactatgatggtagtggatttgactactggggccaaggaactctggtcacagtctcgagcggtggaggcggttccggcggagggggcagcggcggtggcggatcaactcagtctgtgttgacgcagccgccctcagtgagtgcagccccaggccaaaaagtcaccatctcctgctctggaagcagctccaacattgggaataattacgtatcctggtaccagcagctcccaggcacagctcccaaattacttatttatgataataacaagcgtccctcagggattcctgaccggttctctggctccaaatctggcacgtcagccaccctgggcataactgggctccaaactggggacgaagccgattattactgcggaacttatgatggcgaagggcgccacgaggtgttcggcggagggaccaagctgaccgtactaggggcggca

SEQ ID NO: 8 - DNA, 762 bp, synthetic construct
  source 1..762: mol_type = other DNA
atggcccaggtgcaactggtggaatcgggcggaggcgtggtccagcctgggaggtccctgagactttcctgtgcagcctctggatttaccttcagtagctatgctatgcactgggtgcgccaagctccaggcaaggggctggaatgggttgcagttatatcatatgatggaagtaataaatactacgcagactccgtgaagggccgttttaccatctcccgggacaattccaaaaacactctgtatctgcaaatgaacagccttagagctgaagacactgccgtgtactactgcgcgcgcgatccgtggggtggttggttagggagcgatgcttttgacatttggggccaaggaaccttggtcacagtctcgagcggtggaggcggttcaggcggaggtggcagcggcggtggcggcagcacgcagtctgtgctgactcagccaccctcagcgtctgggacacccgggcagagggtcaccatttcttgttctggaagcagcagcaacattggaagtaacactgtaaactggtaccagcagttgccaggaacggcccccaaactcctcatctatagtaataatcagcggccttcaggggtacctgaccgattctccggctccaagtctggcacctcagcctccctcgccatcagtgggctccagtctgaggatgaggctgattattactgcgcagcatacgatggggacgggggggaggacgtgttcggcggaggtacaaagctgacagttctaggggccgcc

SEQ ID NO: 9 - DNA, 759 bp, synthetic construct
  source 1..759: mol_type = other DNA
atggccgaggtgcagctgttggaatctgggggaggcttggtacagccgggggggtccctgcgtctctcctgtgcagcctctggattcacctttagcagctatgctatgagctgggtgcgccaggctccagggaaggggctggaatgggtgtcagctattagtggtagtggtggcagcacatactacgcagattccgtgaaaggccggttcacaatctccagagataatagtaagaacacactgtaccttcaaatgaacagcttacgcgccgaggacacggcggtgtattactgtgcaagacgatatagcagtggctggtcatactactttgactactggggccagggaaccctggtcacagtctcgagcggtggaggcggttcaggcggaggtggcagcggcggtggcggatctacgcagtctgccctgactcaacctgcctccgtgtctgggtctcctggacagtcgatcaccatatcctgcactggaacatccagtgacgttggaggttataactatgtttcctggtaccaacagcacccaggcaaagcccccaaactcatgatttatgaagtaagtaatcggccctcaggggtttctaataggttctcaggctccaagagtggcaacactgcctccctgaccatctctgggctccaggctgaggacgaggctgattactactgcagctcatatgatacttttgagaggattagcgtgttcggcggagggaccaagcttaccgtcctaggggcggca

SEQ ID NO: 10 - DNA, 747 bp, synthetic construct
  source 1..747: mol_type = other DNA
atggcacaagtgcaactggtgcagtctggagctgaagtgaagaaaccgggggcctcagttaaggtctcctgcaaagcttctggttacacctttactagctatggtatcagctgggtgagacaagcccctggacaagggcttgagtggatgggctggatcagcgcttacaatggtaacacaaactatgcacagaagctccagggccgtgtcaccatgaccacagacacatccacctccacagcctacatggagctgaggagcctgagatcggacgacacggctgtgtattactgtgcgagacggagttatgatagtagtggacttgactactggggccagggaacattggttacagtttcgagcggcggcggcggttcaggcggagggggcagcggcggtggcggttctacgcagtccgtgttgactcagccaccctcagcgtctgggacccccgggcagagggtcaccatttcttgttctggaagcagctccaacattggaagtaatactgtaaactggtaccagcagctcccaggaactgcccctaaattactcatatatagtaataatcagcggccctcaggggtacctgaccgattctccggctccaagtctggcacctcagcctccctggccatcagtgggctgcagagcgaagatgaggctgattattactgcgcagcatgggatcgcaggtggcgcctggtgttcggcggagggaccaagctgactgtcctaggggccgcc

SEQ ID NO: 11 - DNA, 750 bp, synthetic construct
  source 1..750: mol_type = other DNA
atggcccaggtgcagcttgtgcagtccggtgctgaggtgaagaaacctggggcctcagttaaggtttcctgcaaggcttctggttacacctttaccagctatggtatcagctgggtgcgacaggcccctggacaagggcttgagtggatgggctggataagcgcttacaatggtaacacaaactatgcacaaaagttgcagggccgtgtcaccatgaccacagacacatccacaagcacagcctacatggagctgaggtccttgagatctgacgacacggccgtgtattactgcgcaagacaggtgggcgctccgactcgctttgactactggggccagggaaccctggtgacagtctcgagcggtggaggcgggtcaggcggaggcggcagcggcggtggcggatcgacgcagtctgtgctgactcaaccaccatcagcgtccgggacccccgggcaaagggtcactattagttgtagtggaagcagctccaacattggaagtaatactgtaaactggtaccagcagctcccaggaactgcccccaaactcttaatctatagtaataatcagcggccctcaggggttcctgatcggttctctggctccaagtctggcacctcagcctccctggcaatcagtgggctccagtctgaagatgaagctgattattactgtgcagcatgggatacgcacgcctgggcccccgtattcggcggagggactaaactgaccgtcctaggggcggct

SEQ ID NO: 12 - DNA, 753 bp, synthetic construct
  source 1..753: mol_type = other DNA
atggcccaggtgcaactggtgcagtctggggctgaggtcaagaagccagggtcctcggtgaaggtctcctgtaaggctagtggaggcaccttcagcagctatgctatcagctgggtgagacaagcccctggacaagggcttgagtggatgggagggatcatccctatatttggtacagcaaactacgcacagaaatttcagggcagagttacgataactgcagacgaatccactagcacagcatacatggagctgagtagtttaaggtctgaagacactgcagtgtattactgtgctcgttctaaatataactgggcctacaaaaatgattactggggccagggaaccctggttacagtttcaagcggtggaggcggttcaggcggaggtggcagcggcggtggcggctctacacagagcgtgttgacgcagccgccctcagtatcggcggccccagggcagaaggtcaccatctcctgctctggaagcagttccaacattgggaataactatgtatcctggtaccagcagctcccaggtacagcccccaaattgctcatttacgacaataataagcgaccctcagggattcctgatcgcttcagtggctccaaatctggcacctcagccaccctgggcatcaccggacttcaaactggggacgaagctgattattactgcggaacatatgatccctgggctcggactgccgtgttcggcggagggaccaagctgaccgtcctaggggcggcc

SEQ ID NO: 13 - DNA, 732 bp, synthetic construct
  source 1..732: mol_type = other DNA
caggttaaactccaacaaagtggcggaggcttggttcagcctggagggagtatgaaactgtcttgtgtcgtatctggttttacattctcaaattattggatgaattgggttaggcaatcaccggagaagggattggaatggatcgctgagattcggttgaaatcaaacaacttcggtcgctattatgcggaatccgtgaaaggtcggttcacgatttcccgcgacgattcaaagtccagtgcttatctgcaaatgattaatcttcgggcagaagatacaggaatatactattgtacctcctatggtaactatgttggtcactatttcgatcattgggggcagggaaccactgtcaccgtatccagcggtggcggagggagcggcggtggaggaagcggaggcggaggttccgacattgaactgactcaatctcccaaatttatgtcaacgagcgtcggggaccgcgtgagcgttacgtgtaaggcttcacaaaacgtagacaccaatgtggcctggtatcaacaaaagccgggacaatctccagagcccctgctcttttcagcaagttacaggtacaccggtgttccagatagattcacaggtagtggatctggtactgattttactctcaccataagtaacgtgcagtccgaagacctcgccgagtacttttgtcaacagtataatagttacccacttacatttgggggtggaacaaaactggaaatcaag

SEQ ID NO: 14 - DNA, 798 bp, synthetic construct
  source 1..798: mol_type = other DNA
atggcccaggtacaactgcaacagtcagggccaggactggttaagccctcgcagaccttatcacttacctgtgccatctccggggacagtgtctctagcaacagtgctgcttggaactggataaggcagtccccaagccgcggccttgaatggctgggaagaacatactacaggtccaagtggtataatgattatgcagttagtgtgaaaagtcgaataactatcaaccctgatacatccaagaaccagttctccctgcagctgaactctgtgactcctgaggacacagctgtgtattactgtgcgagagatcagcggaattacgatttttggagtggttattatccgcccgcagaattagggtactacggtatggacgtctggggccagggaaccctggtcacagtctcgagcggtggaggcggttcaggcggaggcggcagcggcggtggcggctctacgcagtctgtgttgacgcagccgcctagcgtgtctgctgccccaggtcagaaagtgaccatctcctgctctggaagcagctccaacattgggaataattatgtatcctggtaccagcaactcccaggaacagcacccaaactcctcatttatgacaataataagcggccctcagggattcctgaccgtttttctggcagtaaaagcggcacttcagccactctgggcatcaccgggctccaaactggggacgaggccgattactactgcggaacatgggattggagcgcattggtggtgttcggcggagggaccaagctgaccgttctaggggcggcc

SEQ ID NO: 15 - DNA, 753 bp, synthetic construct
  source 1..753: mol_type = other DNA
atggcccaggtgcagctggtggagtctgggggtggcgtggtccagcctgggaggtccttgcgtctctcctgcgcagcctctggattcactttcagtagctatgctatgcactgggtccgccaagctccgggcaaggggctggaatgggtggcagttataagctatgatggaagtaataaatactacgcagactccgtgaagggccgattcaccatctccagagacaattccaagaacacgctttatctgcaaatgaacagccttcgcgctgaagacacagctgtgtattactgtgccagagtatcagactggaacgacgccgcttttgatatttggggccagggaactctggttacagtctcgagcggtggaggcggttcaggcggaggtggcagcggcggtggcggatcgacccagtctgtgctgactcagccaccctcagcgagcgggacaccagggcagcgggtcaccatttcttgttctggaagcagctccaacatcggtagtaatactgtaaactggtaccagcaactgccaggaacggcccccaaactcctcatctacagtaataatcaacggccttcaggggttcctgatagattttctggctccaaaagcggcacctcagcctccctggccattagtgggttacagtctgaagatgaggctgattattactgcgcagcatgggaccccgagaccgccaggtgggtgtttggcggagggacaaagttgaccgtcctaggggcggcc

SEQ ID NO: 16 - DNA, 774 bp, synthetic construct
  source 1..774: mol_type = other DNA
atggcccaggtacagttgcagcaatcaggtccaggactggtgaagcccagccaaaccttatcattaacttgtgcaatctccggggacagtgtttctagcaacagtgctgcttggaactggatcaggcagtccccttcgagaggccttgagtggctgggaaggacatactaccggagcaagtggtataatgattatgcagttagcgtgaaaagtcggataaccatcaaccctgacacatccaagaaccagttctccctgcaactgaactctgtgactcccgaagacacggctgtgtattactgtgcacgcagaggggaacactatgatagtagtggttattactacggccttgattactggggccagggaaccctggtcacagtcagcagcggtggaggcggttcaggcggcggtggcagcggcggtggcggatctacgcagtcggtgttgacacagccgccctcagtgtctgcggccccagggcaaaaagttaccatatcctgctctggaagcagctccaacattgggaataattatgtatcctggtaccagcagctcccaggaacagcccccaaactcctcatttatgacaataataagcgtccttcagggattcctgaccgattctctggctccaagtctggcacttcagccactctgggcatcaccggactccagactggggacgaggccgattactactgcggaacatatgatgtcgcggctgggtacgtgtttggcggagggaccaaactgaccgtcctaggggcagcc

SEQ ID NO: 17 - DNA, 762 bp, synthetic construct
  source 1..762: mol_type = other DNA
atggcccaggtacagctggtgcagagcggggctgaggtgaaaaagcccgggtcctcggtgaaagtgtcctgcaaggcttctggaggcaccttcagcagctatgctataagctgggtgcgacaagccccgggacaagggcttgagtggatgggagggatcatccctatctttggtacagcaaactacgcacagaagttccagggcagagtcaccattaccgcggacgaatccacgagcacagcctacatggaactgagcagccttaggtctgaggacactgccgtgtattactgtgcgagaccacgtactgcaggctggagttatgatgcttttgatgcctggggccagggaacattggtaacagtctcaagcggtggaggcggttcaggcggaggtggcagcggcggtggcggatctacgcaatctgccttaactcagcctgccagtgtgtctgggagtcctggacagtcaataaccatttcctgtactggaaccagcagtgacgttggcggttataactacgtttcctggtaccaacagcacccaggcaaagcccccaaactcatgatttacgaagtcagtaataggccctcaggggtttctaatcgcttctctggctccaagtcaggcaacacagcctccctgactatctctgggctccaggctgaggacgaggctgattattactgcagttcatatgattggcggtcctccgggtcggtgtttggcggagggaccaagctgaccgtcctaggggcagca

SEQ ID NO: 18 - DNA, 726 bp, synthetic construct
  source 1..726: mol_type = other DNA
gaggtacagctagtggagtcaggcgggggcctggtccagccaggcggatcgttaagacttagttgcgcagcaagcgggtttacgttctcaggtagccgaatggggtgggtgagacaggcccccgggaaaggactcgaatgggtttccgcgatcaacgaagaaggtggacaaacttactacgccgatagcgtgaagggacggtttacaatttctcgtgacaactccaagaataccctgtatctgcaaatgaatagtttgagggctgaggacaccgccgtctattattgtgctaaacatcctccgcaccgccccttcgattactggggccagggcacactcgtgactgtttctaggggtggaggcggttccggcggagggggcagcggcggtggcggatcatcttccgaattaactcaagacccagccgtttctgtggccctcggccagacagtcaggatcacgtgccaaggggatagtctgcgatcctactatgcaagctggtaccagcagaaaccgggccaggctcctgtattggtgatctacggaaagaataacagaccctctggtatacccgaccggttctccggtagcagtagcggcaacaccgcttcacttactattaccggagcacaggccgaggatgaggcggactattattgtaattcatcgcacggcccacgtcgccctgtcgtgtttgggggaggcaccaagctgacagtgctgggg

SEQ ID NO: 19 - DNA, 720 bp, synthetic construct
  source 1..720: mol_type = other DNA
gaggtgcagctggtcgagagcggtggaggcctcgtgcagcccggagggtcattgcgtctctcctgcgccgccagcggctttaccttcggtcaatatagcatgagttgggtcaggcaggcgcctggcaagggactggaatgggtttccgccatcaccggtacaggaggggaaacatactacgctgactcagtagaggggagattcactatttctcgagataactccaagaacacgctgtatctacaaatgaattctttacgcgcagaagacactgcagtttattactgtgctaaagggagacggatatttgattactggggccagggcacccttgtgacagtgagtaggggtggaggcggttccggcggagggggcagcggcggtggcggatcatcctccgaactgacacaggacccggctgtgagcgtcgcccttggtcagactgtgagaattacatgccagggggattcactcaggcggcagcctgcttcttggtaccaacaaaagcctggacaggcccccgtgttggtgatatactacaaaaagctgcgaccatcaggaatccctgaccgcttttctggaagctccagtgggaataccgcaagtctcaccatcactggtgcccaggcagaggatgaggcggactattattgtaactcgttcagccccaaacccaaaccagtagtcttcggcggcgggaccaagctgacggttttaggc

SEQ ID NO: 20 - DNA, 720 bp, synthetic construct
  source 1..720: mol_type = other DNA
gaggtccaattacttgaatcaggcggaggcctggtgcagcctggaggcagcctgagactgtcctgcgcggcaagcggtttcactttctccagatatggcgcatcctgggttaggcaggcacccggtaaaggactggagtgggtatctgccatttctgggagtggagggagtacctactacgctgattcggtgaaggggcgttttacaatctcacgagacaatagcaaaaacacactatatctccagatgaattctctccgcgccgaagacacggctgtctactattgtgctaaggcccacaacgcctttgattactggggccaggggaccttggtgactgtgagccggggtggaggcggttccggcggagggggcagcggcggtggcggatcatcgagcgaactgacacaggacccggcggtttccgtggcactggggcagacagtaagaataacttgtcaaggggatagcctgcgcagttactacgccagctggtaccagcagaaaccaggccaggcccccgttttggtgatttatgggaagaataacaggccttccggcatccccgaccggttttctggatctagttctggaaacaccgcatcacttaccatcacgggagctcaagccgaggatgaggctgactactattgcaattcatccgtctatactatgcctccagtggtgttcggtggcggtacaaagttaaccgtcctcggc

SEQ ID NO: 21 - protein, 255 aa, synthetic construct
MAQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGSGYSYGPGYDAFDIWGQGTLVTVSSGGGGSGGGGSGGGGSTNFMLTQPHSVSESPGKTVTISCTRSSGSIASNYVQWYQQRPGSSPTTVIYEDNQRPSGVPDRFSGSIDSSSNSASLTISGLKTEDEADYYCQSWDPVFGVFGGGTKLTVLGAA

SEQ ID NO: 22 - protein, 254 aa, synthetic construct
MAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREGGAVGYYYGMDVWGQGTLVTVSSGGGGSGGGGSGGGGSTQSALTQPASVSGSPGQSITISCTGTSSDVGGYNYVSWYQQHPGKAPKLMIYEVSNRPSGVSNRFSGSKSGNTASLTISGLQAEDEADYYCSSYDRSNRSMVFGGGTKLTVLGAA

SEQ ID NO: 23 - protein, 253 aa, synthetic construct
MAEVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARPGYGDFPGAFDIWGQGTLVTVSSGGGGSGGGGSGGGGSTQSALTQPASVSGSPGQSITISCTGTSSDVGGYNYVSWYQQHPGKAPKLMIYEVSNRPSGVSNRFSGSKSGNTASLTISGLQAEDEADYYCSSYDWATYGSVFGGGTKLTVLGAA

SEQ ID NO: 24 - protein, 251 aa, synthetic construct
MAQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDQDDSSDAFDIWGQGTLVTVSSGGGGSGGGGSGGGGSTQSVLTQPPSVSAAPGQKVTISCSGSSSNIGNNYVSWYQQLPGTAPKLLIYDNNKRPSGIPDRFSGSKSGTSATLGITGLQTGDEADYYCGTYDYIATRAVFGGGTKLTVLGAA

SEQ ID NO: 25 - protein, 256 aa, synthetic construct
MAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGTYYDFWSGYYDAFDIWGQGTLVTVSSGGGGSGGGGSGGGGSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSYWQPVFGGGTKLTVLGAA

SEQ ID NO: 26 - protein, 251 aa, synthetic construct
MAQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDQDDSSDAFDIWGQGTLVTVSSGGGGSGGGGSGGGGSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDYKVSRLVFGGGTKLTVLGAA

SEQ ID NO: 27 - protein, 251 aa, synthetic construct
MAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARRDYYDGSGFDYWGQGTLVTVSSGGGGSGGGGSGGGGSTQSVLTQPPSVSAAPGQKVTISCSGSSSNIGNNYVSWYQQLPGTAPKLLIYDNNKRPSGIPDRFSGSKSGTSATLGITGLQTGDEADYYCGTYDGEGRHEVFGGGTKLTVLGAA

SEQ ID NO: 28 - protein, 254 aa, synthetic construct
MAQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDPWGGWLGSDAFDIWGQGTLVTVSSGGGGSGGGGSGGGGSTQSVLTQPPSASGTPGQRVTISCSGSSSNIGSNTVNWYQQLPGTAPKLLIYSNNQRPSGVPDRFSGSKSGTSASLAISGLQSEDEADYYCAAYDGDGGEDVFGGGTKLTVLGAA

SEQ ID NO: 29 - protein, 253 aa, synthetic construct
MAEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARRYSSGWSYYFDYWGQGTLVTVSSGGGGSGGGGSGGGGSTQSALTQPASVSGSPGQSITISCTGTSSDVGGYNYVSWYQQHPGKAPKLMIYEVSNRPSGVSNRFSGSKSGNTASLTISGLQAEDEADYYCSSYDTFERISVFGGGTKLTVLGAA

SEQ ID NO: 30 - protein, 249 aa, synthetic construct
MAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARRSYDSSGLDYWGQGTLVTVSSGGGGSGGGGSGGGGSTQSVLTQPPSASGTPGQRVTISCSGSSSNIGSNTVNWYQQLPGTAPKLLIYSNNQRPSGVPDRFSGSKSGTSASLAISGLQSEDEADYYCAAWDRRWRLVFGGGTKLTVLGAA

SEQ ID NO: 31 - protein, 250 aa, synthetic construct
MAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARQVGAPTRFDYWGQGTLVTVSSGGGGSGGGGSGGGGSTQSVLTQPPSASGTPGQRVTISCSGSSSNIGSNTVNWYQQLPGTAPKLLIYSNNQRPSGVPDRFSGSKSGTSASLAISGLQSEDEADYYCAAWDTHAWAPVFGGGTKLTVLGAA

SEQ ID NO: 32 - protein, 251 aa, synthetic construct
MAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSKYNWAYKNDYWGQGTLVTVSSGGGGSGGGGSGGGGSTQSVLTQPPSVSAAPGQKVTISCSGSSSNIGNNYVSWYQQLPGTAPKLLIYDNNKRPSGIPDRFSGSKSGTSATLGITGLQTGDEADYYCGTYDPWARTAVFGGGTKLTVLGAA

SEQ ID NO: 33 - protein, 244 aa, synthetic construct
QVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSSGGGGSGGGGSGGGGSDIELTQSPKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIK

SEQ ID NO: 34 - protein, 266 aa, synthetic construct
MAQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARDQRNYDFWSGYYPPAELGYYGMDVWGQGTLVTVSSGGGGSGGGGSGGGGSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDWSALVVFGGGTKLTVLGAA

SEQ ID NO: 35 - protein, 251 aa, synthetic construct
MAQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVSDWNDAAFDIWGQGTLVTVSSGGGGSGGGGSGGGGSTQSVLTQPPSASGTPGQRVTISCSGSSSNIGSNTVNWYQQLPGTAPKLLIYSNNQRPSGVPDRFSGSKSGTSASLAISGLQSEDEADYYCAAWDPETARWVFGGGTKLTVLGAA

SEQ ID NO: 36 - protein, 258 aa, synthetic construct
MAQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARRGEHYDSSGYYYGLDYWGQGTLVTVSSGGGGSGGGGSGGGGSTQSVLTQPPSVSAAPGQKVTISCSGSSSNIGNNYVSWYQQLPGTAPKLLIYDNNKRPSGIPDRFSGSKSGTSATLGITGLQTGDEADYYCGTYDVAAGYVFGGGTKLTVLGAA

SEQ ID NO: 37 - protein, 254 aa, synthetic construct
MAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARPRTAGWSYDAFDAWGQGTLVTVSSGGGGSGGGGSGGGGSTQSALTQPASVSGSPGQSITISCTGTSSDVGGYNYVSWYQQHPGKAPKLMIYEVSNRPSGVSNRFSGSKSGNTASLTISGLQAEDEADYYCSSYDWRSSGSVFGGGTKLTVLGAA

SEQ ID NO: 38 - protein, 242 aa, synthetic construct
EVQLVESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSRGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSSHGPRRPVVFGGGTKLTVLG

SEQ ID NO: 39 - protein, 240 aa, synthetic construct
EVQLVESGGGLVQPGGSLRLSCAASGFTFGQYSMSWVRQAPGKGLEWVSAITGTGGETYYADSVEGRFTISRDNSKNTLYLQMNSLRAEDTAVYYCAKGRRIFDYWGQGTLVTVSRGGGGSGGGGSGGGGSSSELTQDPAVSVALGQTVRITCQGDSLRRQPASWYQQKPGQAPVLVIYYKKLRPSGIPDRFSGSSSGNTASLTITGAQAEDEADYYCNSFSPKPKPVVFGGGTKLTVLG

SEQ ID NO: 40 - protein, 240 aa, synthetic construct
EVQLLESGGGLVQPGGSLRLSCAASGFTFSRYGASWVRQAPGKGLEWVSAISGSGGSTYYADSVKGRFTISRDNSKNTLYLQMNSLRAEDTAVYYCAKAHNAFDYWGQGTLVTVSRGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVLG

SEQ ID NO: 41 - protein, 126 aa, synthetic construct
MAQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGSGYSYGPGYDAFDIWGQGTLVTVSS

SEQ ID NO: 42 - protein, 114 aa, synthetic construct
TNFMLTQPHSVSESPGKTVTISCTRSSGSIASNYVQWYQQRPGSSPTTVIYEDNQRPSGVPDRFSGSIDSSSNSASLTISGLKTEDEADYYCQSWDPVFGVFGGGTKLTVLGAA

SEQ ID NO: 43 - protein, 124 aa, synthetic construct
MAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREGGAVGYYYGMDVWGQGTLVTVSS

SEQ ID NO: 44 - protein, 115 aa, synthetic construct
TQSALTQPASVSGSPGQSITISCTGTSSDVGGYNYVSWYQQHPGKAPKLMIYEVSNRPSGVSNRFSGSKSGNTASLTISGLQAEDEADYYCSSYDRSNRSMVFGGGTKLTVLGAA

SEQ ID NO: 45 - protein, 123 aa, synthetic construct
MAEVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARPGYGDFPGAFDIWGQGTLVTVSS

SEQ ID NO: 46 - protein, 115 aa, synthetic construct
TQSALTQPASVSGSPGQSITISCTGTSSDVGGYNYVSWYQQHPGKAPKLMIYEVSNRPSGVSNRFSGSKSGNTASLTISGLQAEDEADYYCSSYDWATYGSVFGGGTKLTVLGAA

SEQ ID NO: 47 - protein, 122 aa, synthetic construct
MAQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDQDDSSDAFDIWGQGTLVTVSS

SEQ ID NO: 48 - protein, 114 aa, synthetic construct
TQSVLTQPPSVSAAPGQKVTISCSGSSSNIGNNYVSWYQQLPGTAPKLLIYDNNKRPSGIPDRFSGSKSGTSATLGITGLQTGDEADYYCGTYDYIATRAVFGGGTKLTVLGAA

SEQ ID NO: 49 - protein, 127 aa, synthetic construct
MAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGTYYDFWSGYYDAFDIWGQGTLVTVSS

SEQ ID NO: 50 - protein, 114 aa, synthetic construct
TQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSYWQPVFGGGTKLTVLGAA

SEQ ID NO: 51 - protein, 122 aa, synthetic construct; identical to SEQ ID NO: 47

SEQ ID NO: 52 - protein, 114 aa, synthetic construct
TQSVLTQPPSVSAAPGQKVTISCSGSSSNIGNNYVSWYQQLPGTAPKLLIYDNNKRPSGIPDRFSGSKSGTSATLGITGLQTGDEADYYCGTWDYKVSRLVFGGGTKLTVLGAA

SEQ ID NO: 53 - protein, 122 aa, synthetic construct
MAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARRDYYDGSGFDYWGQGTLVTVSS

SEQ ID NO: 54 - protein, 114 aa, synthetic construct
TQSVLTQPPSVSAAPGQKVTISCSGSSSNIGNNYVSWYQQLPGTAPKLLIYDNNKRPSGIPDRFSGSKSGTSATLGITGLQTGDEADYYCGTYDGEGRHEVFGGGTKLTVLGAA

SEQ ID NO: 55 - protein, 125 aa, synthetic construct
MAQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDPWGGWLGSDAFDIWGQGTLVTVSS

SEQ ID NO: 56 - protein, 114 aa, synthetic construct
TQSVLTQPPSASGTPGQRVTISCSGSSSNIGSNTVNWYQQLPGTAPKLLIYSNNQRPSGVPDRFSGSKSGTSASLAISGLQSEDEADYYCAAYDGDGGEDVFGGGTKLTVLGAA

SEQ ID NO: 57 - protein, 123 aa, synthetic construct
MAEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARRYSSGWSYYFDYWGQGTLVTVSS

SEQ ID NO: 58 - protein, 115 aa, synthetic construct
TQSALTQPASVSGSPGQSITISCTGTSSDVGGYNYVSWYQQHPGKAPKLMIYEVSNRPSGVSNRFSGSKSGNTASLTISGLQAEDEADYYCSSYDTFERISVFGGGTKLTVLGAA

SEQ ID NO: 59 - protein, 121 aa, synthetic construct
MAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARRSYDSSGLDYWGQGTLVTVSS

SEQ ID NO: 60 - protein, 113 aa, synthetic construct
TQSVLTQPPSASGTPGQRVTISCSGSSSNIGSNTVNWYQQLPGTAPKLLIYSNNQRPSGVPDRFSGSKSGTSASLAISGLQSEDEADYYCAAWDRRWRLVFGGGTKLTVLGAA

SEQ ID NO: 61 - protein, 121 aa, synthetic construct
MAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARQVGAPTRFDYWGQGTLVTVSS

SEQ ID NO: 62 - protein, 114 aa, synthetic construct
TQSVLTQPPSASGTPGQRVTISCSGSSSNIGSNTVNWYQQLPGTAPKLLIYSNNQRPSGVPDRFSGSKSGTSASLAISGLQSEDEADYYCAAWDTHAWAPVFGGGTKLTVLGAA

SEQ ID NO: 63 - protein, 122 aa, synthetic construct
MAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSKYNWAYKNDYWGQGTLVTVSS

SEQ ID NO: 64 - protein, 114 aa, synthetic construct
TQSVLTQPPSVSAAPGQKVTISCSGSSSNIGNNYVSWYQQLPGTAPKLLIYDNNKRPSGIPDRFSGSKSGTSATLGITGLQTGDEADYYCGTYDPWARTAVFGGGTKLTVLGAA

SEQ ID NO: 65 - protein, 122 aa, synthetic construct
QVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSS

SEQ ID NO: 66 - protein, 107 aa, synthetic construct
DIELTQSPKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIK

SEQ ID NO: 67 - protein, 138 aa, synthetic construct
MAQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARDQRNYDFWSGYYPPAELGYYGMDVWGQGTLVTVSS

SEQ ID NO: 68 - protein, 113 aa, synthetic construct
TQSVLTQPPSVSAAPGQKVTISCSGSSSNIGNNYVSWYQQLPGTAPKLLIYDNNKRPSGIPDRFSGSKSGTSATLGITGLQTGDEADYYCGTWDWSALVVFGGGTKLTVLGAA

SEQ ID NO: 69 - protein, 122 aa, synthetic construct
MAQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVSDWNDAAFDIWGQGTLVTVSS

SEQ ID NO: 70 - protein, 114 aa, synthetic construct
TQSVLTQPPSASGTPGQRVTISCSGSSSNIGSNTVNWYQQLPGTAPKLLIYSNNQRPSGVPDRFSGSKSGTSASLAISGLQSEDEADYYCAAWDPETARWVFGGGTKLTVLGAA

SEQ ID NO: 71 - protein, 130 aa, synthetic construct
MAQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARRGEHYDSSGYYYGLDYWGQGTLVTVSS

SEQ ID NO: 72 - protein, 113 aa, synthetic construct
TQSVLTQPPSVSAAPGQKVTISCSGSSSNIGNNYVSWYQQLPGTAPKLLIYDNNKRPSGIPDRFSGSKSGTSATLGITGLQTGDEADYYCGTYDVAAGYVFGGGTKLTVLGAA

SEQ ID NO: 73 - protein, 124 aa, synthetic construct
MAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARPRTAGWSYDAFDAWGQGTLVTVSS

SEQ ID NO: 74 - protein, 115 aa, synthetic construct
TQSALTQPASVSGSPGQSITISCTGTSSDVGGYNYVSWYQQHPGKAPKLMIYEVSNRPSGVSNRFSGSKSGNTASLTISGLQAEDEADYYCSSYDWRSSGSVFGGGTKLTVLGAA

SEQ ID NO: 75 - protein, 118 aa, synthetic construct
EVQLVESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSR

SEQ ID NO: 76 - protein, 109 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSSHGPRRPVVFGGGTKLTVLG

SEQ ID NO: 77 - protein, 116 aa, synthetic construct
EVQLVESGGGLVQPGGSLRLSCAASGFTFGQYSMSWVRQAPGKGLEWVSAITGTGGETYYADSVEGRFTISRDNSKNTLYLQMNSLRAEDTAVYYCAKGRRIFDYWGQGTLVTVSR

SEQ ID NO: 78 - protein, 109 aa, synthetic construct
SSELTQDPAVSVALGQTVRITCQGDSLRRQPASWYQQKPGQAPVLVIYYKKLRPSGIPDRFSGSSSGNTASLTITGAQAEDEADYYCNSFSPKPKPVVFGGGTKLTVLG

SEQ ID NO: 79 - protein, 116 aa, synthetic construct
EVQLLESGGGLVQPGGSLRLSCAASGFTFSRYGASWVRQAPGKGLEWVSAISGSGGSTYYADSVKGRFTISRDNSKNTLYLQMNSLRAEDTAVYYCAKAHNAFDYWGQGTLVTVSR

SEQ ID NO: 80 - protein, 109 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVLG

SEQ ID NO: 81 - protein, 15 aa, synthetic construct
GGGGSGGGGSGGGGS

SEQ ID NO: 82 - protein, 19 aa, synthetic construct
SSSGGGGSGGGGSGGGGSS

SEQ ID NO: 83 - protein, 118 aa, synthetic construct
EVQLQESGGGFVQAGGSLRLSCAASGRLRPFERMGWFRQAPGKEREFVAAISVHDEVFPYYADSVKGRFTISRDNAKNTVYLQMNSLKPEDTATYYCAFFIMDDVKYWGQGTQVTVSS

SEQ ID NO: 84 - protein, 118 aa, synthetic construct
EVQLQESGGGFVQAGGSLRLSCAASGESFVPEMGWFRQAPGKEREFVAAISVEQSLDMYYADSVKGRFTISRDNAKNTVYLQMNSLKPEDTATYYCAIVAIDDFNTYWGQGTQVTVSS

SEQ ID NO: 85 - protein, 119 aa, synthetic construct
EVQLQESGGGFVQAGGSLRLSCAASGVSLRTRSMGWFRQAPGKEREFVAAISRHNDHEFYYADSVKGRFTISRDNAKNTVYLQMNSLKPEDTATYYCAKLPVNPNLHYWGQGTQVTVSS

SEQ ID NO: 86 - protein, 119 aa, synthetic construct
EVQLQESGGGFVQAGGSLRLSCAASGVHVPLQNMGWFRQAPGKEREFVAAISRDMPVDNYYADSVKGRFTISRDNAKNTVYLQMNSLKPEDTATYYCAVRVYTTSLWYWGQGTQVTVSS

SEQ ID NO: 87 - protein, 119 aa, synthetic construct
EVQLQESGGGFVQAGGSLRLSCAASGNVQRRFRMGWFRQAPGKEREFVAAISTNRDRRNYYADSVKGRFTISRDNAKNTVYLQMNSLKPEDTATYYCAVMNKNFTYMYWGQGTQVTVSS

SEQ ID NO: 88 - protein, 119 aa, synthetic construct
EVQLQESGGGFVQAGGSLRLSCAASGEPVHSTSMGWFRQAPGKEREFVAAISLNVMHSRYYADSVKGRFTISRDNAKNTVYLQMNSLKPEDTATYYCASYPHYMTPMYWGQGTQVTVSS

SEQ ID NO: 89 - protein, 122 aa, synthetic construct
EVQLQESGGGFVQAGGSLRLSCAASGSDVTRLNMGWFRQAPGKEREFVAAISRSEQNRLYYADSVKGRFTISRDNAKNTVYLQMNSLKPEDTATYYCATTALAAVTKATHYWGQGTQVTVSS

SEQ ID NO: 90 - protein, 125 aa, synthetic construct
EVQLQESGGGFVQAGGSLRLSCAASGNVVFLTSMGWFRQAPGKEREFVAAISRSFFDDPYYADSVKGRFTISRDNAKNTVYLQMNSLKPEDTATYYCAPYKRDYRQHTVPRHIYWGQGTQVTVSS

SEQ ID NO: 91 - protein, 122 aa, synthetic construct
EVQLQESGGGFVQAGGSLRLSCAASGSSSRLFNMGWFRQAPGKEREFVAAISHMENDVDYYADSVKGRFTISRDNAKNTVYLQMNSLKPEDTATYYCALMLKGWDHSTRYYWGQGTQVTVSS

SEQ ID NO: 92 - protein, 119 aa, synthetic construct
EVQLQESGGGFVQAGGSLRLSCAASGNLMVRREMGWFRQAPGKEREFVAAISRSSQEEVYYADSVKGRFTISRDNAKNTVYLQMNSLKPEDTATYYCAMEGFYVYNQYWGQGTQVTVSS

SEQ ID NO: 93 - protein, 119 aa, synthetic construct
EVQLQESGGGFVQAGGSLRLSCAASGRRVTVSEMGWFRQAPGKEREFVAAISMRERESMYYADSVKGRFTISRDNAKNTVYLQMNSLKPEDTATYYCAEYTHWYSHPYWGQGTQVTVSS

SEQ ID NO: 94 - DNA, 354 bp, synthetic construct
  source 1..354: mol_type = other DNA
gaggttcaactgcaggagagcggcggtggatttgtgcaagcaggggggtccttgcgattatcatgtgctgcatcggggcggctgagaccattcgaacgtatgggctggttccgccaggcacctggtaaagagagggagtttgtggccgccataagcgtgcacgatgaagtgtttccgtactacgccgattccgtcaagggcaggtttaccattagtagagacaatgcgaaaaacacagtctatcttcagatgaactctctcaagcccgaagatactgctacatattactgcgctttcttcatcatggacgacgttaagtattggggacagggaacccaggtaacggtgtcatct

SEQ ID NO: 95 - DNA, 354 bp, synthetic construct
  source 1..354: mol_type = other DNA
gaggtgcaattgcaggagagcggcggtggatttgtgcaggccggagggtccctccgactttcatgcgctgcatctggggaaagcttcgttccagaaatgggctggttccgccaggcacctggtaaagagcgggagtttgtggccgccataagcgtagaacagagtctggatatgtattacgccgattccgtcaagggcaggtttaccattagtagagacaatgcgaaaaacactgtctatctgcagatgaattctttaaagcccgaggatactgctacatactactgtgctatcgttgctatcgacgacttcaacacatattggggacaggggacccaagtgacggtgtcatcc

SEQ ID NO: 96 - DNA, 357 bp, synthetic construct
  source 1..357: mol_type = other DNA
gaggtgcaattgcaggagagtggcgggggatttgtgcaagcaggagggtccctccgactttcatgcgctgcatctggtgtgagcctgagaacccggagcatgggctggttccgccaggcaccaggtaaagagagggaatttgttgccgccatcagccgtcacaacgatcatgaattctattacgccgattccgtcaagggcaggttcaccattagtagagacaatgctaagaacacagtctatttacagatgaattccctcaagcccgaagacactgctacgtactactgtgctaaactgcctgttaacccgaatctgcactattggggacaggggacacaggtaactgtgtcatct

SEQ ID NO: 97 - DNA, 357 bp, synthetic construct
  source 1..357: mol_type = other DNA
gaggtgcaattacaggaaagcggcggtggatttgtccaggctggggggtccctccgactgtcatgtgctgcaagcggtgtgcacgtccctctgcagaacatgggctggttccgccaagcaccggggaaagagagggagttcgttgccgccattagccgggatatgccagtcgataactattacgccgactccgtgaagggcaggtttaccatcagtagagacaatgcgaaaaacacggtgtaccttcagatgaatagtttgaagcccgaagacactgctacatattactgcgctgttcgtgtgtacactacctctctgtggtattggggacagggaacccaggtaacagtgtcatct

SEQ ID NO: 98 - DNA, 357 bp, synthetic construct
  source 1..357: mol_type = other DNA
gaggtgcagctgcaggagagcggcgggggatttgtccaagctggtgggtccttgcgactgtcatgcgctgcatcgggtaacgttcagagacggttcagaatgggctggttccgccaggcacctgggaaagagagggaatttgttgccgccattagcacaaaccgggataggcgcaactattacgccgactccgtcaagggcaggtttaccatcagtcgtgacaatgcgaaaaacacggtgtaccttcagatgaattctctcaagcccgaagatactgctacatattactgtgctgtgatgaataagaatttcacttacatgtattggggacagggaacccaagtaaccgtgtcatct

SEQ ID NO: 99 - DNA, 357 bp, synthetic construct
  source 1..357: mol_type = other DNA
gaggtacagcttcaagaaagcggcgggggattcgtccaggcgggtgggtccttgcggctgtcatgcgctgcatctggggaaccagtgcacagtacaagtatgggctggttccgccaagcaccgggtaaagagagggagtttgttgccgccatcagcctgaacgttatgcacagcagatattacgccgattccgtcaagggccgatttaccatttcgcgtgacaatgcaaaaaacaccgtgtatttacagatgaattccctcaagcccgaagacactgctacgtattactgtgcttcttaccctcattacatgactcccatgtattggggacagggaacccaggtgacagtgtcatct

SEQ ID NO: 100 - DNA, 366 bp, synthetic construct
  source 1..366: mol_type = other DNA
gaggtgcaattacaggaaagcggcggtggatttgtccaggcaggtgggtccctccgactctcatgcgccgcatctgggagcgacgtcacacgtctgaacatgggctggttccgccaggcacctgggaaggagagggagtttgttgccgccattagcagaagtgaacagaaccggctgtactacgccgattccgtgaagggcaggttcaccatcagtagagacaatgcgaaaaacaccgtttatcttcagatgaattctttgaagcccgaagatactgctacctattactgtgcaacaacagctctggctgctgtgactaaagccactcactattggggacagggaacccaagtaacggtgtcatcc

SEQ ID NO: 101 - DNA, 375 bp, synthetic construct
  source 1..375: mol_type = other DNA
gaggtacagctgcaagagagcggcgggggatttgtccaggctggtgggtccctccgactttcatgtgctgcatctggtaacgttgtgttcctgacgtcgatgggctggttccgccaggcacccgggaaggagagggaatttgttgccgccataagccggagcttcttcgatgatccatattacgccgattccgtcaagggcaggtttaccattagtagagacaatgcaaaaaacacagtgtatttacagatgaatagtttgaagcccgaagacactgctacatattactgcgctccgtacaaaagagactaccggcagcacactgtgcctcgtcatatctattggggacagggaacccaagtgaccgtgtcatct

SEQ ID NO: 102 - DNA, 366 bp, synthetic construct
  source 1..366: mol_type = other DNA
gaggtgcagcttcaagagagcggcgggggatttgtccaagctggtgggtccctccgattatcatgcgctgcatctgggagcagtagtagactgttcaacatgggctggttccgccaggcacctggtaaagagcgggaatttgtggccgccattagccacatggaaaacgatgttgattattacgccgattccgtgaagggcaggttcaccatctccagagacaatgcgaagaacacagtctatttgcagatgaattctctcaagcccgaagacactgctacttattactgtgctctgatgctgaaaggctgggaccattcgacacgttactactggggacagggaacccaggtaacggtgtcatct

SEQ ID NO: 103 - DNA, 357 bp, synthetic construct
  source 1..357: mol_type = other DNA
gaggtacagcttcaggagagcggcggcggatttgtgcaagcaggtgggtccctccgattatcatgcgctgcatccggtaacctgatggtccgtcgggaaatgggctggttccgccaggcacctgggaaagagagggagttcgttgccgccattagcagaagcagtcaggaggaagtttattacgccgactccgtcaagggcaggtttaccatcagtagagacaatgcgaaaaacacagtgtatctgcagatgaattctttgaagcccgaagatactgctacgtattactgtgctatggaagggttctacgtgtacaaccagtattggggacagggaacccaagtgacagtgtcatct

SEQ ID NO: 104 - DNA, 357 bp, synthetic construct
  source 1..357: mol_type = other DNA
gaggtgcaactgcaggagagcggcgggggatttgtgcaggcaggggggtccctccgactttcatgcgctgcatccggtagaagagtaacggtcagtgaaatgggctggttccgccaggcacctggtaaagagagggagtttgttgccgccatcagcatgcgggaacgggaaagcatgtattacgccgactccgtgaagggcaggttcaccattagtcgtgacaatgctaaaaacaccgtctatctgcagatgaactcgttgaagcccgaagatactgctacatattactgtgctgagtacactcactggtactctcatccatattggggacagggaacccaagttacagtgtcatct

SEQ ID NO: 105 - protein, 9 aa, synthetic construct
FTFSSYAMH

SEQ ID NO: 106 - protein, 14 aa, synthetic construct
WVAVISYDGSNKYY

SEQ ID NO: 107 - protein, 16 aa, synthetic construct
RGSGYSYGPGYDAFDI

SEQ ID NO: 108 - protein, 10 aa, synthetic construct
SGSIASNYVQ

SEQ ID NO: 109 - protein, 12 aa, synthetic construct
TTVIYEDNQRPS

SEQ ID NO: 110 - protein, 8 aa, synthetic construct
QSWDPVFG

SEQ ID NO: 111 - protein, 9 aa, synthetic construct
GTFSSYAIS

SEQ ID NO: 112 - protein, 14 aa, synthetic construct
WMGGIIPIFGTANY

SEQ ID NO: 113 - protein, 14 aa, synthetic construct
REGGAVGYYYGMDV

SEQ ID NO: 114 - protein, 11 aa, synthetic construct
SSDVGGYNYVS

SEQ ID NO: 115 - protein, 11 aa, synthetic construct
LMIYEVSNRPS

SEQ ID NO: 116 - protein, 10 aa, synthetic construct
SSYDRSNRSM

SEQ ID NO: 117 - protein, 45 aa, Homo sapiens
TTTPAPRPPTPAPTIASQPLSLRPEACRPAAGGAVHTRGLDFACD

SEQ ID NO: 118 - protein, 12 aa, Homo sapiens
ESKYGPPCPPCP

SEQ ID NO: 119 - protein, 12 aa, Homo sapiens
EPKSPDKTHTCP

SEQ ID NO: 120 - protein, 12 aa, Homo sapiens
EPKSCDKTHTCP

SEQ ID NO: 121 - protein, 236 aa, Homo sapiens
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 122 - protein, 24 aa, Homo sapiens
IYIWAPLAGTCGVLLLSLVITLYC

SEQ ID NO: 123 - protein, 27 aa, Homo sapiens
FWVLVVVGGVLACYSLLVTVAFIIFWV

SEQ ID NO: 124 - protein, 42 aa, Homo sapiens
KRGRKKLLYIFKQPFMRPVQTTQEEDGCSCRFPEEEEGGCEL

SEQ ID NO: 125 - protein, 41 aa, Homo sapiens
RSKRSRGGHSDYMNMTPRRPGPTRKHYQPYAPPRDFAAYRS

SEQ ID NO: 126 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 128 - protein, 112 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQARRA

SEQ ID NO: 129 - DNA, 135 bp, Homo sapiens
  source 1..135: mol_type = genomic DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 130 - DNA, 36 bp, Homo sapiens
  source 1..36: mol_type = genomic DNA
gagtcaaagtatgggcctccatgtcctccatgtccg

SEQ ID NO: 131 - DNA, 36 bp, Homo sapiens
  source 1..36: mol_type = genomic DNA
gagccgaagtcgccagacaaaactcacacttgtcct

SEQ ID NO: 132 - DNA, 708 bp, Homo sapiens
  source 1..708: mol_type = genomic DNA
gcagagccgaagtcgccagacaaaactcacacttgtcctccttgtccagccccccccgtggcgggtcccagcgtgttcctgtttcctccgaagccaaaagataccctgatgatcgcacgcacccccgaagtaacgtgcgtggtggtcgatgtgtcacatgaggaccctgaggtcaaattcaattggtacgttgacggggtagaagttcacaacgctaaaaccaagccaagggaggagcagtacaacagcacctatcgagtggtgagtgtactgaccgtcctacaccaagattggttgaatggcaaggaatacaagtgtaaggtgtccaacaaggctttacctgctcctatcgagaagacaatttctaaggccaaaggccagcccagagagccacaggtttacacactcccaccatcacgtgacgagcttacgaaaaatcaggtcagtctgacttgcctcgttaaaggattttaccctagtgacatagccgtggaatgggaaagcaacggccagcccgagaataattataaaacaacaccgcccgtgctcgactctgatggttcttttttcctgtattccaaactgaccgtcgataagagccggtggcagcagggaaacgtgttctcctgctccgtcatgcatgaagccttgcataaccactatactcaaaagtcactgtctcttagccctgggaagaaagatcccaag

SEQ ID NO: 133 - DNA, 72 bp, Homo sapiens
  source 1..72: mol_type = genomic DNA
atctacatctgggcgcccttggccgggacttgtggggtccttctcctgtcactggttatcaccctttactgc

SEQ ID NO: 134 - DNA, 81 bp, Homo sapiens
  source 1..81: mol_type = genomic DNA
ttctgggtgctggtggtcgtgggcggcgtgctggcctgttacagcctgctcgtgaccgtggccttcatcatcttttgggtc

SEQ ID NO: 135 - DNA, 126 bp, Homo sapiens
  source 1..126: mol_type = genomic DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 136 - DNA, 123 bp, Homo sapiens
  source 1..123: mol_type = genomic DNA
cgaagcaagcggagcagaggcggccacagcgactacatgaacatgacccccagacggcctggccccacccggaagcactaccagccttacgcccctcccagagacttcgccgcctacagaagc

SEQ ID NO: 137 - DNA, 336 bp, Homo sapiens
  source 1..336: mol_type = genomic DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 138 - DNA, 339 bp, Homo sapiens
  source 1..339: mol_type = genomic DNA
agagtgaagttcagccgcagcgccgacgcccctgcctaccagcagggacagaaccagctgtacaacgagctgaacctgggcagacgggaagagtacgacgtgctggacaagcggagaggccgggaccctgagatgggcggcaagccccagcggcggaagaacccccaggaaggcctgtataacgaactgcagaaagacaagatggccgaggcctacagcgagatcggcatgaagggcgagcggcggagaggcaagggccacgatggcctgtaccagggcctgagcaccgccaccaaggacacctacgacgccctgcacatgcaggccctgccccctagg

SEQ ID NO: 139 - DNA, 336 bp, Homo sapiens
  source 1..336: mol_type = genomic DNA
agagtgaagttcagccgcagcgccgacgcccctgcctaccagcagggacagaaccagctgtacaacgagctgaacctgggcagacgggaagagtacgacgtgctggacaagcggagaggccgggaccctgagatgggcggcaagccccagcggcggaagaacccccaggaaggcctgtataacgaactgcagaaagacaagatggccgaggcctacagcgagatcggcatgaagggcgagcggcggagaggcaagggccacgatggcctgtaccagggcctgagcaccgccaccaaggacacctacgacgccctgcacatgcaggcccggcgcgcc